SEQUENCE LISTING (80 sequences)

SEQ ID NO: 1 - DNA, 369 bp, synthetic construct
  misc_feature 1..369: note = Synthetic
  source 1..369: mol_type = other DNA
gaagtgcagctggtggagtctgggggaggcttggttcagcctggcaggtccctaagactctcctgtgcagcctctggattcacctttgatgattatgccttgcactgggtccggcaagctccagggaagggcctggagtgggtctcaggtattagttggactggtggtactatagactatgcggactctgtgaagggccgattcaccatctccagagacaacgccaagaactccctgtatctgcaaatgagcagtctgagaactgaggacacggccatatattactgtacaagagatatccgggggaactggaagtacggaggctggttcgacccctggggccagggaaccctggtcaccgtctcctca

SEQ ID NO: 2 - protein, 123 aa, synthetic construct
  REGION 1..123: note = Synthetic
EVQLVESGGGLVQPGRSLRLSCAASGFTFDDYALHWVRQAPGKGLEWVSGISWTGGTIDYADSVKGRFTISRDNAKNSLYLQMSSLRTEDTAIYYCTRDIRGNWKYGGWFDPWGQGTLVTVSS

SEQ ID NO: 3 - DNA, 24 bp, synthetic construct
  misc_feature 1..24: note = Synthetic
  source 1..24: mol_type = other DNA
ggattcacctttgatgattatgcc

SEQ ID NO: 4 - protein, 8 aa, synthetic construct
  REGION 1..8: note = Synthetic
GFTFDDYA

SEQ ID NO: 5 - DNA, 24 bp, synthetic construct
  misc_feature 1..24: note = Synthetic
  source 1..24: mol_type = other DNA
attagttggactggtggtactata

SEQ ID NO: 6 - protein, 8 aa, synthetic construct
  REGION 1..8: note = Synthetic
ISWTGGTI

SEQ ID NO: 7 - DNA, 48 bp, synthetic construct
  misc_feature 1..48: note = Synthetic
  source 1..48: mol_type = other DNA
acaagagatatccgggggaactggaagtacggaggctggttcgacccc

SEQ ID NO: 8 - protein, 16 aa, synthetic construct
  REGION 1..16: note = Synthetic
TRDIRGNWKYGGWFDP

SEQ ID NO: 9 - DNA, 375 bp, synthetic construct
  misc_feature 1..375: note = Synthetic
  source 1..375: mol_type = other DNA
gaggtgcagctggtggagtctgggggaggcttggtacagcctggagggtccctgagactctcctgtgtagcctctggattcaccttcagtagttatgaaatgaactgggtccgccaggctccagggaaggggctggaatgggtttcatacattagtaatagtggaactaccatatactacgcaggctctgtgaagggccgattcaccatctccagagacaacgccaagaagtcactgtatctgcatatgaatagcctgagagccgaggacacggctgtttattactgtgcgagagaaggtacgattttcggaacggttcactactacttcgctttggacgtctggggccaagggaccacggtcaccgtctcctca

SEQ ID NO: 10 - protein, 125 aa, synthetic construct
  REGION 1..125: note = Synthetic
EVQLVESGGGLVQPGGSLRLSCVASGFTFSSYEMNWVRQAPGKGLEWVSYISNSGTTIYYAGSVKGRFTISRDNAKKSLYLHMNSLRAEDTAVYYCAREGTIFGTVHYYFALDVWGQGTTVTVSS

SEQ ID NO: 11 - DNA, 24 bp, synthetic construct
  misc_feature 1..24: note = Synthetic
  source 1..24: mol_type = other DNA
ggattcaccttcagtagttatgaa

SEQ ID NO: 12 - protein, 8 aa, synthetic construct
  REGION 1..8: note = Synthetic
GFTFSSYE

SEQ ID NO: 13 - DNA, 24 bp, synthetic construct
  misc_feature 1..24: note = Synthetic
  source 1..24: mol_type = other DNA
attagtaatagtggaactaccata

SEQ ID NO: 14 - protein, 8 aa, synthetic construct
  REGION 1..8: note = Synthetic
ISNSGTTI

SEQ ID NO: 15 - DNA, 54 bp, synthetic construct
  misc_feature 1..54: note = Synthetic
  source 1..54: mol_type = other DNA
gcgagagaaggtacgattttcggaacggttcactactacttcgctttggacgtc

SEQ ID NO: 16 - protein, 18 aa, synthetic construct
  REGION 1..18: note = Synthetic
AREGTIFGTVHYYFALDV

SEQ ID NO: 17 - DNA, 324 bp, synthetic construct
  misc_feature 1..324: note = Synthetic
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 18 - protein, 108 aa, synthetic construct
  REGION 1..108: note = Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 19 - DNA, 18 bp, synthetic construct
  misc_feature 1..18: note = Synthetic
  source 1..18: mol_type = other DNA
cagagcattagcagctat

SEQ ID NO: 20 - protein, 6 aa, synthetic construct
  REGION 1..6: note = Synthetic
QSISSY

SEQ ID NO: 21-22 - skipped (2 entries)

SEQ ID NO: 23 - DNA, 30 bp, synthetic construct
  misc_feature 1..30: note = Synthetic
  source 1..30: mol_type = other DNA
caacagagttacagtacccctccgatcacc

SEQ ID NO: 24 - protein, 10 aa, synthetic construct
  REGION 1..10: note = Synthetic
QQSYSTPPIT

SEQ ID NO: 25 - DNA, 1350 bp, synthetic construct
  misc_feature 1..1350: note = Synthetic
  source 1..1350: mol_type = other DNA
gaagtgcagctggtggagtctgggggaggcttggttcagcctggcaggtccctaagactctcctgtgcagcctctggattcacctttgatgattatgccttgcactgggtccggcaagctccagggaagggcctggagtgggtctcaggtattagttggactggtggtactatagactatgcggactctgtgaagggccgattcaccatctccagagacaacgccaagaactccctgtatctgcaaatgagcagtctgagaactgaggacacggccatatattactgtacaagagatatccgggggaactggaagtacggaggctggttcgacccctggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccacctgtggcaggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 26 - protein, 449 aa, synthetic construct
  REGION 1..449: note = Synthetic
EVQLVESGGGLVQPGRSLRLSCAASGFTFDDYALHWVRQAPGKGLEWVSGISWTGGTIDYADSVKGRFTISRDNAKNSLYLQMSSLRTEDTAIYYCTRDIRGNWKYGGWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - DNA, 1356 bp, synthetic construct
  misc_feature 1..1356: note = Synthetic
  source 1..1356: mol_type = other DNA
gaggtgcagctggtggagtctgggggaggcttggtacagcctggagggtccctgagactctcctgtgtagcctctggattcaccttcagtagttatgaaatgaactgggtccgccaggctccagggaaggggctggaatgggtttcatacattagtaatagtggaactaccatatactacgcaggctctgtgaagggccgattcaccatctccagagacaacgccaagaagtcactgtatctgcatatgaatagcctgagagccgaggacacggctgtttattactgtgcgagagaaggtacgattttcggaacggttcactactacttcgctttggacgtctggggccaagggaccacggtcaccgtctcctcagcctctacaaagggaccttctgtgtttcctctggctccttgttctagatctacatctgaatctacagctgctctgggatgtctggtgaaggattattttcctgaacctgtgacagtgtcttggaattctggagctctgacatctggagtgcatacatttcctgctgtgctgcagtcttctggactgtattctctgtcttctgtggtgacagtgccttcttcttctctgggaacaaagacatatacatgtaatgtggatcataagccttctaatacaaaggtggataagagagtggaatctaagtatggacctccttgtcctccttgtcctgctcctcctgtggctggaccttctgtgtttctgtttcctcctaagcctaaggatacactgatgatctctagaacacctgaagtgacatgtgtggtggtggatgtgtctcaggaagatcctgaagtgcagtttaattggtatgtggatggagtggaagtgcataatgctaagacaaagcctagagaagaacagtttaattctacatatagagtggtgtctgtgctgacagtgctgcatcaggattggctgaatggaaaggaatataagtgtaaggtgtctaataagggactgccttcttctatcgaaaagacaatctctaaggctaagggacagcctagagaacctcaggtgtatacactgcctccttctcaggaagaaatgacaaagaatcaggtgtctctgacatgtctggtgaagggattttatccttctgatatcgctgtggaatgggaatctaatggacagcctgaaaataattataagacaacacctcctgtgctggattctgatggatctttttttctgtattctagactgacagtggataagtctagatggcaggaaggaaatgtgttttcttgttctgtgatgcatgaagctctgcataatagatttacacagaagtctctgtctctgtctcctggaaagtag

SEQ ID NO: 28 - protein, 451 aa, synthetic construct
  REGION 1..451: note = Synthetic
EVQLVESGGGLVQPGGSLRLSCVASGFTFSSYEMNWVRQAPGKGLEWVSYISNSGTTIYYAGSVKGRFTISRDNAKKSLYLHMNSLRAEDTAVYYCAREGTIFGTVHYYFALDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 29 - DNA, 648 bp, synthetic construct
  misc_feature 1..648: note = Synthetic
  source 1..648: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 30 - protein, 215 aa, synthetic construct
  REGION 1..215: note = Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - DNA, 342 bp, synthetic construct
  misc_feature 1..342: note = Synthetic
  source 1..342: mol_type = other DNA
caggtgcagctggtggagtctgggggagacgtggtccagcctgggaggtccctgagactctcctgtacagcgtctggattcaccttcagtagctttggcatgcactgggtccgccaggctccaggcaaggggctggagtgggtgacatttttatcgtatgatggaagttataaatactatgcagactccgtgaagggccgattcaccatctccagagacaattccaagaacacgctatatctgcaaatgaacagcctgagagccgaagacacggctatcttttactgtgcgagggactggcttgactactggggccagggaaccctggtcaccgtctcctca

SEQ ID NO: 32 - protein, 114 aa, synthetic construct
  REGION 1..114: note = Synthetic
QVQLVESGGDVVQPGRSLRLSCTASGFTFSSFGMHWVRQAPGKGLEWVTFLSYDGSYKYYADSVKGRFTISRDNSKNTLYLQMNSLRAEDTAIFYCARDWLDYWGQGTLVTVSS

SEQ ID NO: 33 - DNA, 24 bp, synthetic construct
  misc_feature 1..24: note = Synthetic
  source 1..24: mol_type = other DNA
ggattcaccttcagtagctttggc

SEQ ID NO: 34 - protein, 8 aa, synthetic construct
  REGION 1..8: note = Synthetic
GFTFSSFG

SEQ ID NO: 35 - DNA, 24 bp, synthetic construct
  misc_feature 1..24: note = Synthetic
  source 1..24: mol_type = other DNA
ttatcgtatgatggaagttataaa

SEQ ID NO: 36 - protein, 8 aa, synthetic construct
  REGION 1..8: note = Synthetic
LSYDGSYK

SEQ ID NO: 37 - DNA, 21 bp, synthetic construct
  misc_feature 1..21: note = Synthetic
  source 1..21: mol_type = other DNA
gcgagggactggcttgactac

SEQ ID NO: 38 - protein, 7 aa, synthetic construct
  REGION 1..7: note = Synthetic
ARDWLDY

SEQ ID NO: 39 - DNA, 1323 bp, synthetic construct
  misc_feature 1..1323: note = Synthetic
  source 1..1323: mol_type = other DNA
caggtgcagctggtggagtctgggggagacgtggtccagcctgggaggtccctgagactctcctgtacagcgtctggattcaccttcagtagctttggcatgcactgggtccgccaggctccaggcaaggggctggagtgggtgacatttttatcgtatgatggaagttataaatactatgcagactccgtgaagggccgattcaccatctccagagacaattccaagaacacgctatatctgcaaatgaacagcctgagagccgaagacacggctatcttttactgtgcgagggactggcttgactactggggccagggaaccctggtcaccgtctcctcagcctctacaaagggaccttctgtgtttcctctggctccttgttctagatctacatctgaatctacagctgctctgggatgtctggtgaaggattattttcctgaacctgtgacagtgtcttggaattctggagctctgacatctggagtgcatacatttcctgctgtgctgcagtcttctggactgtattctctgtcttctgtggtgacagtgccttcttcttctctgggaacaaagacatatacatgtaatgtggatcataagccttctaatacaaaggtggataagagagtggaatctaagtatggacctccttgtcctccttgtcctgctcctcctgtggctggaccttctgtgtttctgtttcctcctaagcctaaggatacactgatgatctctagaacacctgaagtgacatgtgtggtggtggatgtgtctcaggaagatcctgaagtgcagtttaattggtatgtggatggagtggaagtgcataatgctaagacaaagcctagagaagaacagtttaattctacatatagagtggtgtctgtgctgacagtgctgcatcaggattggctgaatggaaaggaatataagtgtaaggtgtctaataagggactgccttcttctatcgaaaagacaatctctaaggctaagggacagcctagagaacctcaggtgtatacactgcctccttctcaggaagaaatgacaaagaatcaggtgtctctgacatgtctggtgaagggattttatccttctgatatcgctgtggaatgggaatctaatggacagcctgaaaataattataagacaacacctcctgtgctggattctgatggatctttttttctgtattctagactgacagtggataagtctagatggcaggaaggaaatgtgttttcttgttctgtgatgcatgaagctctgcataatagatttacacagaagtctctgtctctgtctcctggaaagtag

SEQ ID NO: 40 - protein, 440 aa, synthetic construct
  REGION 1..440: note = Synthetic
QVQLVESGGDVVQPGRSLRLSCTASGFTFSSFGMHWVRQAPGKGLEWVTFLSYDGSYKYYADSVKGRFTISRDNSKNTLYLQMNSLRAEDTAIFYCARDWLDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 41 - DNA, 387 bp, synthetic construct
  misc_feature 1..387: note = Synthetic
  source 1..387: mol_type = other DNA
gaggtgcagctggtggagtctgggggaggcttggtacagcctggagggtccctgagactctcctgtgaagcctctggattcaccttcagtagttatgaaatgaactgggtccggcaggctccagggaaggggctggagtggatttcacacattagtagtagtggtagttccatatactacgcagactctgtgcagggccgattcaccatctccagagacaaggccaagaactcactgtatctacaaatgaacagcctgagagccgaggacacggctgtttattactgtacgagagaaggatttgagtatggcagttcgtcggggctaaagtactactactacggtttggacgtctggggccaagggaccacggtcaccgtctcctca

SEQ ID NO: 42 - protein, 129 aa, synthetic construct
  REGION 1..129: note = Synthetic
EVQLVESGGGLVQPGGSLRLSCEASGFTFSSYEMNWVRQAPGKGLEWISHISSSGSSIYYADSVQGRFTISRDKAKNSLYLQMNSLRAEDTAVYYCTREGFEYGSSSGLKYYYYGLDVWGQGTTVTVSS

SEQ ID NO: 43 - DNA, 24 bp, synthetic construct
  misc_feature 1..24: note = Synthetic
  source 1..24: mol_type = other DNA
attagtagtagtggtagttccata

SEQ ID NO: 44 - protein, 8 aa, synthetic construct
  REGION 1..8: note = Synthetic
ISSSGSSI

SEQ ID NO: 45 - DNA, 66 bp, synthetic construct
  misc_feature 1..66: note = Synthetic
  source 1..66: mol_type = other DNA
acgagagaaggatttgagtatggcagttcgtcggggctaaagtactactactacggtttggacgtc

SEQ ID NO: 46 - protein, 22 aa, synthetic construct
  REGION 1..22: note = Synthetic
TREGFEYGSSSGLKYYYYGLDV

SEQ ID NO: 47 - DNA, 1368 bp, synthetic construct
  misc_feature 1..1368: note = Synthetic
  source 1..1368: mol_type = other DNA
gaggtgcagctggtggagtctgggggaggcttggtacagcctggagggtccctgagactctcctgtgaagcctctggattcaccttcagtagttatgaaatgaactgggtccggcaggctccagggaaggggctggagtggatttcacacattagtagtagtggtagttccatatactacgcagactctgtgcagggccgattcaccatctccagagacaaggccaagaactcactgtatctacaaatgaacagcctgagagccgaggacacggctgtttattactgtacgagagaaggatttgagtatggcagttcgtcggggctaaagtactactactacggtttggacgtctggggccaagggaccacggtcaccgtctcctcagcctctacaaagggaccttctgtgtttcctctggctccttgttctagatctacatctgaatctacagctgctctgggatgtctggtgaaggattattttcctgaacctgtgacagtgtcttggaattctggagctctgacatctggagtgcatacatttcctgctgtgctgcagtcttctggactgtattctctgtcttctgtggtgacagtgccttcttcttctctgggaacaaagacatatacatgtaatgtggatcataagccttctaatacaaaggtggataagagagtggaatctaagtatggacctccttgtcctccttgtcctgctcctcctgtggctggaccttctgtgtttctgtttcctcctaagcctaaggatacactgatgatctctagaacacctgaagtgacatgtgtggtggtggatgtgtctcaggaagatcctgaagtgcagtttaattggtatgtggatggagtggaagtgcataatgctaagacaaagcctagagaagaacagtttaattctacatatagagtggtgtctgtgctgacagtgctgcatcaggattggctgaatggaaaggaatataagtgtaaggtgtctaataagggactgccttcttctatcgaaaagacaatctctaaggctaagggacagcctagagaacctcaggtgtatacactgcctccttctcaggaagaaatgacaaagaatcaggtgtctctgacatgtctggtgaagggattttatccttctgatatcgctgtggaatgggaatctaatggacagcctgaaaataattataagacaacacctcctgtgctggattctgatggatctttttttctgtattctagactgacagtggataagtctagatggcaggaaggaaatgtgttttcttgttctgtgatgcatgaagctctgcataatagatttacacagaagtctctgtctctgtctcctggaaagtag

SEQ ID NO: 48 - protein, 455 aa, synthetic construct
  REGION 1..455: note = Synthetic
EVQLVESGGGLVQPGGSLRLSCEASGFTFSSYEMNWVRQAPGKGLEWISHISSSGSSIYYADSVQGRFTISRDKAKNSLYLQMNSLRAEDTAVYYCTREGFEYGSSSGLKYYYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 49 - DNA, 360 bp, synthetic construct
  misc_feature 1..360: note = Synthetic
  source 1..360: mol_type = other DNA
caggtgcagctggtacagtctgggactgaggtgaagaagcctggggcctcagtgaaggtctcctgcaaggcttctggatacaccttcaccgcctactatatgcactgggtgcgacaggcccctggtcaaggacttgactggatgggatggatcagccctaacagtggtttcacaaactatgcacagaagtttcagggcagggtcaccatgaccagggacacgtccatcaacacattttatatggagctgagtggactgagatctgacgacacggccgtatattactgtgcgcgagagggttctactcaccacaattctttcgacccctggggccagggaaccctggtcaccgtctcctca

SEQ ID NO: 50 - protein, 120 aa, synthetic construct
  REGION 1..120: note = Synthetic
QVQLVQSGTEVKKPGASVKVSCKASGYTFTAYYMHWVRQAPGQGLDWMGWISPNSGFTNYAQKFQGRVTMTRDTSINTFYMELSGLRSDDTAVYYCAREGSTHHNSFDPWGQGTLVTVSS

SEQ ID NO: 51 - DNA, 24 bp, synthetic construct
  misc_feature 1..24: note = Synthetic
  source 1..24: mol_type = other DNA
ggatacaccttcaccgcctactat

SEQ ID NO: 52 - protein, 8 aa, synthetic construct
  REGION 1..8: note = Synthetic
GYTFTAYY

SEQ ID NO: 53 - DNA, 24 bp, synthetic construct
  misc_feature 1..24: note = Synthetic
  source 1..24: mol_type = other DNA
atcagccctaacagtggtttcaca

SEQ ID NO: 54 - protein, 8 aa, synthetic construct
  REGION 1..8: note = Synthetic
ISPNSGFT

SEQ ID NO: 55 - DNA, 39 bp, synthetic construct
  misc_feature 1..39: note = Synthetic
  source 1..39: mol_type = other DNA
gcgcgagagggttctactcaccacaattctttcgacccc

SEQ ID NO: 56 - protein, 13 aa, synthetic construct
  REGION 1..13: note = Synthetic
AREGSTHHNSFDP

SEQ ID NO: 57 - DNA, 357 bp, synthetic construct
  misc_feature 1..357: note = Synthetic
  source 1..357: mol_type = other DNA
caggttcagctggtgcagtctggagctgaggtgaagaagcctggggcctcagtgaaggtctcctgcaaggcttctgcttacacctttaccagctatggtttcagctgggtgcgacaggcccctggacaagggcttgagtggatgggatggatcagcacttccaatggttacacaaactatgcacagaagatccaggacagagtcaccatgaccacagacacatccacgagcacagcctacatggagctgaggagcctgagatctgacgacacggccgtgtattattgtgcgagcctctctaactgggtatctttctttgactactggggccagggaaccctggtcaccgtctcctca

SEQ ID NO: 58 - protein, 119 aa, synthetic construct
  REGION 1..119: note = Synthetic
QVQLVQSGAEVKKPGASVKVSCKASAYTFTSYGFSWVRQAPGQGLEWMGWISTSNGYTNYAQKIQDRVTMTTDTSTSTAYMELRSLRSDDTAVYYCASLSNWVSFFDYWGQGTLVTVSS

SEQ ID NO: 59 - DNA, 24 bp, synthetic construct
  misc_feature 1..24: note = Synthetic
  source 1..24: mol_type = other DNA
gcttacacctttaccagctatggt

SEQ ID NO: 60 - protein, 8 aa, synthetic construct
  REGION 1..8: note = Synthetic
AYTFTSYG

SEQ ID NO: 61 - DNA, 24 bp, synthetic construct
  misc_feature 1..24: note = Synthetic
  source 1..24: mol_type = other DNA
atcagcacttccaatggttacaca

SEQ ID NO: 62 - protein, 8 aa, synthetic construct
  REGION 1..8: note = Synthetic
ISTSNGYT

SEQ ID NO: 63 - DNA, 36 bp, synthetic construct
  misc_feature 1..36: note = Synthetic
  source 1..36: mol_type = other DNA
gcgagcctctctaactgggtatctttctttgactac

SEQ ID NO: 64 - protein, 12 aa, synthetic construct
  REGION 1..12: note = Synthetic
ASLSNWVSFFDY

SEQ ID NO: 65 - DNA, 324 bp, synthetic construct
  misc_feature 1..324: note = Synthetic
  source 1..324: mol_type = other DNA
gaaatagttttgacacagagtcccggcacactgtcactctctcccggggaaagagccaccttgtcatgtagagcaagtcagtcagtctctagctcttatctcgcctggtaccagcagaagccgggacaggcccctagactgctgatctacggggcaagttccagggccaccggaatccccgaccggttcagtggaagcggaagcggaaccgattttactttgacgatttctagactggagccagaggatttcgccgtttactattgtcaacagtacggaagcagcccgtggacgtttggccagggcacgaaggtagaaatcaag

SEQ ID NO: 66 - protein, 108 aa, synthetic construct
  REGION 1..108: note = Synthetic
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 67 - DNA, 21 bp, synthetic construct
  misc_feature 1..21: note = Synthetic
  source 1..21: mol_type = other DNA
cagtcagtctctagctcttat

SEQ ID NO: 68 - protein, 7 aa, synthetic construct
  REGION 1..7: note = Synthetic
QSVSSSY

SEQ ID NO: 69-70 - skipped (2 entries)

SEQ ID NO: 71 - DNA, 27 bp, synthetic construct
  misc_feature 1..27: note = Synthetic
  source 1..27: mol_type = other DNA
caacagtacggaagcagcccgtggacg

SEQ ID NO: 72 - protein, 9 aa, synthetic construct
  REGION 1..9: note = Synthetic
QQYGSSPWT

SEQ ID NO: 73 - DNA, 1341 bp, synthetic construct
  misc_feature 1..1341: note = Synthetic
  source 1..1341: mol_type = other DNA
caggtgcagctggtacagtctgggactgaggtgaagaagcctggggcctcagtgaaggtctcctgcaaggcttctggatacaccttcaccgcctactatatgcactgggtgcgacaggcccctggtcaaggacttgactggatgggatggatcagccctaacagtggtttcacaaactatgcacagaagtttcagggcagggtcaccatgaccagggacacgtccatcaacacattttatatggagctgagtggactgagatctgacgacacggccgtatattactgtgcgcgagagggttctactcaccacaattctttcgacccctggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccacctgtggcaggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 74 - protein, 446 aa, synthetic construct
  REGION 1..446: note = Synthetic
QVQLVQSGTEVKKPGASVKVSCKASGYTFTAYYMHWVRQAPGQGLDWMGWISPNSGFTNYAQKFQGRVTMTRDTSINTFYMELSGLRSDDTAVYYCAREGSTHHNSFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 75 - DNA, 1338 bp, synthetic construct
  misc_feature 1..1338: note = Synthetic
  source 1..1338: mol_type = other DNA
caggttcagctggtgcagtctggagctgaggtgaagaagcctggggcctcagtgaaggtctcctgcaaggcttctgcttacacctttaccagctatggtttcagctgggtgcgacaggcccctggacaagggcttgagtggatgggatggatcagcacttccaatggttacacaaactatgcacagaagatccaggacagagtcaccatgaccacagacacatccacgagcacagcctacatggagctgaggagcctgagatctgacgacacggccgtgtattattgtgcgagcctctctaactgggtatctttctttgactactggggccagggaaccctggtcaccgtctcctcagcctctacaaagggaccttctgtgtttcctctggctccttgttctagatctacatctgaatctacagctgctctgggatgtctggtgaaggattattttcctgaacctgtgacagtgtcttggaattctggagctctgacatctggagtgcatacatttcctgctgtgctgcagtcttctggactgtattctctgtcttctgtggtgacagtgccttcttcttctctgggaacaaagacatatacatgtaatgtggatcataagccttctaatacaaaggtggataagagagtggaatctaagtatggacctccttgtcctccttgtcctgctcctcctgtggctggaccttctgtgtttctgtttcctcctaagcctaaggatacactgatgatctctagaacacctgaagtgacatgtgtggtggtggatgtgtctcaggaagatcctgaagtgcagtttaattggtatgtggatggagtggaagtgcataatgctaagacaaagcctagagaagaacagtttaattctacatatagagtggtgtctgtgctgacagtgctgcatcaggattggctgaatggaaaggaatataagtgtaaggtgtctaataagggactgccttcttctatcgaaaagacaatctctaaggctaagggacagcctagagaacctcaggtgtatacactgcctccttctcaggaagaaatgacaaagaatcaggtgtctctgacatgtctggtgaagggattttatccttctgatatcgctgtggaatgggaatctaatggacagcctgaaaataattataagacaacacctcctgtgctggattctgatggatctttttttctgtattctagactgacagtggataagtctagatggcaggaaggaaatgtgttttcttgttctgtgatgcatgaagctctgcataatagatttacacagaagtctctgtctctgtctcctggaaagtag

SEQ ID NO: 76 - protein, 445 aa, synthetic construct
  REGION 1..445: note = Synthetic
QVQLVQSGAEVKKPGASVKVSCKASAYTFTSYGFSWVRQAPGQGLEWMGWISTSNGYTNYAQKIQDRVTMTTDTSTSTAYMELRSLRSDDTAVYYCASLSNWVSFFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 77 - DNA, 648 bp, synthetic construct
  misc_feature 1..648: note = Synthetic
  source 1..648: mol_type = other DNA
gaaatagttttgacacagagtcccggcacactgtcactctctcccggggaaagagccaccttgtcatgtagagcaagtcagtcagtctctagctcttatctcgcctggtaccagcagaagccgggacaggcccctagactgctgatctacggggcaagttccagggccaccggaatccccgaccggttcagtggaagcggaagcggaaccgattttactttgacgatttctagactggagccagaggatttcgccgtttactattgtcaacagtacggaagcagcccgtggacgtttggccagggcacgaaggtagaaatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 78 - protein, 215 aa, synthetic construct
  REGION 1..215: note = Synthetic
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 79 - protein, 249 aa, synthetic construct
  REGION 1..249: note = Synthetic
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTEQKLISEEDLGGEQKLISEEDLHHHHHH

SEQ ID NO: 80 - protein, 191 aa, synthetic construct
  REGION 1..191: note = Synthetic
LQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQEQKLISEEDLGGEQKLISEEDLHHHHHH

(19) United States
(12) Patent Application Publication
(10) Pub. No.: US 2025/0043017 A1
(43) Pub. Date: Feb. 6, 2025

(54) BISPECIFIC PD-L1x4-1BB ANTIBODIES AND METHODS OF USE THEREOF

(71) Applicant: Regeneron Pharmaceuticals, Inc., Tarrytown, NY (US)

(72) Inventors: Saida Dadi-Mehmetaj, Ardsley, NY (US); Lauric Haber, Rye Brook, NY (US); Jessica Kirshner, New York, NY (US); Lauren Canova-Kleinman, White Plains, NY (US); Erica Ullman, Yorktown Heights, NY (US)

(21) Appl. No.: 18/766,802

(22) Filed: Jul. 9, 2024

Related U.S. Application Data
(60) Provisional application No. 63/645,718, filed on May 10, 2024.
(60) Provisional application No. 63/512,719, filed on Jul. 10, 2023.

(51) Int. Cl. C07K 16/28; A61K 39/00; A61P 35/00

(52) U.S. Cl. CPC C07K 16/2878; A61P 35/00; C07K 16/2827; A61K 2039/505; C07K 2317/31; C07K 2317/73; C07K 2317/76; C07K 2317/92

(57) ABSTRACT

The present disclosure provides bispecific antigen-binding molecules that bind to PD-L1 and 4-1BB (PD-L1×4-1BB). In certain embodiments, the present disclosure provides a bispecific PD-L1×4-1BB antibody or antigen-binding fragment thereof comprising a first antigen-binding domain that specifically binds 4-1BB, and a second antigen-binding domain that specifically binds PD-L1. In certain embodiments, the bispecific antibodies disclosed herein bind 4-1BB on T-cells with the first antigen-binding domain and PD-L1 expressed on tumor cells or antigen presenting cells with the second antigen-binding domain. In certain embodiments, the antibodies of the present disclosure are useful in treatment of a cancer.

### FIELD

The present disclosure relates to antibodies that bind to PD-L1 and 4-1BB and methods of use thereof, e.g., for treating or preventing cancer.

### SEQUENCE LISTING

The instant application contains a Sequence Listing which has been submitted electronically in XML format and is hereby incorporated by reference in its entirety. Said XML copy, created on Jun. 24, 2024, is named SeqList11375 and is 112,849 bytes in size. The sequence listing is part of the specification and is incorporated in its entirety by reference herein. The following sequences are below the minimum length permitted under ST.26 format for sequence listings: gctgcatcc (SEQ ID NO: 21), AAS (SEQ ID NO: 22), ggggcaagt (SEQ ID NO: 69), and GAS (SEQ ID NO: 70).

### BACKGROUND

The ability of T-cells to recognize and kill their cellular targets—such as virally-infected cells or tumor cells—depends on a coordinated set of interactions. Foremost among these is the recognition and binding of the target cell by the T-cell Receptor (TCR) complex (which includes the associated CD3 γ, δ, ε and ζ chains), and this interaction has been referred to as “signal 1” for T-cell activation. The TCR recognizes a viral or tumor peptide presented on the groove of an MHC protein expressed on the surface of the target cell. Because such binding is generally of low-affinity, successful triggering of “signal 1” requires clustering of many TCR complexes along the interface between the T-cell and its target cell; this interface has been referred to as the “immune synapse”. T-cell activation can be further promoted by additional interactions. For example, T-cells have a molecule referred to as 4-1BB on their surface, which can provide a co-stimulatory “signal 2” to augment the activation via the TCR complex. When a T-cell recognizes its target cell via its TCR complex, and then also engages “signal 2” via 4-1BB binding to its cognate ligand(s) on the target cell, T-cell activation is enhanced; as with “signal 1”, 4-1BB-mediated “signal 2” is thought to occur via co-clustering at the immune synapse.

4-1BB (CD137/ILA/TNFRSF9) has been garnering attention as a promising therapeutic target in the setting of cancer, amongst other diseases, due to its broad expression profile and ability to stimulate various signaling pathways involved in the generation of a potent immune response. As a prominent mediator of immune responses expressed on various cell types, 4-1BB signaling not only exerts protective effects, but is also capable of driving pathologies such as the adverse effects observed following administration of therapeutic 4-1BB antibodies. The persistent stimulation of 4-1BB signaling and, consequently, continuous activation of T cells have been shown to result in granuloma formation in tumor-draining lymph nodes due to the excessive recruitment of macrophages (Kim et al. Cell Mol Immunol (2021) 18 (8): 1956-68). Furthermore, liver-associated toxicity has been reported to be a common problem associated with therapeutic 4-1BB antibody treatment. It has been showed that 4-1BB antibody treatment results in CD8⁺ T cell infiltration into the liver causing inflammation and increased transaminase expression (Dubrot et al. Cancer Immunol Immunother (2010) 59 (8): 1223-33). Such infiltration, however, was not associated with clinical benefit in the setting of tumors in or around the liver tissue. Localized or targeted use of anti-4-1BB mAb can be used for promotion of antitumor immunity with less risk.

Programmed death ligand 1 (PD-L1), a ligand of programmed cell death 1 (PD-1 or PD1), is expressed on antigen-presenting cells such as activated monocytes and dendritic cells, as well as on certain cancer cells. It is known that that stimulation by PD-L1 suppresses activation (cellular proliferation and induction of various cytokine production) of PD-1 expressing T lymphocytes.

Monoclonal antibodies (mAbs) aimed at enhancing T-cell activation are under clinical development as anti-tumor therapeutics. The majority of current treatments, however, have a difficult time overcoming the inhibitory nature of the tumor microenvironment, thus failing to generate efficient tumor-specific T-cell activation and subsequent tumor cell killing. Several blocking mAbs directed against checkpoint inhibitors such as CTLA-4 (cytotoxic T lymphocyte-associated protein) and programmed cell death 1 (PD-1)/programmed cell death ligand 1 (PD-L1) have been clinically approved for melanoma, renal cell carcinoma, non-small lung cancer and advanced metastatic cutaneous squamous cell carcinoma. Blocking PD-1 releases the break on T-cell activation, but its efficacy as a single agent often it is not sufficient to achieve tumor clearance and durable anti-tumor responses. Therefore, there is a need for additional treatments for cancer, such as improved immunotherapeutic agents.

### SUMMARY

The present disclosure provides bispecific antigen-binding molecules including bi-specific antibodies comprising a first antigen-binding arm (or domain) that specifically binds 4-1BB (also known as CD137 and tumor necrosis factor receptor superfamily 9 (TNFRSF9)) and a second antigen-binding arm (or domain) that binds programmed death ligand 1 (PD-L1) (“PD-L1×4-1BB” or “4-1BB×PD-L1” antibodies) that induce long lived anti-tumor immunity and promote robust intra-tumoral T-cell activation with decreased systemic cytokine secretion. The bispecific antigen-binding molecules of the present disclosure provide a pan-tumor costimulatory approach (tumor-agnostic targeting) whereby inhibitory checkpoint PD-L1 which is present across various tumor types is combined with a costimulatory signal activating 4-1BB on T cells. The PD-L1×4-1BB bispecific antibodies block PD-L1 interaction with PD-1 and exploit PD-L1 expression in tumor or tumor microenvironment to anchor the 4-1BB agonist antigen-binding fragment intratumorally, which provides co-stimulatory signal to T cells in a more spatially restricted manner. The PD-L1×4-1BB bispecific antigen-binding molecules of the present disclosure bind to and engage PD-L1 expressing tumor cells, APCs and other tumor-infiltrating immune cells, thus providing a broad tumor-agnostic targeting as compared to a bispecific targeting 4-1BB and a tumor associated antigen (TAA). In certain embodiments, the bispecific antigen-binding molecules enhance the T cell mediated killing of tumor cells expressing PD-L1 and a TAA, when used in combination with a CD3-based bispecific targeted to that TAA. Collectively, combining 4-1BB-agonism with PD-1 inhibition may provide safe, biologic solutions with strikingly enhanced, specific and synergistic anti-tumor activity across various tumor types.

In one aspect, the present disclosure provides an isolated bispecific antigen-binding molecule comprising a first antigen-binding domain that binds human 4-1BB with a K_{D} of less than about 2×10⁻⁷ M as measured by surface plasmon resonance at 25° C.; and a second antigen-binding domain that specifically binds a programmed death-ligand 1 (PD-L1) with a K_{D} of less than about 2×10⁻¹⁰M as measured by surface plasmon resonance at 25° C.

In some embodiments, the bispecific antigen-binding molecule binds to the surface of human T cells with an EC₅₀ of less than about 4×10⁻⁹ M as measured by an in vitro FACS binding assay.

In some embodiments, the bispecific antigen-binding molecule binds to the surface of a cell expressing PD-L1 with an EC₅₀ of less than about 4×10⁻⁹ M as measured by an in vitro FACS binding assay.

In some embodiments, the bispecific antigen-binding molecule blocks PD-L1 binding to PD-1 with an IC50 of less than about 1.3 nM as measured by an ELISA-based blocking assay.

In another aspect, the isolated bispecific antigen-binding molecule demonstrates a costimulatory effect when used in conjunction with an anti-Mucin 16 (MUC16)×CD3 bispecific antibody and tested on target cells expressing PD-L1.

In some embodiments, the bispecific antigen-binding molecule, in combination with a bispecific MUC16×CD3 antibody, mediates in vitro T cell killing of OVCAR-3 cells expressing PD-L1 with an EC₅₀ of less than about 10⁻¹⁰ M.

In certain embodiments of the disclosure, the bi-specific antigen-binding molecule (e.g., antibody or antigen-binding fragment thereof) that binds PD-L1 and 4-1BB includes: (1) a PD-L1 binding arm comprising: (a) a heavy chain immunoglobulin or variable region thereof comprising an amino acid sequence having at least 90%, 91%, 92%, 93%, 94%, 95%, 96%, 97%, 98%, 99% amino acid sequence identity to, comprises, or consists of the amino acid sequence set forth in SEQ ID NOs: 2 and 50, or a variant thereof; and/or (b) a light chain immunoglobulin or variable region thereof comprising an amino acid sequence having at least 90%, 91%, 92%, 93%, 94%, 95%, 96%, 97%, 98%, 99% amino acid sequence identity to, comprises, or consists of the amino acid sequence set forth in SEQ ID NOs: 18 and 66, or a variant thereof; or (2) a 4-1BB binding arm comprising: (c) a heavy chain immunoglobulin or variable region thereof comprising an amino acid sequence having at least 90%, 91%, 92%, 93%, 94%, 95%, 96%, 97%, 98%, 99% amino acid sequence identity to, comprises, or consists of the amino acid sequence set forth in SEQ ID NOs: 10, 32, 42 and 58, or a variant thereof; and/or (d) a light chain immunoglobulin or variable region thereof comprising an amino acid sequence having at least 90%, 91%, 92%, 93%, 94%, 95%, 96%, 97%, 98%, 99% amino acid sequence identity to, comprises, or consists of the amino acid sequence set forth in SEQ ID NOs: 18 and 66.

In an embodiment of the disclosure, the bispecific antigen-binding protein (e.g., antibody or antigen-binding fragment thereof) that binds PD-L1 and 4-1BB includes: (1) a PD-L1 binding arm comprising: (a) a heavy chain immunoglobulin or variable region thereof comprising the HCDR1, HCDR2 and HCDR3 of a heavy chain variable region comprising an amino acid sequence set forth in SEQ ID NO: 2 or 50, and at least 90%, 91%, 92%, 93%, 94%, 95%, 96%, 97%, 98%, 99% amino acid sequence identity to, comprises, or consists of, the amino acid sequence set forth in SEQ ID NO: 2 or 50, respectively; and/or (b) a light chain immunoglobulin or variable region thereof comprising the LCDR1, LCDR2 and LCDR3 of a light chain variable region comprising an amino acid sequence set forth in SEQ ID NO: 18 or 66, and at least 90%, 91%, 92%, 93%, 94%, 95%, 96%, 97%, 98%, 99% amino acid sequence identity to, comprises, or consists of the amino acid sequence set forth in SEQ ID NO: 18 or 66, respectively; and a 4-1BB binding arm; or (2) a 4-1BB binding arm comprising: (c) a heavy chain immunoglobulin or variable region thereof comprising the HCDR1, HCDR2 and HCDR3 of a heavy chain variable region comprising an amino acid sequence set forth in SEQ ID NO: 10, 32, 42 or 58, and at least 90%, 91%, 92%, 93%, 94%, 95%, 96%, 97%, 98%, 99% amino acid sequence identity to, comprises, or consists of the amino acid sequence set forth in SEQ ID NO: 10, 32, 42 or 58, respectively; and/or (d) a light chain immunoglobulin or variable region thereof comprising the LCDR1, LCDR2 and LCDR3 of a light chain variable region comprising an amino acid sequence set forth in SEQ ID NO: 18 or 66, and at least 90%, 91%, 92%, 93%, 94%, 95%, 96%, 97%, 98%, 99% amino acid sequence identity to, comprises, or consists of the amino acid sequence set forth in SEQ ID NO: 18 or 66, respectively; and a PD-L1 binding arm.

In some embodiments, the first antigen-binding domain comprises: (a) three heavy chain complementarity determining regions (HCDR1, HCDR2 and HCDR3) contained within a heavy chain variable region (HCVR) comprising the amino acid sequence selected from the group consisting of SEQ ID NOs: 58, 42, 32 and 10, or a variant thereof; and (b) three light chain complementarity determining regions (LCDR1, LCDR2 and LCDR3) contained within a light chain variable region (LCVR) comprising the amino acid sequence selected from the group consisting of SEQ ID NOs: 66 and 18, or a variant thereof.

In some embodiments, the isolated bispecific antigen-binding molecule comprises a HCDR1 comprising the amino acid sequence selected from the group consisting of SEQ ID NOs: 60, 34 and 12, a HCDR2 comprising the amino acid sequence selected from the group consisting of SEQ ID NOs: 62, 44, 36 and 14, and a HCDR3 comprising the amino acid sequence selected from the group consisting of SEQ ID NOs: 64, 46, 38 and 16.

In some embodiments, the isolated bispecific antigen-binding molecule comprises a LCDR1 comprising the amino acid sequence selected from the group consisting of SEQ ID NOs: 68 and 20, a LCDR2 comprising the amino acid sequence selected from the group consisting of SEQ ID NOs: 70 and 22, and a LCDR3 comprising the amino acid sequence selected from the group consisting of SEQ ID NO: 72 and 24.

In some embodiments, the first antigen-binding domain comprises a HCVR comprising the amino acid sequence of SEQ ID NO: 58, or a variant thereof, and a LCVR comprising the amino acid sequence of SEQ ID NO: 66, or a variant thereof.

In some embodiments, the first antigen-binding domain comprises a HCVR comprising the amino acid sequence of SEQ ID NO: 42, or a variant thereof, and a LCVR comprising the amino acid sequence of SEQ ID NO: 18, or a variant thereof.

In some embodiments, the first antigen-binding domain comprises a HCVR comprising the amino acid sequence of SEQ ID NO: 32, or a variant thereof, and a LCVR comprising the amino acid sequence of SEQ ID NO: 18, or a variant thereof.

In some embodiments, the first antigen-binding domain comprises a HCVR comprising the amino acid sequence of SEQ ID NO: 10, or a variant thereof, and a LCVR comprising the amino acid sequence of SEQ ID NO: 18, or a variant thereof.

In some embodiments, the second antigen-binding domain comprises: (a) three heavy chain complementarity determining regions (HCDR1, HCDR2 and HCDR3) contained within a heavy chain variable region (HCVR) comprising the amino acid sequence selected from the group consisting of SEQ ID NOs: 50 and 2, or a variant thereof; and (b) three light chain complementarity determining regions (LCDR1, LCDR2 and LCDR3) contained within a light chain variable region (LCVR) comprising the amino acid sequence selected from the group consisting of SEQ ID NOs: 66 and 18, or a variant thereof.

In some embodiments, the second antigen-binding domain comprises: (a) a HCDR1 comprising the amino acid sequence of SEQ ID NO: 52 or SEQ ID NO: 4; (b) a HCDR2 comprising the amino acid sequence of SEQ ID NO: 54 or SEQ ID NO: 6; and (c) a HCDR3 comprising the amino acid sequence of SEQ ID NO: 58 or SEQ ID NO: 8.

In some embodiments, the second antigen-binding domain comprises a LCDR1 comprising the amino acid sequence selected from the group consisting of SEQ ID NOs: 68 and 20, a LCDR2 comprising the amino acid sequence selected from the group consisting of SEQ ID NOs: 70 and 22, and a LCDR3 comprising the amino acid sequence selected from the group consisting of SEQ ID NO: 72 and 24.

In some embodiments, the second antigen-binding domain comprises: (a) HCDR1, HCDR2, HCDR3 domains, respectively, comprising the amino acid sequences of SEQ ID NOs: 52, 54, 56; and LCDR1, LCDR2, LCDR3 domains, respectively, comprising the amino acid sequences of SEQ ID NOs: 68, 70, 72; or (b) HCDR1, HCDR2, HCDR3 domains, respectively, comprising the amino acid sequences of SEQ ID NOs: 4, 6, 8; and LCDR1, LCDR2, LCDR3 domains, respectively, comprising the amino acid sequences of SEQ ID NOs: 20, 22, 24.

In some embodiments, the second antigen-binding domain comprises: (a) a HCVR comprising the amino acid sequence of SEQ ID NO: 50, or a variant thereof, and a LCVR comprising the amino acid sequence of SEQ ID NO: 66, or a variant thereof; or (b) a HCVR comprising the amino acid sequence of SEQ ID NO: 2, or a variant thereof, and a LCVR comprising the amino acid sequence of SEQ ID NO: 18, or a variant thereof.

In some aspects, the present disclosure provides an isolated bispecific antigen-binding molecule, comprising: (a) a first antigen-binding domain that specifically binds human 4-1BB wherein the first antigen-binding domain comprises HCDR1, HCDR2, HCDR3 domains, respectively, comprising the amino acid sequences of SEQ ID NOs: 60, 62, 64, and LCDR1, LCDR2, LCDR3 domains, respectively, comprising the amino acid sequences of SEQ ID NOs: 68, 70, 72; and (b) a second antigen-binding domain that specifically binds human PD-L1 wherein the second antigen-binding domain comprises HCDR1, HCDR2, HCDR3 domains, respectively, comprising the amino acid sequences of SEQ ID NOs: 52, 54, 56, and LCDR1, LCDR2, LCDR3 domains, respectively, comprising the amino acid sequences of SEQ ID NOs: 68, 70, 72.

In certain aspects, the present disclosure provides an isolated bispecific antigen-binding molecule, comprising: (a) a first antigen-binding domain that specifically binds human 4-1BB wherein the first antigen-binding domain comprises HCDR1, HCDR2, HCDR3 domains, respectively, comprising the amino acid sequences of SEQ ID NOs: 12, 44, 46, and LCDR1, LCDR2, LCDR3 domains, respectively, comprising the amino acid sequences of SEQ ID NOs: 20, 22, 24; and (b) a second antigen-binding domain that specifically binds human PD-L1 wherein the second antigen-binding domain comprises HCDR1, HCDR2, HCDR3 domains, respectively, comprising the amino acid sequences of SEQ ID NOs: 4, 6, 8, and LCDR1, LCDR2, LCDR3 domains, respectively, comprising the amino acid sequences of SEQ ID NOs: 20, 22, 24.

In other aspects, the present disclosure provides an isolated bispecific antigen-binding molecule, comprising: (a) a first antigen-binding domain that specifically binds human 4-1BB wherein the first antigen-binding domain comprises HCDR1, HCDR2, HCDR3 domains, respectively, comprising the amino acid sequences of SEQ ID NOs: 34, 36, 38, and LCDR1, LCDR2, LCDR3 domains, respectively, comprising the amino acid sequences of SEQ ID NOs: 20, 22, 24; and (b) a second antigen-binding domain that specifically binds human PD-L1 wherein the second antigen-binding domain comprises HCDR1, HCDR2, HCDR3 domains, respectively, comprising the amino acid sequences of SEQ ID NOs: 4, 6, 8, and LCDR1, LCDR2, LCDR3 domains, respectively, comprising the amino acid sequences of SEQ ID NOs: 20, 22, 24.

In some aspects, the present disclosure provides an isolated bispecific antigen-binding molecule, comprising: (a) a first antigen-binding domain that specifically binds human 4-1BB wherein the first antigen-binding domain comprises HCDR1, HCDR2, HCDR3 domains, respectively, comprising the amino acid sequences of SEQ ID NOs: 12, 14, 16, and LCDR1, LCDR2, LCDR3 domains, respectively, comprising the amino acid sequences of SEQ ID NOs: 20, 22, 24; and (b) a second antigen-binding domain that specifically binds human PD-L1 wherein the second antigen-binding domain comprises HCDR1, HCDR2, HCDR3 domains, respectively, comprising the amino acid sequences of SEQ ID NOs: 4, 6, 8, and LCDR1, LCDR2, LCDR3 domains, respectively, comprising the amino acid sequences of SEQ ID NOs: 20, 22, 24.

In some embodiments, the isolated bispecific antigen-binding molecule comprises: (a) a first antigen-binding domain that comprises a HCVR comprising the amino acid sequence of SEQ ID NO: 58, and a LCVR comprising the amino acid sequence of SEQ ID NO: 66; and (b) a second antigen-binding domain that comprises a HCVR comprising the amino acid sequence of SEQ ID NO: 50, and a LCVR comprising the amino acid sequence of SEQ ID NO: 66.

In some embodiments, the isolated bispecific antigen-binding molecule comprises: (a) a first antigen-binding domain that comprises a HCVR comprising the amino acid sequence of SEQ ID NO: 42, and a LCVR comprising the amino acid sequence of SEQ ID NO: 18; and (b) a second antigen-binding domain that comprises a HCVR comprising the amino acid sequence of SEQ ID NO: 2, and a LCVR comprising the amino acid sequence of SEQ ID NO: 18.

In some embodiments, the isolated bispecific antigen-binding molecule comprises: (a) a first antigen-binding domain that comprises a HCVR comprising the amino acid sequence of SEQ ID NO: 32, and a LCVR comprising the amino acid sequence of SEQ ID NO: 18; and (b) a second antigen-binding domain that comprises a HCVR comprising the amino acid sequence of SEQ ID NO: 2, and a LCVR comprising the amino acid sequence of SEQ ID NO: 18.

In some embodiments, the isolated bispecific antigen-binding molecule comprises: (a) a first antigen-binding domain that comprises a HCVR comprising the amino acid sequence of SEQ ID NO: 10, and a LCVR comprising the amino acid sequence of SEQ ID NO: 18; and (b) a second antigen-binding domain that comprises a HCVR comprising the amino acid sequence of SEQ ID NO: 2, and a LCVR comprising the amino acid sequence of SEQ ID NO: 18.

In certain aspects, the present disclosure provides an isolated bispecific antigen-binding molecule that competes for binding to PD-L1 or binds to the same epitope on PD-L1 as a reference antibody, wherein the reference antibody comprises a first antigen-binding domain comprising an HCVR/LCVR pair comprising the amino acid sequences selected from the group consisting of SEQ ID NOs: 58/66, 42/18, 32/18 and 10/18 and a second antigen-binding domain comprising an HCVR/LCVR pair comprising the amino acid sequences selected from the group consisting of SEQ ID NOs: 50/66 and 2/18.

In some aspects, the present disclosure provides an isolated bispecific antigen-binding molecule that competes for binding to human 4-1BB or binds to the same epitope on human 4-1BB as a reference antibody, wherein the reference antibody comprises a first antigen-binding domain comprising an HCVR/LCVR pair comprising the amino acid sequences selected from the group consisting of SEQ ID NOs: 58/66, 42/18, 32/18 and 10/18 and a second antigen-binding domain comprising an HCVR/LCVR pair comprising the amino acid sequences selected from the group consisting of SEQ ID NOs: 50/66 and 2/18.

In some embodiments, the isolated bispecific antigen-binding molecule is a human bispecific antigen-binding molecule.

In some embodiments, the isolated bispecific antigen-binding molecule is a bispecific antibody.

In some embodiments, the antibody comprises a human IgG heavy chain constant region attached, respectively, to the HCVR of each of the first antigen-binding domain and the second antigen-binding domain.

In some embodiments, the heavy chain constant region is of isotype IgG1. In other embodiments, the heavy chain constant region is of isotype IgG4.

In some embodiments, the heavy chain constant region attached to the HCVR of the first antigen-binding domain, or the heavy chain constant region attached to the HCVR of the second antigen-binding domain, but not both, contains an amino acid modification that reduces Protein A binding relative to a heavy chain of the same isotype without the modification.

In some embodiments, the modification comprises a H435R substitution (EU numbering) in a heavy chain of isotype IgG1 or IgG4. In some embodiments, the modification comprises a H435R substitution and a Y436F substitution (EU numbering) in a heavy chain of isotype IgG1 or IgG4.

In some embodiments, the bispecific antibody comprises a chimeric hinge that reduces Fcγ receptor binding relative to a wild-type hinge of the same isotype.

In some embodiments, the antibody comprises a first heavy chain containing the HCVR of the first antigen-binding domain and a second heavy chain containing the HCVR of the second antigen-binding domain, wherein the first heavy chain comprises the amino acid sequence selected from the group consisting of SEQ ID NOs: 76, 48, 40 and 28; and the second heavy chain comprises the amino acid sequence selected from the group consisting of SEQ ID NOs: 74 and 26.

In some embodiments, the antibody comprises a common light chain containing the LCVR of the first and second antigen-binding domains, wherein the common light chain comprises the amino acid sequence selected from the group consisting of SEQ ID NOs: 78 and 30.

In some embodiments, the antibody comprises a first heavy chain containing the HCVR of the first antigen-binding domain and a second heavy chain containing the HCVR of the second antigen-binding domain, wherein the first heavy chain comprises the amino acid sequence of SEQ ID NO: 76 and the second heavy chain comprises the amino acid sequence of SEQ ID NO: 74.

In some embodiments, the antibody comprises a common light chain containing the LCVR of the first and second antigen-binding domains, wherein the common light chain comprises the amino acid sequence of SEQ ID NOs: 78.

In certain aspects, the present disclosure provides a bispecific antibody comprising a first antigen-binding domain that binds specifically to human 4-1BB and a second antigen-binding domain that binds specifically to human PD-L1, wherein the bispecific antibody comprises a first heavy chain comprising the amino acid sequence of SEQ ID NO: 76 paired with a common light chain comprising the amino acid sequence of SEQ ID NO: 78, and a second heavy chain comprising the amino acid sequence of SEQ ID NO: 74 paired with a common light chain comprising the amino acid sequence of SEQ ID NO: 78.

In some aspects, the present disclosure provides a bispecific antibody comprising a first antigen-binding domain that binds specifically to human 4-1BB and a second antigen-binding domain that binds specifically to human PD-L1, wherein the bispecific antibody comprises a first heavy chain comprising the amino acid sequence of SEQ ID NO: 48 paired with a common light chain comprising the amino acid sequence of SEQ ID NO: 30, and a second heavy chain comprising the amino acid sequence of SEQ ID NO: 26 paired with a common light chain comprising the amino acid sequence of SEQ ID NO: 30.

In other aspects, the present disclosure provides a bispecific antibody comprising a first antigen-binding domain that binds specifically to human 4-1BB and a second antigen-binding domain that binds specifically to human PD-L1, wherein the bispecific antibody comprises a first heavy chain comprising the amino acid sequence of SEQ ID NO: 40 paired with a common light chain comprising the amino acid sequence of SEQ ID NO: 30, and a second heavy chain comprising the amino acid sequence of SEQ ID NO: 26 paired with a common light chain comprising the amino acid sequence of SEQ ID NO: 30.

In some other aspects, the present disclosure provides a bispecific antibody comprising a first antigen-binding domain that binds specifically to human 4-1BB and a second antigen-binding domain that binds specifically to human PD-L1, wherein the bispecific antibody comprises a first heavy chain comprising the amino acid sequence of SEQ ID NO: 28 paired with a common light chain comprising the amino acid sequence of SEQ ID NO: 30, and a second heavy chain comprising the amino acid sequence of SEQ ID NO: 26 paired with a common light chain comprising the amino acid sequence of SEQ ID NO: 30.

In some embodiments, the bispecific antibody is a human antibody.

In some aspects, the present disclosure provides a pharmaceutical composition comprising the bispecific antigen-binding molecule as set forth herein, and a pharmaceutically acceptable carrier or diluent.

In other some aspects, the present disclosure provides a pharmaceutical composition comprising the bispecific antibody as set forth herein, and a pharmaceutically acceptable carrier or diluent.

The present disclosure also provides a method for making a bi-specific antigen-binding molecule set forth herein comprising: (a) introducing one or more nucleic acid molecules comprising nucleic acid sequences encoding the immunoglobulin chains of said antigen-binding molecule into a host cell (e.g., a CHO cell); (b) culturing the host cell under conditions favorable to expression of the nucleic acid molecules; and (c) optionally, isolating the antigen-binding molecule or immunoglobulin chain from the host cell and/or medium in which the host cell is grown.

In some embodiments, the host cell is a Chinese hamster ovary (CHO) cell.

In some embodiments, the method further comprises formulating the antigen-binding molecule as a pharmaceutical composition comprising an acceptable carrier.

Any antigen-binding molecule or immunoglobulin chain which is a product of such a method is part of the present disclosure.

The present disclosure also provides a nucleic acid molecule comprising a nucleotide sequence a bispecific antigen-binding molecule set forth herein, or a set of nucleic acid molecules comprising nucleotide sequences encoding the HCVR of the first antigen-binding domain that specifically binds to human 4-1BB, the HCVR of the second antigen-binding domain that specifically binds to human PD-L1, and the LCVR of an isolated bispecific antigen-binding molecule set forth herein. Expression vectors comprising the nucleic acid molecule, or a set of expression vectors comprising the set of nucleic acid molecules of the present disclosure are also part of the present disclosure, as well as host cells (e.g., CHO) comprising a nucleic acid molecule, vector or antigen-binding protein of the present disclosure.

The present disclosure provides a method of producing a bispecific antigen-binding molecule that binds to PD-L1 and 4-1BB comprising: (a) culturing the host cell set forth herein under conditions favorable for production of the bispecific antigen-binding molecule; and (b) optionally, isolating the antigen-binding molecule or immunoglobulin chain from the host cell and/or medium in which the host cell is grown. In some embodiments, the host cell is a CHO cell. In some embodiments, the method further comprises formulating the antigen-binding molecule as a pharmaceutical composition comprising an acceptable carrier.

Antigen-binding molecule or immunoglobulin chain which is a product of the method disclosure herein is also part of the present disclosure.

The present disclosure also provides a nucleic acid molecule comprising a nucleotide sequence encoding a bispecific antibody as set forth herein, or a set of nucleic acid molecules comprising nucleotide sequences encoding the heavy chain of the first antigen-binding domain that specifically binds to human 4-1BB, the heavy of the second antigen-binding domain that specifically binds to human PD-L1, and the light chain of a bispecific antibody as set forth herein. Expression vectors comprising the nucleic acid of the present disclosure are also part of the present disclosure, as well as host cells (e.g., CHO cell) comprising a nucleic acid molecule, vector or antibody of the present disclosure.

The present disclosure provides a method of producing a bispecific antibody that binds to PD-L1 and 4-1BB comprising: (a) culturing the host cell set forth herein under conditions favorable for production of the bispecific antigen-binding molecule; and (b) optionally, isolating the antigen-binding molecule or immunoglobulin chain from the host cell and/or medium in which the host cell is grown. In some embodiments, the host cell is a CHO cell. In some embodiments, the method further comprises formulating the bispecific antibody as a pharmaceutical composition comprising an acceptable carrier.

An antibody which is a product of the method disclosure herein is also part of the present disclosure.

The present disclosure also provides a method for treating a hyperproliferative disease (e.g., cancer), in a subject (e.g., a human) in need thereof, comprising administering (e.g., subcutaneously, intravenously or intramuscularly) an effective amount of a bispecific antigen-binding protein or composition or formulation. In an embodiment of the disclosure, the cancer is a B cell cancer, basal cell carcinoma, bladder urothelial carcinoma, brain cancer, breast cancer, cervical cancer, cervical squamous cell carcinoma, colorectal cancer, diffuse large B cell lymphoma, endometrial adenocarcinoma, endometrial cancer, esophageal carcinoma, gastroesophageal adenocarcinoma, gastroesophageal cancer, glioblastoma multiforme, head & neck squamous cell carcinoma, hepatocellular carcinoma, lung adenocarcinoma, lung cancer, lung squamous cell carcinoma, melanoma, multiple myeloma, non-small cell lung cancer, ovarian cancer, pancreatic cancer, prostate cancer, rectal cancer, skin cancer, or a T cell cancer. In one embodiment, the cancer comprises PD-L1-expressing tumor cells.

In some aspects, the present disclosure provides a method of inhibiting growth of a tumor in a subject, comprising administering an isolated bispecific antigen-binding molecule, or a bispecific antibody, or a pharmaceutical composition as set forth herein to the subject.

In some embodiments, the tumor is a B cell cancer, basal cell carcinoma, bladder urothelial carcinoma, brain cancer, breast cancer, cervical cancer, cervical squamous cell carcinoma, colorectal cancer, diffuse large B cell lymphoma, endometrial adenocarcinoma, endometrial cancer, esophageal carcinoma, gastroesophageal adenocarcinoma, gastroesophageal cancer, glioblastoma multiforme, head & neck squamous cell carcinoma, hepatocellular carcinoma, lung adenocarcinoma, lung cancer, lung squamous cell carcinoma, melanoma, multiple myeloma, non-small cell lung cancer, ovarian cancer, pancreatic cancer, prostate cancer, rectal cancer, skin cancer, or a T cell cancer.

In some embodiments, the tumor expresses PD-L1.

In some embodiments, the method further comprises administering a second therapeutic agent or therapeutic regimen.

In some embodiments, the second therapeutic agent or therapeutic regimen comprises a chemotherapeutic drug, a DNA alkylator, an immunomodulator, a proteasome inhibitor, a histone deacetylase inhibitor, radiotherapy, surgery, a stem cell transplant, a bispecific antibody that interacts with a tumor associated antigen (TAA) and a T cell or immune cell antigen, an antibody drug conjugate, an oncolytic virus, a bispecific antibody conjugated to an anti-tumor agent, a VEGF inhibitor, a checkpoint inhibitor, a GITR agonist, a CD27 agonist, a 4-1BB activator, a PD-1 inhibitor, a CTLA-4 inhibitor, an EGFR inhibitor, Ang2 inhibitor, a MUC16 inhibitor, a cancer vaccine, a cytokine, a modified IL2, a modified IL12, IL4 inhibitor, IL6 inhibitor, a corticosteroid, or combinations thereof.

In some embodiments, the T cell or immune cell antigen is CD3.

In some embodiments, the TAA is selected from the group consisting of AFP, ALK, BAGE proteins, BCMA, BIRC5 (survivin), BIRC7, β-catenin, brc-abl, BRCA1, BORIS, CA9, carbonic anhydrase IX, caspase-8, CALR, CCR5, CD19, CD20 (MS4A1), CD22, CD30, CD40, CDK4, CEA, CTLA4, cyclin-B1, CYP1B1, EGFR, EGFRvIII, ErbB2/Her2, ErbB3, ErbB4, ETV6-AML, EpCAM, EphA2, Fra-1, FOLR1, GAGE proteins (e.g., GAGE-1, -2), GD2, GD3, GloboH, glypican-3, GM3, gp100, Her2, HLA/B-raf, HLA/k-ras, HLA/MAGE-A3, hTERT, LMP2, MAGE proteins (e.g., MAGE-1, -2, -3, -4, -6, and -12), MART-1, mesothelin, ML-IAP, Muc1, Muc2, Muc3, Muc4, Muc5, Muc16 (CA-125), MUM1, NA17, NY-BR1, NY-BR62, NY-BR85, NY-ESO1, OX40, p15, p53, PAP, PAX3, PAX5, PCTA-1, PLAC1, PRLR, PRAME, PSMA (FOLH1), RAGE proteins, Ras, RGS5, Rho, SART-1, SART-3, STEAP1, STEAP2, TAG-72, TGF-β, TMPRSS2, Thompson-nouvelle antigen (Tn), TRP-1, TRP-2, tyrosinase, and uroplakin-3.

In other aspects, the present disclosure provides use of the bispecific antigen-binding molecule, or a bispecific antibody, or a pharmaceutical composition as set forth herein in the treatment of a tumor.

In some embodiments, the tumor is a B cell cancer, basal cell carcinoma, bladder urothelial carcinoma, brain cancer, breast cancer, cervical cancer, cervical squamous cell carcinoma, colon cancer, colorectal cancer, diffuse large B cell lymphoma, endometrial adenocarcinoma, endometrial cancer, esophageal carcinoma, gastroesophageal adenocarcinoma, gastroesophageal cancer, glioblastoma multiforme, head & neck squamous cell carcinoma, hepatocellular carcinoma, melanoma, multiple myeloma, leukemia, lung adenocarcinoma, lung cancer, lung squamous cell carcinoma, lung adenocarcinoma, cervical squamous cell carcinoma, endometrial adenocarcinoma, bladder urothelial carcinoma, lung cancer, non-small cell lung cancer, colorectal cancer, ovarian cancer, pancreatic cancer, prostate cancer, rectal cancer, endometrial cancer, skin cancer, or a T cell cancer.

In some embodiments, the tumor expresses PD-L1.

In some embodiments, the antigen-binding molecule or pharmaceutical composition is for use in combination with a second therapeutic agent or therapeutic regimen that comprises a chemotherapeutic drug, a DNA alkylator, an immunomodulator, a proteasome inhibitor, a histone deacetylase inhibitor, radiotherapy, surgery, a stem cell transplant, a bispecific antibody that interacts with a tumor associated antigen (TAA) and a T cell or immune cell antigen, an antibody drug conjugate, an oncolytic virus, a bispecific antibody conjugated to an anti-tumor agent, a VEGF inhibitor, a checkpoint inhibitor, a GITR agonist, a CD27 agonist, a 4-1BB activator, a PD-1 inhibitor, a CTLA-4 inhibitor, an EGFR inhibitor, Ang2 inhibitor, a MUC16 inhibitor, a cancer vaccine, a cytokine, a modified IL2, a modified IL12, IL4 inhibitor, IL6 inhibitor, a corticosteroid, or combinations thereof.

### BRIEF DESCRIPTION OF THE FIGURES

FIG. 1 relates to Example 7 and is a graph showing average tumor volume in mice administered the indicated antibodies.

FIGS. 2A-2B relate to Example 8. FIG. 2A is a graph showing average tumor volume in mice administered the indicated antibodies at the indicated doses. FIG. 2B is a graph showing the probability of survival of mice administered the indicated antibodies at the indicated doses. mpk=mg per kg of body weight.

FIGS. 3A-3B relate to Example 9. FIG. 3A is a graph showing average tumor volume in mice administered the indicated antibodies at the indicated doses. FIG. 3B is a graph showing the probability of survival of mice administered the indicated antibodies at the indicated doses. mpk=mg per kg of body weight.

FIG. 4 relates to Example 10 and is a graph showing average radiance of mice administered the indicated antibodies.

FIG. 5A-5E relate to Example 11. FIG. 5A is a graph showing average tumor volume in mice administered the indicated antibodies. FIGS. 5B-5E are graphs showing tumor volume in individual mice administered specific antibodies. FIG. 5B shows tumor volume in individual mice administered Isotype control. FIG. 5C shows tumor volume in individual mice administered REGN6191. FIG. 5D shows tumor volume in individual mice administered anti-PD1. FIG. 5E shows tumor volume in individual mice administered REGN6191+anti-PD1.

FIGS. 6A-6D relate to Example 12. FIG. 6A is a graph showing the concentration at Day 0 of IL-2 in the serum of mice administered the indicated antibodies. FIG. 6B is a graph showing the concentration at Day 7 of IFNg in the serum of mice administered the indicated antibodies. FIG. 6C is a graph showing the concentration at Day 7 of ALT in the serum of mice administered the indicated antibodies. FIG. 6D is a graph showing the concentration at Day 7 of IL-2 in the serum of mice administered the indicated antibodies.

FIGS. 7A-7C relate to Example 13 and are graphs showing tumor volume in individual mice administered the indicated antibodies at the indicated doses. FIG. 7A shows tumor volume in individual mice administered 0.01 mg/kg EGFR×CD3. FIG. 7B shows tumor volume in individual mice administered 0.01 mg/kg EGFR×CD3+0.1 mg/kg PD-L1×BetV1. FIG. 7C shows tumor volume in individual mice administered 0.01 mg/kg EGFR×CD3+0.1 mg/kg PD-L1×4-1BB. “mpk” is mg/kg.

### DETAILED DESCRIPTION

Before the present disclosure is described, it is to be understood that this disclosure is not limited to particular methods and experimental conditions described, as such methods and conditions may vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice or testing of the present disclosure, the preferred methods and materials are now described.

The PD-L1 binding arm of the bispecific antibody acts like a bridge to anchor 4-1BB-activating antibodies to tumor, providing a pan-tumor costimulatory approach (tumor agnostic) to convert an inhibitory checkpoint (PD-L1) widely present across various tumor indications and turn it into a costimulatory signal to activate 4-1BB on T cells.

Just as CAR-T approaches have employed chimeric receptors that artificially activate both “signal 1” and “signal 2” so as to improve their anti-tumor activity, there is potential benefit of combining PD-1 inhibition with 4-1BB-agonism (which provide “signal 2”) to enhance anti-tumor activity. This approach has several practical benefits over CAR-T therapies in that it does not require a laborious cell therapy preparation that must be individually customized for each patient, nor does it require that patients be pre-emptively “lymphodepleted” via toxic chemotherapy that is often associated with adverse effects so that they can't accept cell therapy. This bispecific approach offers the potential for increased efficacy as well as increased safety through its specificity of action. Collectively, the data here suggest that combining 4-1BB-agonism with blockade of the inhibitory checkpoint ligand PD-L1 may provide well-tolerated, biologics solutions with markedly enhanced and synergistic anti-tumor activity.

### Definitions

“PD-L1” and “PD-L1 fragment,” as used herein refer to the human PD-L1 protein (also known as CD274, B7-H, B7H1, PDCD1L1, and PDCD1LG1) or a fragment thereof unless specified as being from a non-human species (e.g., “mouse PD-L1,” “mouse PD-L1 fragment,” “monkey PD-L1,” “monkey PD-L1 fragment,” etc.). In an embodiment of the disclosure, human PD-L1 comprises the amino acid sequence set forth in NCBI accession no. AAH69381.1. In one embodiment, a human PD-L1 fragment is shown with a C-terminal myc-myc-hexahistidine tag (hPD-L1.mmH) (SEQ ID NO: 79).

“4-1BB,” as used herein, refers to the human 4-1BB protein (also known as CD137 or TNFRS9) which is expressed on T cells as a costimulatory receptor unless specified as being from a non-human species. In an embodiment of the disclosure, human 4-1BB comprises the amino acid sequence as set forth NCBI accession No. AAA53133.1. In one embodiment, a human 4-1BB fragment is expressed with a C-terminal myc-myc-hexahistidine tag (hCD137 (4-1BB) mmH, SEQ ID NO: 80).

“Isolated” antigen-binding proteins (e.g., antibodies or antigen-binding fragments thereof), polypeptides, polynucleotides and vectors, are at least partially free of other biological molecules from the cells or cell culture from which they are produced. Such biological molecules include nucleic acids, proteins, other antibodies or antigen-binding fragments, lipids, carbohydrates, or other material such as cellular debris and growth medium. An isolated antigen-binding protein may further be at least partially free of expression system components such as biological molecules from a host cell or of the growth medium thereof. Generally, the term “isolated” is not intended to refer to a complete absence of such biological molecules or to an absence of water, buffers, or salts or to components of a pharmaceutical formulation that includes the antigen-binding proteins (e.g., antibodies or antigen-binding fragments).

The following references relate to BLAST algorithms often used for sequence analysis: BLAST ALGORITHMS: Altschul et al. (2005) FEBS J. 272 (20): 5101-5109; Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, D.C.; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, D.C.; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, N.Y.

An “antibody” is an immunoglobulin molecule comprising four polypeptide chains, two heavy chains (HC) and two light chains (LC) inter-connected by disulfide bonds. Each heavy chain (HC) comprises a heavy chain variable region (abbreviated herein as HCVR or V_{H}) and a heavy chain constant region (e.g., IgG, IgG1 or IgG4). The heavy chain constant region comprises three domains, C_{H}1, C_{H}2 and C_{H}3. Each light chain (LC) comprises a light chain variable region (abbreviated herein as LCVR or V_{L}) and a light chain constant region (e.g., lambda or kappa). The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} includes three CDRs and four FRs, arranged from amino-terminus to carboxy-terminus in the following order: FR1, CDR1, FR2, CDR2, FR3, CDR3, FR4. A heavy chain CDR may be referred to as HCDR and a light chain CDR may be referred to as LCDR. In different embodiments of the disclosure, the FRs of an antibody (or antigen-binding portion thereof) may be identical to the human germline sequences, or may be naturally or artificially modified.

An antigen-binding arm of a Y-shaped IgG antibody (e.g., a 4-1BB or PD-L1 binding arm) refers to a structural portion of the antibody that confers binding specificity to the antigen. For example, an antigen-binding arm of an IgG antibody has a heavy chain (HC) associated with a light chain (LC).

An antibody which, for example, is bispecific includes an arm (or domain) that binds to a first antigen and another arm (or domain) that binds to a second antigen. For example, an PD-L1×4-1BB bispecific antibody includes one arm that binds PD-L1 and another arm that binds to 4-1BB.

Bispecific antigen-binding molecules (e.g., bispecific antibodies) may have an effector arm that binds to a first antigen and a targeting arm that binds to second antigen. The effector arm may be the first antigen-binding domain (e.g., anti-4-1BB) that binds to the antigens on effector cells (e.g., T cells). The targeting arm may be the second antigen-binding domain that binds to an antigen on target cells (e.g., tumor cells or immune cells). In the context of the present disclosure, the effector arm binds to 4-1BB and the targeting arm binds to the inhibitory checkpoint ligand PD-L1.

An “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. A multispecific antigen-binding fragment of an antibody binds to multiple antigens (e.g., two different antigens if the fragment is bispecific). Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Non-limiting examples of antigen-binding fragments include: (i) Fab fragments; (ii) F(ab′)₂ fragments; (iii) Fd fragments; (iv) Fv fragments; (v) single-chain Fv (scFv) molecules; and (vi) dAb fragments.

An antigen-binding fragment of an antibody will, in an embodiment of the disclosure, comprise at least one variable domain. The variable domain may be of any size or amino acid composition and will generally comprise at least one CDR, which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) V_{H}-C_{H}1; (ii) V_{H}-C_{H}2; (iii) V_{H}-C_{H}3; (IV) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (Xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids, which result in a flexible or semi-flexible linkage between adjacent variable and/or constant domains in a single polypeptide molecule. Moreover, an antigen-binding fragment of an antibody of the present disclosure may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

The term “recombinant” antigen-binding proteins, such as antibodies or antigen-binding fragments thereof, refers to such molecules created, expressed, isolated or obtained by technologies or methods known in the art as recombinant DNA technology which include, e.g., DNA splicing and transgenic expression. The term includes antibodies expressed in a non-human mammal (including transgenic non-human mammals, e.g., transgenic mice), or a host cell (e.g., Chinese hamster ovary (CHO) cell) or cellular expression system or isolated from a recombinant combinatorial human antibody library. The present disclosure includes recombinant antigen-binding proteins as set forth herein.

The term “specifically binds” or “binds specifically” refers to those antigen-binding proteins (e.g., antibodies or antigen-binding fragments thereof) having a binding affinity to an antigen, such as PD-L1 or 4-1BB protein, expressed as K_{D}, of less than about 10⁻⁶ M (e.g., 10⁻⁷ M, 10⁻⁸ M, 10⁻⁹ M, 10⁻¹⁰ M, 10⁻¹¹ M or 10⁻¹² M), as measured by real-time, label free bio-layer interferometry assay, for example, at 25° C. or 37° C., e.g., an Octet® HTX biosensor, or by surface plasmon resonance, e.g., BIACORE™, or by solution-affinity ELISA. “Anti-PD-L1” refers to an antigen-binding protein (or other molecule such as an antigen-binding arm), for example an antibody or antigen-binding fragment thereof, that binds specifically to PD-L1 and “anti-4-1BB” refers to an antigen-binding protein (or other molecule such as an antigen-binding arm), for example an antibody or antigen-binding fragment thereof, that binds specifically to 4-1BB. “PD-L1×4-1BB” refers to refers to an antigen-binding protein (or other molecule), for example an antibody or antigen-binding fragment thereof, that binds specifically to PD-L1 and to 4-1BB.

The present disclosure includes antigen-binding proteins, e.g., antibodies or antigen-binding fragments, that bind to the same PD-L1 and 4-1BB epitopes as an antigen-binding protein of the present disclosure (e.g., REGN6191).

The term “epitope” refers to an antigenic determinant (e.g., on PD-L1 or 4-1BB) that interacts with a specific antigen-binding site of an antigen-binding protein, e.g., a variable region of an antibody molecule, known as a paratope. A single antigen may have more than one epitope. Thus, different antibodies may bind to different areas on an antigen and may have different biological effects. The term “epitope” may also refer to a site on an antigen to which B and/or T cells respond and/or to a region of an antigen that is bound by an antibody. Epitopes may be defined as structural or functional. Functional epitopes are generally a subset of the structural epitopes and have those residues that directly contribute to the affinity of the interaction. Epitopes may be linear or conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics.

Methods for determining the epitope of an antigen-binding protein, e.g., antibody or fragment or polypeptide, include alanine scanning mutational analysis, peptide blot analysis (Reineke (2004) Methods Mol. Biol. 248:443-63), peptide cleavage analysis, crystallographic studies and NMR analysis. In addition, methods such as epitope excision, epitope extraction and chemical modification of antigens can be employed (Tomer (2000) Prot. Sci. 9:487-496). Another method that can be used to identify the amino acids within a polypeptide with which an antigen-binding protein (e.g., antibody or fragment or polypeptide) interacts is hydrogen/deuterium exchange detected by mass spectrometry. See, e.g., Ehring (1999) Analytical Biochemistry 267:252-259; Engen and Smith (2001) Anal. Chem. 73: 256A-265A.

The present disclosure includes antigen-binding proteins that compete for binding to 4-1BB and PD-L1 with an antigen-binding protein of the present disclosure, e.g., REGN6188. The term “competes” as used herein, refers to an antigen-binding protein (e.g., antibody or antigen-binding fragment thereof) that binds to an antigen and inhibits or blocks the binding of another antigen-binding protein (e.g., antibody or antigen-binding fragment thereof) to the antigen. Unless otherwise stated, the term also includes competition between two antigen-binding proteins e.g., antibodies, in both orientations, i.e., a first antibody that binds antigen and blocks binding by a second antibody and vice versa. Thus, in an embodiment of the disclosure, competition occurs in one such orientation. In certain embodiments, the first antigen-binding protein (e.g., antibody) and second antigen-binding protein (e.g., antibody) may bind to the same epitope. Alternatively, the first and second antigen-binding proteins (e.g., antibodies) may bind to different, but, for example, overlapping or non-overlapping epitopes, wherein binding of one inhibits or blocks the binding of the second antibody, e.g., via steric hindrance. Competition between antigen-binding proteins (e.g., antibodies) may be measured by methods known in the art, for example, by a real-time, label-free bio-layer interferometry assay. Also, binding competition between antigen-binding proteins (e.g., monoclonal antibodies (mAbs)) can be determined using a real time, label-free bio-layer interferometry assay on an Octet RED384 biosensor (Pall ForteBio Corp.).

Typically, an antibody or antigen-binding fragment of the disclosure which is modified in some way retains the ability to specifically bind to PD-L1 and 4-1BB, e.g., retains at least 10% of its PD-L1 and 4-1BB binding activity (when compared to the parental antibody) when that activity is expressed on a molar basis. Preferably, an antibody or antigen-binding fragment of the disclosure retains at least 20%, 50%, 70%, 80%, 90%, 95% or 100% or more of the PD-L1 and 4-1BB binding affinity as the parental antibody. It is also intended that an antibody or antigen-binding fragment of the disclosure may include conservative or non-conservative amino acid substitutions (referred to as “conservative variants” or “function conserved variants” of the antibody) that do not substantially alter its biologic activity.

A “variant” of a polypeptide, such as an immunoglobulin chain (e.g., a chain of REGN6191) V_{H}, V_{L}, HC or LC or CDR thereof comprising the amino acid sequence specifically set forth herein), refers to a polypeptide comprising an amino acid sequence that is at least about 70-99.9% (e.g., at least 70, 72, 74, 75, 76, 79, 80, 81, 82, 83, 84, 85, 86, 87, 88, 89, 90, 91, 92, 93, 94, 95, 96, 97, 98, 99, 99.5 or 99.9%) identical or similar to a referenced amino acid sequence that is set forth herein (e.g., any of SEQ ID NOs: 2, 4, 6; 8; 10; 12; 14; 16; 18; 20; 22; 24; 26; 28; 30; 32; 34; 36; 38; 40; 42; 44; 46; 48; 50; 52; 54; 56; 58; 60; 62; 64; 66; 68; 70; 72; 74; 76 or 78); when the comparison is performed by a BLAST algorithm wherein the parameters of the algorithm are selected to give the largest match between the respective sequences over the entire length of the respective reference sequences (e.g., expect threshold: 10; word size: 3; max matches in a query range: 0; BLOSUM 62 matrix; gap costs: existence 11, extension 1; conditional compositional score matrix adjustment).

Moreover, a variant of a polypeptide may include a polypeptide such as an immunoglobulin chain (e.g., a chain of REGN6191) V_{H}, V_{L}, HC or LC or CDR thereof) which may include the amino acid sequence of the reference polypeptide whose amino acid sequence is specifically set forth herein but for one or more (e.g., 1, 2, 3, 4, 5, 6, 7, 8, 9 or 10) mutations, e.g., one or more missense mutations (e.g., conservative substitutions), non-sense mutations, deletions, or insertions. For example, the present disclosure includes 4-1BB×PD-L1 antigen-binding proteins which include an PD-L1 binding arm immunoglobulin light chain (or V_{L}) variant comprising the amino acid sequence set forth in SEQ ID NO: 18 but having one or more of such mutations and/or an immunoglobulin heavy chain (or V_{H}) variant comprising the amino acid sequence set forth in SEQ ID NO: 2 but having one or more of such mutations. In an embodiment of the disclosure, a 4-1BB×PD-L1 antigen-binding protein includes an immunoglobulin light chain variant comprising LCDR1, LCDR2 and LCDR3 wherein one or more (e.g., 1 or 2 or 3) of such CDRs has one or more of such mutations (e.g., conservative substitutions) and/or an immunoglobulin heavy chain variant comprising HCDR1, HCDR2 and HCDR3 wherein one or more (e.g., 1 or 2 or 3) of such CDRs has one or more of such mutations (e.g., conservative substitutions).

A “conservatively modified variant” or a “conservative substitution”, e.g., of an immunoglobulin chain set forth herein, refers to a variant wherein there is one or more substitutions of amino acids in a polypeptide with other amino acids having similar characteristics (e.g., charge, side-chain size, hydrophobicity/hydrophilicity, backbone conformation and rigidity, etc.). Such changes can frequently be made without significantly disrupting the biological activity of the antibody or fragment. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4^{th} Ed.)). In addition, substitutions of structurally or functionally similar amino acids are less likely to significantly disrupt biological activity. The present disclosure includes PD-L1×4-1BB antigen-binding proteins and/or binding arms comprising such conservatively modified variant immunoglobulin chains.

Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. Alternatively, a conservative replacement is any change having a positive value in the PAM250 log-likelihood matrix disclosed in Gonnet et al. (1992) Science 256:1443-45.

As used herein, the singular forms “a,” “an,” and “the” include plural reference unless the context clearly dictates otherwise. As used herein, the terms “including,” “comprising,” “containing,” or “having” and variations thereof are meant to encompass the items listed thereafter and equivalents thereof as well as additional subject matter unless otherwise noted. As used herein, the phrases “in one embodiment,” “in various embodiments,” “in some embodiments,” and the like are used repeatedly. Such phrases do not necessarily refer to the same embodiment, but they may unless the context dictates otherwise. As used herein, the terms “and/or” or “/” means any one of the items, any combination of the items, or all of the items with which this term is associated.

### PD-L1×4-1BB Antigen-Binding Molecules, Antibodies and Antigen-Binding Fragments Thereof

The antibodies or antigen-binding molecules of the present disclosure may be bi-specific, or multispecific. Multispecific antibodies or antigen-binding molecules may be specific for different epitopes of one target polypeptide or may contain antigen-binding domains specific for more than one target polypeptide. See, e.g., Tutt et al., 1991, J. Immunol. 147:60-69; Kufer et al., 2004, Trends Biotechnol. 22:238-244. The antibodies of the present disclosure can be linked to or co-expressed with another functional molecule, e.g., another peptide or protein. For example, an antibody or fragment thereof can be functionally linked (e.g., by chemical coupling, genetic fusion, noncovalent association or otherwise) to one or more other molecular entities, such as another antibody or antibody fragment to produce a bi-specific or a multispecific antibody with a second binding specificity.

Use of the expression “anti-4-1BB antibody” herein is intended to include both monospecific anti-4-1BB antibodies as well as multispecific (e.g., bispecific) antibodies comprising a 4-1BB-binding arm and a second arm that binds a target antigen. Thus, the present disclosure includes bispecific antibodies wherein one arm of an immunoglobulin binds human 4-1BB, and the other arm of the immunoglobulin is specific for a target antigen. The target antigen that the other arm of the 4-1BB bispecific antibody binds can be any antigen expressed on or in the vicinity of a cell, tissue, organ, microorganism or virus, against which a targeted immune response is desired. The 4-1BB-binding arm can comprise any of the HCVR/LCVR or CDR amino acid sequences as set forth in Tables 3 and 8 herein. In certain embodiments, the 4-1BB-binding arm binds human 4-1BB and induces human T-cell proliferation.

According to certain exemplary embodiments, the present disclosure includes bispecific antigen-binding molecules that specifically bind 4-1BB and PD-L1. Such molecules may be referred to herein as, e.g., “anti-4-1BB/anti-PD-L1,” or “anti-4-1BB×PD-L1,” or “4-1BB×PD-L1” or “PD-L1×4-1BB”, or “anti-PD-L1/anti-4-1BB,” or “anti-PD-L1×4-1BB,” or “PD-L1×4-1BB” bispecific molecules, or “anti-PD-L1×anti-4-1BB” or “anti-4-1BB×anti-PD-L1”, or other similar terminology.

According to certain exemplary embodiments, the bispecific antigen-binding molecules (e.g., bispecific antibody) may have an effector arm and a targeting arm. The effector arm may be the first antigen-binding domain (e.g., anti-4-1BB antibody) that binds to the antigens on effector cells (e.g., T cells). The targeting arm may be the second antigen-binding domain (e.g., anti-/PD-L1 antibody) that binds to an antigen on target cells (e.g., tumor cells or antigen-presenting cells). According to certain exemplary embodiments, the effector arm binds to 4-1BB and the targeting arm binds to the inhibitory checkpoint ligand PD-L1. The bispecific anti-4-1BB/PD-L1 may provide a pan-tumor costim approach (tumor agnostic) to convert an inhibitory checkpoint (PD-L1) widely present across various tumor indications and turn it into a costimulatory signal to activate 4-1BB on T cells.

As used herein, the expression “antigen-binding molecule” means a protein, polypeptide or molecular complex comprising or consisting of at least one complementarity determining region (CDR) that alone, or in combination with one or more additional CDRs and/or framework regions (FRs), specifically binds to a particular antigen. In certain embodiments, an antigen-binding molecule is an antibody or a fragment of an antibody, as those terms are defined elsewhere herein.

As used herein, the expression “bispecific antigen-binding molecule” means a protein, polypeptide or molecular complex (e.g., an antibody or antigen-binding fragment thereof) comprising at least a first antigen-binding domain and a second antigen-binding domain. Each antigen-binding domain within the bispecific antigen-binding molecule comprises at least one CDR that alone, or in combination with one or more additional CDRs and/or FRs, specifically binds to a particular antigen. In the context of the present disclosure, the first antigen-binding domain specifically binds a first antigen (e.g., 4-1BB), and the second antigen-binding domain specifically binds a second, distinct antigen (e.g., PD-L1).

In certain exemplary embodiments of the present disclosure, the bispecific antigen-binding molecule is a bispecific antibody. Each antigen-binding domain of a bispecific antibody comprises a heavy chain variable domain (HCVR) and a light chain variable domain (LCVR).

The first antigen-binding domain and the second antigen-binding domain may be directly or indirectly connected to one another to form a bispecific antigen-binding molecule of the present disclosure. Alternatively, the first antigen-binding domain and the second antigen-binding domain may each be connected to a separate multimerizing domain. The association of one multimerizing domain with another multimerizing domain facilitates the association between the two antigen-binding domains, thereby forming a bispecific antigen-binding molecule. As used herein, a “multimerizing domain” is any macromolecule, protein, polypeptide, peptide, or amino acid that has the ability to associate with a second multimerizing domain of the same or similar structure or constitution. For example, a multimerizing domain may be a polypeptide comprising an immunoglobulin C_{H}3 domain. A non-limiting example of a multimerizing component is an Fc portion of an immunoglobulin (comprising a C_{H}2-C_{H}3 domain), e.g., an Fc domain of an IgG selected from the isotypes IgG1, IgG2, IgG3, and IgG4, as well as any allotype within each isotype group. The Fc domain may comprise wild-type or modified IgG isotype.

Bispecific antigen-binding molecules of the present disclosure will typically comprise two multimerizing domains, e.g., two Fc domains that are each individually part of a separate antibody heavy chain. The first and second multimerizing domains may be of the same IgG isotype such as, e.g., IgG1/IgG1, IgG2/IgG2, IgG4/IgG4. Alternatively, the first and second multimerizing domains may be of different IgG isotypes such as, e.g., IgG1/IgG2, IgG1/IgG4, IgG2/IgG4, etc.

In certain embodiments, the multimerizing domain is an Fc fragment or an amino acid sequence of 1 to about 200 amino acids in length containing at least one cysteine residues. In other embodiments, the multimerizing domain is a cysteine residue, or a short cysteine containing peptide. Other multimerizing domains include peptides or polypeptides comprising or consisting of a leucine zipper, a helix-loop motif, or a coiled-coil motif.

Any bispecific antibody format or technology may be used to make the bispecific antigen-binding molecules of the present disclosure. For example, an antibody or antigen-binding fragment thereof having a first antigen-binding specificity can be functionally linked (e.g., by chemical coupling, genetic fusion, noncovalent association or otherwise) to one or more other molecular entities, such as another antibody or antibody fragment having a second antigen-binding specificity to produce a bispecific antigen-binding molecule. Specific exemplary bispecific formats that can be used in the context of the present disclosure include, without limitation, e.g., scFv-based or diabody bispecific formats, IgG-scFv fusions, dual variable domain (OVO)-Ig, Quadroma, knobs-into-holes, common light chain (e.g., common light chain with knobs-intoholes, etc.), CrossMab, CrossFab, (SEEO) body, leucine zipper, Ouobody, IgG1/IgG2, dual acting Fab (OAF)-IgG, and Mab² bispecific formats (see, e.g., Klein et al. 2012, mAbs 4:6, 1-11, and references cited therein, for a review of the foregoing formats).

In the context of bispecific antigen-binding molecules of the present disclosure, the multimerizing domains, e.g., Fc domains, may comprise one or more amino acid changes (e.g., insertions, deletions or substitutions) as compared to the wild-type, naturally occurring version of the Fc domain. For example, the disclosure includes bispecific antigen-binding molecules comprising one or more modifications in the Fc domain that results in a modified Fc domain having a modified binding interaction (e.g., enhanced or diminished) between Fc and FcRn. In one embodiment, the bispecific antigen-binding molecule comprises a modification in a C_{H}2 or a C_{H}3 region, wherein the modification increases the affinity of the Fc domain to FcRn in an acidic environment (e.g., in an endosome where pH ranges from about 5.5 to about 6.0). Non-limiting examples of such Fc modifications include, e.g., a modification at position 250 (e.g., E or Q); 250 and 428 (e.g., L or F); 252 (e.g., LN/FIW or T), 254 (e.g., S or T), and 256 (e.g., S/R/Q/EID or T); or a modification at position 428 and/or 433 (e.g., UR/S/P/Q or K) and/or 434 (e.g., H/F or V); or a modification at position 250 and/or 428; or a modification at position 307 or 308 (e.g., 308F, V308F), and 434. In one embodiment, the modification comprises a 428L (e.g., M428L) and 434S (e.g., N434S) modification; a 428L, 2591 (e.g., V2591), and 308F (e.g., V308F) modification; a 433K (e.g., H433K) and a 434 (e.g., 434Y) modification; a 252,254, and 256 (e.g., 252Y, 254T, and 256E) modification; a 250Q and 428L modification (e.g., T250Q and M428L); and a 307 and/or 308 modification (e.g., 308F or 308P).

The present disclosure also includes bispecific antigen-binding molecules comprising a first C_{H}3 domain and a second Ig C_{H}3 domain, wherein the first and second Ig C_{H}3 domains differ from one another by at least one amino acid, and wherein at least one amino acid difference reduces binding of the bispecific antibody to Protein A as compared to a bi-specific antibody lacking the amino acid difference. In one embodiment, the first Ig C_{H}3 domain binds Protein A and the second Ig C_{H}3 domain contains a mutation that reduces or abolishes Protein A binding such as an H95R modification (by IMGT exon numbering; H435R by EU numbering). The second C_{H}3 may further comprise a Y96F modification (by IMGT; Y436F by EU). Further modifications that may be found within the second C_{H}3 include: D16E, L 18M, N44S, K52N, V57M, and V821 (by IMGT; D356E, L358M, N384S, K392N, V397M, and V4221 by EU) in the case of IgG1 antibodies; N44S, K52N, and V821 (IMGT; N384S, K392N, and V4221 by EU) in the case of IgG2 antibodies; and Q15R, N44S, K52N, V57M, R69K, E79Q, and V821 (by IMGT; Q355R, N384S, K392N, V397M, R409K, E419Q, and V4221 by EU) in the case of IgG4 antibodies.

In certain embodiments, the Fc domain may be chimeric, combining Fc sequences derived from more than one immunoglobulin isotype. For example, a chimeric Fc domain can comprise part or all of a C_{H}2 sequence derived from a human IgG1, human IgG2 or human IgG4 C_{H}2 region, and part or all of a C_{H}3 sequence derived from a human IgG1, human IgG2 or human IgG4. A chimeric Fc domain can also contain a chimeric hinge region. For example, a chimeric hinge may comprise an “upper hinge” sequence, derived from a human IgG1, a human IgG2 or a human IgG4 hinge region, combined with a “lower hinge” sequence, derived from a human IgG1, a human IgG2 or a human IgG4 hinge region. A particular example of a chimeric Fc domain that can be included in any of the antigen-binding molecules set forth herein comprises, from N- to C-terminus: [IgG4 C_{H}1]-[IgG4 upper hinge]-[IgG2 lower hinge]-[IgG4 C_{H}2]-[IgG4 C_{H}3]. Another example of a chimeric Fc domain that can be included in any of the antigen-binding molecules set forth herein comprises, from N- to C-terminus: [IgG1 C_{H}1]-[IgG1 upper hinge]-[IgG2 lower hinge]-[IgG4 C_{H}2]-[IgG1 C_{H}3]. These and other examples of chimeric Fc domains that can be included in any of the antigen-binding molecules of the present disclosure are described in WO2014/022540 A1, Chimeric Fc domains having these general structural arrangements, and variants thereof, can have altered Fc receptor binding, which in turn affects Fc effector function.

Antibodies and antigen-binding fragments of the present disclosure comprise immunoglobulin chains including the amino acid sequences specifically set forth herein (and variants thereof) as well as cellular and in vitro post-translational modifications to the antibody or fragment. For example, the present disclosure includes antibodies and antigen-binding fragments thereof that specifically bind to PD-L1 and 4-1BB comprising heavy and/or light chain amino acid sequences set forth herein as well as antibodies and fragments wherein one or more asparagine, serine and/or threonine residues is glycosylated, one or more asparagine residues is deamidated, one or more residues (e.g., Met, Trp and/or His) is oxidized, the N-terminal glutamine is pyroglutamate (pyroE) and/or the C-terminal lysine or other amino acid is missing.

The bispecific antigen-binding molecules of the present disclosure comprise a first antigen-binding arm that specifically binds 4-1BB (“4-1BB-binding arm” “4-1BB binding domain”). In certain embodiments, the 4-1BB-binding arm comprises HCVR and LCVR comprising amino acid sequences as disclosed herein. The bispecific antigen-binding molecules also comprise a second antigen-binding arm that specifically binds PD-L1 (“an PD-L1 binding arm” or “PD-L1 binding domain”). In certain embodiments, the PD-L1-binding arm comprises HCVR and LCVR comprising amino acid sequences as disclosed herein. In certain embodiments, the PD-L1-binding arm includes a heavy chain immunoglobulin that comprises a V_{H} including the combination of heavy chain CDRs (HCDR1, HCDR2 and HCDR3) and the corresponding light chain immunoglobulin that comprises a V_{L} including the combination of light chain CDRs (LCDR1, LCDR2 and LCDR3) which are set forth herein or in WO2014/004427.

The present disclosure includes multispecific (e.g., bispecific) antigen-binding proteins, e.g., antibodies or antigen-binding fragments, including one or more 4-1BB Binding Arms as well as one or more PD-L1 Binding Arms.

A 4-1BB Binding Arm is the portion of a multispecific antigen-binding protein that confers 4-1BB binding upon the protein. For example, a 4-1BB-binding arm of a Y-shaped IgG antibody refers to a structural portion of the antibody that confers binding specificity to the 4-1BB. For example, in an embodiment of the disclosure, a 4-1BB Binding Arm comprises HCDR1, LCDR1, HCDR2, LCDR2, HCDR3 and LCDR3; a HVCR (V_{H}) and an LCVR (V_{L}) and/or a HC and LC that binds specifically to 4-1BB.

In an embodiment of the disclosure, a 4-1BB Binding Arm includes a heavy chain immunoglobulin that comprises a V_{H} including the combination of heavy chain CDRs (HCDR1, HCDR2 and HCDR3) and the corresponding light chain immunoglobulin that comprises a V_{L} including the combination of light chain CDRs (LCDR1, LCDR2 and LCDR3) which are set forth herein. In an embodiment of the disclosure, a 4-1BB Binding Arm includes a heavy chain variable region (V_{H}) and the corresponding light chain variable region (V_{L}) set forth herein.

The bispecific PD-L1×4-1BB antigen-binding proteins of the present disclosure which bind to 4-1BB on the surface of a T-cell and agonize 4-1BB signaling enhancing activation and/or proliferation of the T-cell may be referred to, herein, as “co-stimulatory” or “costimulatory”. T-cell activation is initiated upon binding of the T-cell Receptor (TCR)/CD3 complex to peptide-MHC complexes (“signal 1”); activation is then enhanced by engagement of a second “co-stimulatory” receptor, such as the 4-1BB receptor on T-cells binding to its cognate ligand(s) on the target cell (“signal 2”). For example, activation of a T-cell by a 4-1BB bi-specific antibody may be caused by amplification of signals in response to endogenous tumor antigen recognition by the TCR/CD3 complex, or in response to “signal 1” activation via a CD3-bispecific.

### Polynucleotides and Methods of Making

An isolated polynucleotide molecule or a set of polynucleotide molecules comprising polynucleotide sequences encoding the immunoglobulin chains of any PD-L1×4-1BB bispecific antigen-binding molecule set forth herein forms part of the present disclosure. The present disclosure also includes a vector or a set of vectors comprising the polynucleotide molecules and/or a host cell (e.g., Chinese hamster ovary (CHO) cell) comprising the polynucleotide molecules, vector(s) or antigen-binding protein set forth herein.

A polynucleotide molecule or sequence includes DNA and RNA. The present disclosure includes any polynucleotide molecule or sequence of the present disclosure, for example, encoding an immunoglobulin V_{H}, V_{L}, CDR-H, CDR-L, HC or LC of an PD-L1 Binding Arm and/or a 4-1BB Binding Arm, optionally, which is operably linked to a promoter or other expression control sequence. For example, the present disclosure provides any polynucleotide (e.g., DNA) that includes a nucleotide sequence set forth in Table 2 and a nucleotide sequence set forth in Table 4.

The present disclosure includes a polynucleotide comprising a nucleotide sequence set forth in SEQ ID NO: 1, 3, 5, 7, 9, 11, 13, 15, 17, 19, 21, 29, 31, 33, 35, 39, 41, 43, 45, 49, 51, 53, 55, 57, 59, 61, 63, 65, 67, 69, 71, 73, 75, and/or 77, optionally operably linked to a promoter or other expression control sequence or other polynucleotide sequence.

In general, a “promoter” or “promoter sequence” is a DNA regulatory region capable of binding an RNA polymerase in a cell (e.g., directly or through other promoter-bound proteins or substances) and initiating transcription of a coding sequence. A promoter may be operably linked to other expression control sequences, including enhancer and repressor sequences and/or with a polynucleotide of the disclosure. Promoters which may be used to control gene expression include, but are not limited to, cytomegalovirus (CMV) promoter (U.S. Pat. Nos. 5,385,839 and 5,168,062), the SV40 early promoter region (Benoist, et al., (1981) Nature 290:304-310), the promoter contained in the 3′ long terminal repeat of Rous sarcoma virus (Yamamoto, et al., (1980) Cell 22:787-797), the herpes thymidine kinase promoter (Wagner, et al., (1981) Proc. Natl. Acad. Sci. USA 78:1441-1445), the regulatory sequences of the metallothionein gene (Brinster, et al., (1982) Nature 296:39-42); prokaryotic expression vectors such as the beta-lactamase promoter (Villa-Komaroff, et al., (1978) Proc. Natl. Acad. Sci. USA 75:3727-3731), or the tac promoter (DeBoer, et al., (1983) Proc. Natl. Acad. Sci. USA 80:21-25); see also “Useful proteins from recombinant bacteria” in Scientific American (1980) 242:74-94; and promoter elements from yeast or other fungi such as the Gal4 promoter, the ADC (alcohol dehydrogenase) promoter, PGK (phosphoglycerol kinase) promoter or the alkaline phosphatase promoter.

A polynucleotide encoding a polypeptide is “operably linked” to a promoter or other expression control sequence when, in a cell or other expression system, the sequence directs RNA polymerase mediated transcription of the coding sequence into RNA, preferably mRNA, which then may be RNA spliced (if it contains introns) and, optionally, translated into a protein encoded by the coding sequence.

The present disclosure includes polynucleotides encoding immunoglobulin polypeptide chains which are variants of those whose nucleotide sequence is specifically set forth herein. A “variant” of a polynucleotide refers to a polynucleotide comprising a nucleotide sequence that is at least about 70-99.9% (e.g., 70, 72, 74, 75, 76, 79, 80, 81, 82, 83, 84, 85, 86, 87, 88, 89, 90, 91, 92, 93, 94, 95, 96, 97, 98, 99, 99.5, 99.9%) identical to a referenced nucleotide sequence that is set forth herein; when the comparison is performed by a BLAST algorithm wherein the parameters of the algorithm are selected to give the largest match between the respective sequences over the entire length of the respective reference sequences (e.g., expect threshold: 10; word size: 28; max matches in a query range: 0; match/mismatch scores: 1, -2; gap costs: linear). In an embodiment of the disclosure, a variant of a nucleotide sequence specifically set forth herein comprises one or more (e.g., 1, 2, 3, 4, 5, 6, 7, 8, 9, 10, 11 or 12) point mutations, insertions (e.g., in frame insertions) or deletions (e.g., in frame deletions) of one or more nucleotides. Such mutations may, in an embodiment of the disclosure, be missense or nonsense mutations. In an embodiment of the disclosure, such a variant polynucleotide encodes an immunoglobulin polypeptide chain which can be incorporated into an PD-L1 Binding Arm and/or 4-1BB Binding Arm, i.e., such that the protein retains specific binding to PD-L1 and/or 4-1BB.

Eukaryotic and prokaryotic host cells, including mammalian cells, may be used as hosts for expression of a PD-L1×4-1BB antigen-binding protein (e.g., antibody or antigen-binding fragment thereof) or an antigen-binding arm thereof. Such host cells are well known in the art and many are available from the American Type Culture Collection (ATCC). These host cells include, inter alia, Chinese hamster ovary (CHO) cells, NSO, SP2 cells, Hela cells, baby hamster kidney (BHK) cells, monkey kidney cells (COS), human hepatocellular carcinoma cells (e.g., Hep G2), A549 cells, 3T3 cells, HEK-293 cells and a number of other cell lines. Mammalian host cells include human, mouse, rat, dog, monkey, pig, goat, bovine, horse and hamster cells. Other cell lines that may be used are insect cell lines (e.g., Spodoptera frugiperda or Trichoplusia ni), amphibian cells, bacterial cells, plant cells and fungal cells. Fungal cells include yeast and filamentous fungus cells including, for example, Pichia, Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Physcomitrella patens and Neurospora crassa. The present disclosure includes an isolated host cell (e.g., a CHO cell or any type of host cell set forth above) comprising an anti-PD-L1×anti-4-1BB antigen-binding protein of the present disclosure, such as REGN6188, REGN6189, REGN6190 and REGN6191 and the anti-PD-L1×anti-4-1BB antigen-binding proteins shown in Table 9, or a polynucleotide encoding an immunoglobulin (Ig) heavy and/or light chain thereof); and/or one or more polynucleotides encoding the PD-L1 binding arm and 4-1BB binding arm of a multispecific antigen-binding protein of the present disclosure.

The present disclosure also includes a cell which is expressing an PD-L1 and/or 4-1BB or an antigenic fragment or fusion thereof (e.g., His₆, Fc and/or myc) which is bound by an PD-L1×4-1BB antigen-binding protein of the present disclosure (e.g., an antibody or antigen-binding fragment thereof), for example, REGN6188, REGN6189, REGN6190 and REGN6191, as well as bispecific antibodies prepared by combining any of the PD-L1 HCVR arms of Table 1 (e.g., HCVR arms of the parental monoclonal antibodies 9364P3 and 9373P2) with any of the 4-1BB HCVR arms of Table 3 (e.g., HCVR arms of the parental 25894P2, 25898P2, 25907P2 and 25921P2), or any of the bispecific antibodies shown in Table 9, for example, wherein the cell is in the body of a subject or is in vitro.

In addition, the present disclosure also provides a complex comprising an PD-L1×4-1BB, antigen-binding protein of the present disclosure, e.g., antibody or antigen-binding fragment thereof, as discussed herein complexed with PD-L1 and/or 4-1BB polypeptide or an antigenic fragment thereof or fusion thereof and/or with a secondary antibody or antigen-binding fragment thereof (e.g., detectably labeled secondary antibody) that binds specifically to the PD-L1×4-1BB antibody or fragment. In an embodiment of the disclosure, the complex is in vitro (e.g., is immobilized to a solid substrate) or is in the body of a subject. In an embodiment of the disclosure, the PD-L1 is on the surface of a tumor cell or antigen presenting cell and the 4-1BB is on the surface of an immune cell, e.g., a T-cell. In an embodiment of the disclosure, the T-cell is activated.

There are several methods by which to produce recombinant antibodies which are known in the art. One example of a method for recombinant production of antibodies is disclosed in U.S. Pat. No. 4,816,567. Transformation can be by any known method for introducing polynucleotides into a host cell. Methods for introduction of heterologous polynucleotides into mammalian cells are well known in the art and include dextran-mediated transfection, calcium phosphate precipitation, polybrene-mediated transfection, protoplast fusion, electroporation, encapsulation of the polynucleotide(s) in liposomes, biolistic injection and direct microinjection of the DNA into nuclei. In addition, nucleic acid molecules may be introduced into mammalian cells by viral vectors. Methods of transforming cells are well known in the art. See, for example, U.S. Pat. Nos. 4,399,216; 4,912,040; 4,740,461 and 4,959,455.

The present disclosure includes recombinant methods for making an anti-PD-L1×anti-4-1BB (e.g., REGN6188, REGN6189, REGN6190 and REGN6191) antigen-binding protein of the present disclosure, such as an antibody or antigen-binding fragment thereof of the present disclosure, or an immunoglobulin chain thereof, comprising
(i) introducing, into a host cell, one or more polynucleotides encoding the light and heavy immunoglobulin chains encoding the PD-L1×4-1BB antigen-binding protein's antigen-binding arms for example, wherein the polynucleotide is in a vector; and/or integrates into the host cell chromosome and/or is operably linked to a promoter;
(ii) culturing the host cell (e.g., CHO or Pichia or Pichia pastoris) under conditions favorable to expression of the polynucleotide and,
(iii) optionally, isolating the antigen-binding protein (e.g., antibody or antigen-binding fragment) or chain from the host cell and/or medium in which the host cell is grown. The present disclosure also includes PD-L1×4-1BB antigen-binding proteins, such as antibodies and antigen-binding fragments thereof, which are the product of the production methods set forth herein, and, optionally, the purification methods set forth herein.

In an embodiment of the disclosure, a method for making an PD-L1×4-1BB (e.g., REGN6188, REGN6189, REGN6190 and REGN6191) antigen-binding protein, e.g., antibody or antigen-binding fragment thereof, includes a method of purifying the antigen-binding protein, e.g., by column chromatography, precipitation and/or filtration. As discussed, the product of such a method also forms part of the present disclosure.

### Sequence Variants

The antibodies and bispecific antigen-binding molecules of the present disclosure may comprise one or more amino acid substitutions, insertions and/or deletions in the framework and/or CDR regions of the heavy and light chain variable domains as compared to the corresponding germline sequences from which the individual antigen-binding domains were derived. Such mutations can be readily ascertained by comparing the amino acid sequences disclosed herein to germ line sequences available from, for example, public antibody sequence databases. The antigen-binding molecules of the present disclosure may comprise antigen-binding fragments which are derived from any of the exemplary amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from which the antigen-binding domain was originally derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germ line sequence from which the antigen-binding domain was originally derived). Furthermore, the antigen-binding domains may contain any combination of two or more germline mutations within the framework and/or CDR regions, e.g., wherein certain individual residues are mutated to the corresponding residue of a particular germ line sequence while certain other residues that differ from the original germ line sequence are maintained or are mutated to the corresponding residue of a different germline sequence. Once obtained, antigen-binding domains that contain one or more germline mutations can be easily tested for one or more desired properties such as improved binding specificity, increased binding affinity, improved or enhanced antagonistic or agonistic biological properties (as the case may be), reduced immunogenicity, etc. Bispecific antigen-binding molecules comprising one or more antigen-binding domains obtained in this general manner are encompassed within the present disclosure.

The present disclosure also includes antigen-binding molecules wherein one or both antigen-binding domains comprise variants of any of the HCVR, LCVR, and/or CDR amino acid sequences disclosed herein having one or more conservative substitutions. For example, the present disclosure includes antigen-binding molecules comprising an antigen-binding domain having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256:1443-1445. A “moderately conservative” replacement is any change having a nonnegative value in the PAM250 log-likelihood matrix.

The present disclosure also includes antigen-binding molecules comprising an antigen-binding domain with an HCVR, LCVR, and/or CDR amino acid sequence that is substantially identical to any of the HCVR, LCVR, and/or CDR amino acid sequences disclosed herein. The term “substantial identity” or “substantially identical,” when referring to an amino acid sequence means that two amino acid sequences, when optimally aligned, such as by the programs GAP or BESTFIT using default gap weights, share at least 95% sequence identity, even more preferably at least 98% or 99% sequence identity. Preferably, residue positions which are not identical differ by conservative amino acid substitutions. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24:307-331.

Sequence similarity for polypeptides, which is also referred to as sequence identity, is typically measured using sequence analysis software. Protein analysis software matches similar sequences using measures of similarity assigned to various substitutions, deletions and other modifications, including conservative amino acid substitutions. For instance, GCG software contains programs such as Gap and Bestfit which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (Pearson (2000) Methods Mol. Biol. 132:185-219). Another preferred algorithm when comparing a sequence of the disclosure to a database containing a large number of sequences from different organisms is the computer program BLAST, especially BLASTP or TBLASTN, using default parameters. See, e.g., Altschul et al. (1990) J. Mol. Biol. 215:403-410; Altschul et al. (1997) Nucleic Acids Res. 25:3389-402.

### Antibodies Comprising Fc Variants

According to certain embodiments of the present disclosure, anti-PD-L1×anti-4-1BB bispecific antigen-binding molecules are provided comprising an Fc domain comprising one or more mutations which enhance or diminish antibody binding to the FcRn receptor, e.g., at acidic pH as compared to neutral pH. For example, the present disclosure includes antibodies and antigen-binding molecules comprising a mutation in the C_{H}2 or a C_{H}3 region of the Fc domain, wherein the mutation(s) increases the affinity of the Fc domain to FcRn in an acidic environment (e.g., in an endosome where pH ranges from about 5.5 to about 6.0). Such mutations may result in an increase in serum half-life of the antibody when administered to an animal. Non-limiting examples of such Fc modifications include, e.g., a modification at position
250 (e.g., E or Q);
250 and 428 (e.g., L or F);
252 (e.g., L/Y/F/W or T),
254 (e.g., S or T), and/or
256 (e.g., S/R/Q/E/D or T);
   or a modification at position
428 and/or 433 (e.g., H/L/R/S/P/Q or K) and/or
434 (e.g., H/F or Y);
   or a modification at position
250 and/or 428;
   or a modification at position
307 or 308 (e.g., 308F, V308F), and/or
434.

In one embodiment, the modification comprises a
428L (e.g., M428L) and 434S (e.g., N434S) modification;
a 428L, 2591 (e.g., V2591), and 308F (e.g., V308F) modification;
a 433K (e.g., H433K) and a 434 (e.g., 434Y) modification;
a 252, 254, and 256 (e.g., 252Y, 254T, and 256E) modification;
a 250Q and 428L modification (e.g., T250Q and M428L); and/or
a 307 and/or 308 modification (e.g., 308F or 308P).

For example, the present disclosure includes PD-L1×4-1BB bispecific antigen-binding molecules comprising an Fc domain comprising one or more pairs or groups of mutations selected from the group consisting of:
250Q and 248L (e.g., T250Q and M248L);
252Y, 254T and 256E (e.g., M252Y, S254T and T256E);
428L and 434S (e.g., M428L and N434S); and
433K and 434F (e.g., H433K and N434F).

The present disclosure also includes bispecific antigen-binding molecules comprising a first C_{H}3 domain and a second Ig C_{H}3 domain, wherein the first and second Ig C_{H}3 domains differ from one another by at least one amino acid, and wherein at least one amino acid difference reduces binding of the bispecific antibody to Protein A as compared to a bi-specific antibody lacking the amino acid difference. In one embodiment, the first Ig C_{H}3 domain binds Protein A and the second Ig C_{H}3 domain contains a mutation that reduces or abolishes Protein A binding such as an H95R modification (by IMGT exon numbering; H435R by EU numbering). The second C_{H}3 may further comprise a Y96F modification (by IMGT; Y436F by EU). See, for example, U.S. Pat. No. 8,586,713. Further modifications that may be found within the second C_{H}3 include: D16E, L18M, N44S, K52N, V57M, and V82I (by IMGT; D356E, L358M, N384S, K392N, V397M, and V422I by EU) in the case of IgG1 antibodies; N44S, K52N, and V82I (IMGT; N384S, K392N, and V422I by EU) in the case of IgG2 antibodies; and Q15R, N44S, K52N, V57M, R69K, E79Q, and V82I (by IMGT; Q355R, N384S, K392N, V397M, R409K, E419Q, and V422I by EU) in the case of IgG4 antibodies.

All possible combinations of the foregoing Fc domain mutations, and other mutations within the antibody variable domains disclosed herein, are contemplated within the scope of the present disclosure.

### Biological Characteristics of the Bispecific Antibodies and Antigen-Binding Molecules

The present disclosure includes antibodies and antigen-binding fragments thereof that bind human 4-1BB and PD-L1 with high affinity. The present disclosure also includes antibodies and antigen-binding fragments thereof that bind human 4-1BB and/or PD-L1 with medium or low affinity, depending on the therapeutic context and particular targeting properties that are desired. For example, in the context of a bispecific antigen-binding molecule, wherein one arm binds 4-1BB and another arm binds a target antigen (e.g., PD-L1), it may be desirable for the target antigen-binding arm to bind the target antigen with high affinity while the anti-4-1BB arm binds 4-1BB with only moderate or low affinity. In this manner, preferential targeting of the antigen-binding molecule to cells expressing the target antigen may be achieved while avoiding general/untargeted 4-1BB binding and the consequent adverse side effects associated therewith.

According to certain embodiments, the present disclosure includes antibodies and antigen-binding fragments of antibodies that bind human 4-1BB (e.g., at 25° C.) with a K_{D} of less than about 200 nM as measured by surface plasmon resonance, e.g., using an assay format as defined in Example 2 herein. In certain embodiments, the antibodies or antigen-binding fragments of the present disclosure bind 4-1BB with a K_{D} of less than about 200 nM, less than about 150 nM, less than about 100 nM, less than about 90 nM, less than about 80 nM, less than about 60 nM, less than about 40 nM, less than about 30 nM, less than 20 nM, less than 10 nM, or less than 5 nM as measured by surface plasmon resonance, e.g., using an assay format as defined in Example 2 herein, or a substantially similar assay. In certain embodiments, the antibodies or antigen-binding fragments of the present disclosure bind 4-1BB with a K_{D} of about 10 nM to about 200 nM.

The present disclosure also includes antibodies and antigen-binding fragments thereof that bind 4-1BB with a dissociative half-life (t½) of greater than about 0.1 minutes as measured by surface plasmon resonance at 25° C. or 37° C., e.g., using an assay format as defined in Example 2 herein, or a substantially similar assay. In certain embodiments, the antibodies or antigen-binding fragments of the present disclosure bind 4-1BB with a t½ of greater than about 0.5 minutes, greater than about 1 minute, greater than about 3 minutes, greater than about 5 minutes, greater than about 10 minutes, greater than about 15 minutes, greater than about 20 minutes, greater than about 30 minutes, greater than about 40 minutes, or greater than about 50 minutes, as measured by surface plasmon resonance at 25° C. or 37° C., e.g., using an assay format as defined in Example 2 herein, or a substantially similar assay.

According to certain embodiments, the present disclosure includes antibodies and antigen-binding fragments of antibodies that bind human PD-L1 (e.g., at 25° C.) with a K_{D} of less than about 1 nM as measured by surface plasmon resonance, e.g., using an assay format as defined in Example 2 herein. In certain embodiments, the antibodies or antigen-binding fragments of the present disclosure bind PD-L1 with a K_{D} of less than about 1 nM, less than about 0.9 nM, less than about 0.8 nM, less than about 0.6 nM, less than about 0.4 nM, less than about 0.3 nM, less than 0.2 nM, less than 0.1 nM, or less than 0.05 nM as measured by surface plasmon resonance, e.g., using an assay format as defined in Example 2 herein, or a substantially similar assay. In certain embodiments, the antibodies or antigen-binding fragments of the present disclosure bind PD-L1 with a K_{D} of about 0.05 nM to about 0.2 nM.

The present disclosure also includes antibodies and antigen-binding fragments thereof that bind PD-L1 with a dissociative half-life (t½) of greater than about 30 minutes as measured by surface plasmon resonance at 25° C. or 37° C., e.g., using an assay format as defined in the Examples herein, or a substantially similar assay. In certain embodiments, the antibodies or antigen-binding fragments of the present disclosure bind PD-L1 with a t½ of greater than about 30 minutes, greater than about 60 minutes, greater than about 120 minutes, greater than about 150 minutes, greater than about 180 minutes, or greater than about 200 minutes, as measured by surface plasmon resonance at 25° C. or 37° C., e.g., using an assay format as defined in Example 2 herein, or a substantially similar assay

The present disclosure includes bispecific antigen-binding molecules (e.g., bispecific antibodies) which are capable of simultaneously binding to human 4-1BB and human PD-L1. According to certain embodiments, the bispecific antigen-binding molecules of the disclosure specifically interact with cells that express 4-1BB and/or PD-L1. The extent to which a bispecific antigen-binding molecule binds cells that express 4-1BB and/or PD-L1 can be assessed by fluorescence activated cell sorting (FACS), as illustrated in Example 3 herein. For example, the present disclosure includes bispecific antigen-binding molecules which specifically bind human cell lines which express 4-1BB but not PD-L1 (e.g., Jurkat cell genetically engineered to express 4-1BB). In some embodiments, the bispecific antigen-binding molecules bind to 4-1BB-expressing human or cynomolgus T-cells with an EC₅₀ value less than 1×10⁻⁵ M. In some embodiments, the bispecific antigen-binding molecules bind to 4-1BB-expressing human or cynomolgus T-cells with an EC₅₀ value of 1×10⁻¹² M to 1×10⁻⁵ M. In certain embodiments, the bispecific antigen-binding molecules bind to 4-1BB-expressing human or cynomolgus T-cells with an EC₅₀ value of 1×10⁻¹² M to 1×10⁻⁹ M. In certain embodiments, the bispecific antigen-binding molecules bind to 4-1BB-expressing human or cynomolgus T-cells with an EC₅₀ value of 1×10⁻¹² M to 1×10⁻¹⁰ M. In certain embodiments, the bispecific antigen-binding molecules bind to the surface of cell lines expressing PD-L1 with an EC₅₀ of less than about 4×10⁻⁹ M. The binding of the bispecific antigen-binding molecules to the surface of cells or cell lines can be measured by an in vitro FACS binding assay as described in Example 3.

The present disclosure includes PD-L1×4-1BB bispecific antigen-binding molecules which are capable of depleting tumor cells in a subject. For example, according to certain embodiments, PD-L1×4-1BB bispecific antigen-binding molecules are provided, wherein a single administration of the antigen-binding molecule to a subject at a therapeutically effective dose causes a reduction in the number of tumor cells in the subject.

The present disclosure includes anti-PD-L1×anti-CD28 bispecific antigen-binding molecules which are capable of activating T cells by engaging PD-L1 on target cells and 4-1BB on T-cells (see Example 3). For example, binding of the anti-PD-L1×anti-4-1BB bispecific antigen-binding molecules to T cells can lead to an increase in IL-2 release (Example 4). As such, the bispecific antigen-binding molecules of the disclosure may prove useful in promoting a T-cell mediated immune response.

The present disclosure includes anti-PD-L1×anti-4-1BB bispecific antigen-binding molecules which are capable of blocking interaction of PD-L1 with PD-1 (see Example 5). As such, the bispecific antigen-binding molecules of the disclosure can be useful for inhibiting the immune checkpoint pathway and reducing T-cell exhaustion, thereby promoting a T-cell mediated immune response.

The present disclosure includes anti-PD-L1×anti-4-1BB bispecific antigen-binding molecules which are capable of binding to PD-L1 expressed on cells surface. A variety of tumor cells express PD-L1, including breast tumor cells (e.g., HeLa, MCF-7 and MDA-MB-231), melanoma cells (e.g., A375), lung tumor cells (e.g., HCC44), ovarian cancer cells (e.g., ES-2, SNU-8, MCAS), pancreatic cancer cells (eig., SNU-324), prostate cancer cells (e.g., DU145). As such, the bispecific antibodies of the disclosure may prove useful in treating a multitude of cancer indications.

The present disclosure includes anti-PD-L1×anti-4-1BB bispecific antigen-binding molecules which are capable of enhancing the cytotoxic potency of anti-tumor associated antigen (TAA)×anti-CD3 bispecific antibodies across a variety of cell lines (See Example 6). In certain embodiments, the TAA is selected from the group consisting of AFP, ALK, BAGE proteins, BCMA, BIRC5 (survivin), BIRC7, β-catenin, brc-abl, BRCA1, BORIS, CA9, carbonic anhydrase IX, caspase-8, CALR, CCR5, CD19, CD20 (MS4A1), CD22, CD30, CD40, CDK4, CEA, CTLA4, cyclin-B1, CYP1B1, EGFR, EGFRvIII, ErbB2/Her2, ErbB3, ErbB4, ETV6-AML, EpCAM, EphA2, Fra-1, FOLR1, GAGE proteins (e.g., GAGE-1, -2), GD2, GD3, GloboH, glypican-3, GM3, gp100, Her2, HLA/B-raf, HLA/k-ras, HLA/MAGE-A3, hTERT, LMP2, MAGE proteins (e.g., MAGE-1, -2, -3, -4, -6, and -12), MART-1, mesothelin, ML-IAP, Muc1, Muc2, Muc3, Muc4, Muc5, Muc16 (CA-125), MUM1, NA17, NY-BR1, NY-BR62, NY-BR85, NY-ESO1, OX40, p15, p53, PAP, PAX3, PAX5, PCTA-1, PLAC1, PRLR, PRAME, PSMA (FOLH1), RAGE proteins, Ras, RGS5, Rho, SART-1, SART-3, STEAP1, STEAP2, TAG-72, TGF-β, TMPRSS2, Thompson-nouvelle antigen (Tn), TRP-1, TRP-2, tyrosinase, and uroplakin-3. The anti-PD-L1×anti-4-1BB antibodies may also prove useful when combined with a checkpoint inhibitor, for example, an antibody to PD-1, or any other checkpoint inhibitor.

### Epitope Mapping and Related Technologies

The epitope on 4-1BB or PD-L1 to which the antigen-binding molecules of the present disclosure bind may consist of a single contiguous sequence of 3 or more (e.g., 3, 4, 5, 6, 7, 8, 9, 10, 11, 12, 13, 14, 15, 16, 17, 18, 19, 20 or more) amino acids of a 4-1BB protein or a PD-L1 protein. Alternatively, the epitope may consist of a plurality of non-contiguous amino acids (or amino acid sequences) of 4-1BB or PD-L1. The antibodies of the disclosure may interact with amino acids contained within a 4-1BB monomer, or may interact with amino acids on two different 4-1BB chains of a 4-1BB dimer. The term “epitope,” as used herein, refers to an antigenic determinant that interacts with a specific antigen-binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

Various techniques known to persons of ordinary skill in the art can be used to determine whether an antigen-binding domain of an antibody “interacts with one or more amino acids” within a polypeptide or protein. Exemplary techniques that can be used to determine an epitope or binding domain of a particular antibody or antigen-binding domain include, e.g., routine crossblocking assay such as that described in Antibodies, Harlow and Lane (Cold Spring Harbor Press, Cold Spring Harb., NY), point mutagenesis (e.g., alanine scanning mutagenesis, arginine scanning mutagenesis, etc.), peptide blots analysis (Reineke, 2004, Methods Mol Biol 248:443-463), protease protection, and peptide cleavage analysis. In addition, methods such as epitope excision, epitope extraction and chemical modification of antigens can be employed (Tomer, 2000, Protein Science 9:487-496). Another method that can be used to identify the amino acids within a polypeptide with which an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water to allow hydrogen-deuterium exchange to occur at all residues except for the residues protected by the antibody (which remain deuterium-labeled). After dissociation of the antibody, the target protein is subjected to protease cleavage and mass spectrometry analysis, thereby revealing the deuterium-labeled residues which correspond to the specific amino acids with which the antibody interacts. See, e.g., Ehring (1999) Analytical Biochemistry 267 (2): 252-259; Engen and Smith (2001) Anal. Chem. 73: 256A-265A. Alternatively, in certain embodiments, the protein of interest binds to the antibody, followed by hydrogen-deuterium exchange. After dissociation of the antibody, the target protein is subjected to protease cleavage and mass spectrometry analysis, thereby revealing the non-deuterium-labeled residues which correspond to the specific amino acids with which the antibody interacts. X-ray crystal structure analysis can also be used to identify the amino acids within a polypeptide with which an antibody interacts.

The present disclosure further includes anti-4-1BB and anti-PD-L1 antibodies that bind to the same epitope as any of the specific exemplary antibodies described herein (e.g., antibodies comprising any of the amino acid sequences as set forth in Tables 1, 3, 6, and 9). Likewise, the present disclosure also includes anti-4-1BB and/or anti-PD-L1 antibodies that compete for binding to 4-1BB and/or PD-L1 with any of the specific exemplary antibodies described herein (e.g., antibodies comprising any of the amino acid sequences as set forth in Tables 1, 3, 6, and 9 herein).

The present disclosure also includes bispecific antigen-binding molecules comprising a first antigen-binding domain that specifically binds human 4-1BB, and a second antigen-binding fragment that specifically binds human PD-L1, wherein the first antigen-binding domain binds to the same epitope on 4-1BB as any of the specific exemplary 4-1BB-specific antigen-binding domains described herein, and/or wherein the second antigen-binding domain binds to the same epitope on PD-L1 as any of the specific exemplary PD-L1-specific antigen-binding domains described herein.

Likewise, the present disclosure also includes bispecific antigen-binding molecules comprising a first antigen-binding domain that specifically binds human 4-1BB, and a second antigen-binding fragment that specifically binds human PD-L1, wherein the first antigen-binding domain competes for binding to 4-1BB with any of the specific exemplary 4-1BB-specific antigen-binding domains described herein, and/or wherein the second antigen-binding domain competes for binding to PD-L1 with any of the specific exemplary PD-L1-specific antigen-binding domains described herein.

One can easily determine whether a particular antigen-binding molecule (e.g., antibody) or antigen-binding domain thereof binds to the same epitope as, or competes for binding with, a reference antigen-binding molecule of the present disclosure by using routine methods known in the art. For example, to determine if a test antibody binds to the same epitope on 4-1BB (or PD-L1) as a reference bispecific antigen-binding molecule of the present disclosure, the reference bispecific molecule is first allowed to bind to a 4-1BB protein (or PD-L1 protein). Next, the ability of a test antibody to bind to the 4-1BB (or PD-L1) molecule is assessed. If the test antibody is able to bind to 4-1BB (or PD-L1) following saturation binding with the reference bispecific antigen-binding molecule, it can be concluded that the test antibody does not compete for binding to 4-1BB (or PD-L1) with the reference bispecific antigen-binding molecule and/or that there is steric interference between antibodies that are binding different sites on the antigen. On the other hand, if the test antibody is not able to bind to the 4-1BB (or PD-L1) molecule following saturation binding with the reference bispecific antigen-binding molecule, then the test antibody competes for binding to 4-1BB (or PD-L1) with the reference bispecific antigen-binding molecule of the disclosure. Additional routine experimentation (e.g., peptide mutation and binding analyses) can then be carried out to confirm whether the observed lack of binding of the test antibody is in fact due to binding to the same epitope as the reference bispecific antigen-binding molecule or if steric blocking (or another phenomenon) is responsible for the lack of observed binding. Experiments of this sort can be performed using ELISA, RIA, Biacore, flow cytometry or any other quantitative or qualitative antibody-binding assay available in the art. In accordance with certain embodiments of the present disclosure, two antigen-binding proteins compete for binding to an antigen if, e.g., a 1-, 5-, 10-, 20- or 100-fold excess of one antigen-binding protein inhibits binding of the other by at least 50% but preferably 75%, 90% or even 99% as measured in a competitive binding assay (see, e.g., Junghans et al., Cancer Res. 1990:50:1495-1502). Alternatively, two antigen-binding proteins may bind to the same epitope if essentially all amino acid mutations in the antigen that reduce or eliminate binding of one antigen-binding protein reduce or eliminate binding of the other. Two antigen-binding proteins may have “overlapping epitopes” if only a subset of the amino acid mutations that reduce or eliminate binding of one antigen-binding protein reduce or eliminate binding of the other.

To determine if an antibody or antigen-binding domain thereof competes for binding with a reference antigen-binding molecule, the above-described binding methodology is performed in two orientations: In a first orientation, the reference antigen-binding molecule is allowed to bind to a 4-1BB protein (or PD-L1 protein) under saturating conditions followed by assessment of binding of the test antibody to the 4-1BB (or PD-L1) molecule. In a second orientation, the test antibody is allowed to bind to a 4-1BB (or PD-L1) molecule under saturating conditions followed by assessment of binding of the reference antigen-binding molecule to the 4-1BB (or PD-L1) molecule. If, in both orientations, only the first (saturating) antigen-binding molecule is capable of binding to the 4-1BB (or PD-L1) molecule, then it is concluded that the test antibody and the reference antigen-binding molecule compete for binding to 4-1BB (or PD-L1). As will be appreciated by a person of ordinary skill in the art, an antibody that competes for binding with a reference antigen-binding molecule may not necessarily bind to the same epitope as the reference antibody, but may sterically block binding of the reference antibody by binding an overlapping or adjacent epitope.

### Preparation of Antigen-Binding Domains and Construction of Bispecific Molecules

Antigen-binding domains specific for particular antigens can be prepared by any antibody generating technology known in the art. Once obtained, two different antigen-binding domains, specific for two different antigens (e.g., 4-1BB and PD-L1), can be appropriately arranged relative to one another to produce a bispecific antigen-binding molecule of the present disclosure using routine methods. (A discussion of exemplary bispecific antibody formats that can be used to construct the bispecific antigen-binding molecules of the present disclosure is provided elsewhere herein). In certain embodiments, one or more of the individual components (e.g., heavy and light chains) of the multispecific antigen-binding molecules of the disclosure are derived from chimeric, humanized or fully human antibodies. Methods for making such antibodies are well known in the art. For example, one or more of the heavy and/or light chains of the bispecific antigen-binding molecules of the present disclosure can be prepared using VELOCIMMUNE™ technology. Using VELOCIMMUNE™ technology (or any other human antibody generating technology), high affinity chimeric antibodies to a particular antigen (e.g., 4-1BB or PD-L1) are initially isolated having a human variable region and a mouse constant region. The antibodies are characterized and selected for desirable characteristics, including affinity, selectivity, epitope, etc. The mouse constant regions are replaced with a desired human constant region to generate fully human heavy and/or light chains that can be incorporated into the bispecific antigen-binding molecules of the present disclosure.

Genetically engineered animals may be used to make human bispecific antigen-binding molecules. For example, a genetically modified mouse can be used which is incapable of rearranging and expressing an endogenous mouse immunoglobulin light chain variable sequence, wherein the mouse expresses only one or two human light chain variable domains encoded by human immunoglobulin sequences operably linked to the mouse kappa constant gene at the endogenous mouse kappa locus. Such genetically modified mice can be used to produce fully human bispecific antigen-binding molecules comprising two different heavy chains that associate with an identical light chain that comprises a variable domain derived from one of two different human light chain variable region gene segments. (See, e.g., US 2011/0195454 for a detailed discussion of such engineered mice and the use thereof to produce bispecific antigen-binding molecules).

### Bioequivalents

The present disclosure encompasses antigen-binding molecules having amino acid sequences that vary from those of the described antibodies but that retain the ability to bind 4-1BB and PD-L1. Such variant molecules comprise one or more additions, deletions, or substitutions of amino acids when compared to parent sequence, but exhibit biological activity that is essentially equivalent to that of the described antigen-binding molecules. Likewise, the antigen-binding molecules-encoding DNA sequences of the present disclosure encompass sequences that comprise one or more additions, deletions, or substitutions of nucleotides when compared to the disclosed sequence, but that encode an antigen-binding molecule that is essentially bioequivalent to the described antigen-binding molecules of the disclosure. Examples of such variant amino acid and DNA sequences are discussed above.

The present disclosure includes antigen-binding molecules that are bioequivalent to any of the exemplary antigen-binding molecules set forth herein. Two antigen-binding proteins or antibodies are considered bioequivalent if, for example, they are pharmaceutical equivalents or pharmaceutical alternatives whose rate and extent of absorption do not show a significant difference when administered at the same molar dose under similar experimental conditions, either single does or multiple dose. Some antibodies will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antigen-binding proteins are bioequivalent if there are no clinically meaningful differences in their safety, purity, and potency.

In one embodiment, two antigen-binding proteins are bioequivalent if a patient can be switched one or more times between the reference product and the biological product without an expected increase in the risk of adverse effects, including a clinically significant change in immunogenicity, or diminished effectiveness, as compared to continued therapy without such switching.

In one embodiment, two antigen-binding proteins are bioequivalent if they both act by a common mechanism or mechanisms of action for the condition or conditions of use, to the extent that such mechanisms are known.

Bioequivalence may be demonstrated by in vivo and in vitro methods. Bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antibody.

Bioequivalent variants of the exemplary bispecific antigen-binding molecules set forth herein may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other contexts, bioequivalent antibodies may include the exemplary bispecific antigen-binding molecules set forth herein comprising amino acid changes which modify the glycosylation characteristics of the antibodies, e.g., mutations which eliminate or remove glycosylation.

### Species Selectivity and Species Cross-Reactivity

The present disclosure, according to certain embodiments, provides antigen-binding molecules that bind to human 4-1BB but not to 4-1BB from other species. The present disclosure also provides antigen-binding molecules that bind to human PD-L1 but not to PD-L1 from other species. The present disclosure also includes antigen-binding molecules that bind to human 4-1BB and to 4-1BB from one or more non-human species; and/or antigen-binding molecules that bind to human PD-L1 and to PD-L1 from one or more non-human species.

According to certain exemplary embodiments of the disclosure, antigen-binding molecules are provided which bind to human 4-1BB and/or human PD-L1 and may bind or not bind, as the case may be, to one or more of mouse, rat, guinea pig, hamster, gerbil, pig, cat, dog, rabbit, goat, sheep, cow, horse, camel, cynomolgus, marmoset, rhesus or chimpanzee 4-1BB and or PD-L1. For example, in a particular exemplary embodiment of the present disclosure, bispecific antigen-binding molecules are provided comprising a first antigen-binding domain that binds human 4-1BB and cynomolgus 4-1BB, and a second antigen-binding domain that specifically binds human PD-L1.

### Immunoconjugates

The disclosure encompasses PD-L1×4-1BB antigen-binding proteins, e.g., antibodies or antigen-binding fragments, such as REGN6191, conjugated to another moiety, e.g., a therapeutic moiety (an “immunoconjugate”). In an embodiment of the disclosure, PD-L1×4-1BB antigen-binding protein, e.g., antibody or antigen-binding fragment, is conjugated to any of the further therapeutic agents set forth herein. As used herein, the term “immunoconjugate” refers to an antigen-binding protein, e.g., an antibody or antigen-binding fragment, which is chemically or biologically linked to another antigen-binding protein, a drug, a radioactive agent, a reporter moiety, an enzyme, a peptide, a protein or a therapeutic agent.

In certain embodiments, the therapeutic moiety may be a cytotoxin, a chemotherapeutic drug, an immunosuppressant or a radioisotope. Cytotoxic agents include any agent that is detrimental to cells. Examples of suitable cytotoxic agents and chemotherapeutic agents for forming immunoconjugates are known in the art, (see for example, WO 05/103081).

### Therapeutic Uses of the Antigen-Binding Molecules

The bispecific antibodies and antigen-binding molecules of the disclosure (and therapeutic compositions comprising the same) are useful, inter alia, for treating any disease or disorder in which stimulation, activation and/or targeting of an immune response would be beneficial. In particular, the PD-L1×4-1BB bispecific antigen-binding molecules of the present disclosure may be used for the treatment, prevention and/or amelioration of a hyperproliferative disease such as cancer. In certain embodiments, the present disclosure provides methods for treating cancer in a subject, comprising administering a therapeutically effective dose of PD-L1×4-1BB antigen-binding molecule, e.g., REGN6191.

A hyperproliferative disease, for the purposes herein, refers to a disease characterized by abnormal, excessive and/or uncontrolled cell growth, e.g., wherein the cells express PD-L1. For example, hyperproliferative diseases include cancers. Exemplary cancers include, but are not limited to esophageal carcinoma, lung squamous cell carcinoma, lung adenocarcinoma, cervical squamous cell carcinoma, glioma, thyroid cancer, lung cancer (e.g., non-small cell lung cancer), colorectal cancer, colon cancer, bladder cancer, rectal cancer, head and neck cancer, stomach cancer, liver cancer, pancreatic cancer, renal cancer, urothelial cancer, prostate cancer, testis cancer, breast cancer, cervical cancer, endometrial cancer, ovarian cancer, gastroesophageal cancer, (e.g., gastroesophageal adenocarcinoma), and melanoma. Accordingly, the antibodies and the bispecific antigen-binding molecules of the present disclosure can be used in treating a wide range of cancers.

Cancer characterized by solid tumor cells or cancerous blood cells may be an PD-L1-expressing cancer e.g., wherein PD-L1 expression in the cells of the particular subject to be treated has been confirmed, includes esophageal carcinoma, lung squamous cell carcinoma, lung adenocarcinoma, cervical squamous cell carcinoma, endometrial adenocarcinoma, bladder urothelial carcinoma, lung cancer (e.g., non-small cell lung cancer), colorectal cancer, rectal cancer, endometrial cancer, skin cancer (e.g., head & neck squamous cell carcinoma), brain cancer (e.g., glioblastoma multiforme), breast cancer, gastroesophageal cancer, (e.g., gastroesophageal adenocarcinoma), prostate cancer and/or ovarian cancer.

The antigen-binding molecules of the present disclosure may also be used to treat, e.g., primary and/or metastatic tumors arising in the colon, lung, breast, ovary, kidney, and bladder (or from any cancer discussed herein).

The antigen-binding molecules of the present disclosure may be used to residual cancer in a subject. As used herein, the term “residual cancer” means the existence or persistence of one or more cancerous cells in a subject following treatment with an anti-cancer therapy.

As used herein, the term “subject” refers to a mammal (e.g., rat, mouse, cat, dog, cow, sheep, horse, goat, rabbit), preferably a human, for example, in need of prevention and/or treatment of cancer. The subject may have a cancer, be predisposed to developing such a condition, and/or would benefit from administration of a bispecific antibody or antigen-binding fragment thereof of the present disclosure. In one embodiment, the subject may have, or be at risk of developing, a hyperproliferative disease.

Methods for treating or preventing a cancer (e.g., a PD-L1-expressing cancer) in a subject in need of said treatment or prevention by administering a therapeutically effective dose amount PD-L1×4-1BB antigen-binding protein, in association with an additional therapeutic agent are part of the present disclosure. Additional therapeutic agents are disclosed elsewhere herein.

An “effective” or “therapeutically effective” dose of PD-L1×4-1BB antigen-binding protein, e.g., antibody or antigen-binding fragment, for treating or preventing a hyperproliferative disease, such as an PD-L1-expressing cancer, is the amount of the antigen-binding protein sufficient to alleviate one or more signs and/or symptoms of the disease in the treated subject, whether by inducing the regression or elimination of such signs and/or symptoms or by inhibiting the progression of such signs and/or symptoms. In an embodiment of the disclosure, a therapeutically effective dose of PD-L1×4-1BB antigen-binding protein is 0.1-2000 mg. The dose amount may vary depending upon the age and the size of a subject to be administered, target disease, conditions, route of administration, and the like. In certain embodiments, the initial dose may be followed by administration of a second or a plurality of subsequent doses of antigen-binding protein in an amount that can be approximately the same or less or more than that of the initial dose, wherein the subsequent doses may be separated by 1-8 weeks.

The dose of antigen-binding molecule administered to a patient may vary depending upon the age and the size of the patient, target disease, conditions, route of administration, and the like. The preferred dose is typically calculated according to body weight or body surface area. Depending on the severity of the condition, the frequency and the duration of the treatment can be adjusted. Effective dosages and schedules for administering a bispecific antigen-binding molecule may be determined empirically; for example, patient progress can be monitored by periodic assessment, and the dose adjusted accordingly.

### Combination Therapies

The bispecific antigen-binding molecules of the present disclosure may be used in combination with one or more agents, for example, in treating a cancer in a subject. In certain embodiments, the bispecific antigen-binding molecules may be administered in combination with one or more agents, for example, a corticosteroid, to reduce or ameliorate one or more adverse side effects, e.g., cytokine storm. In certain embodiments, the bispecific antigen-binding molecules may be administered in combination with one or more therapeutic agents or therapies for enhanced efficacy in treating cancer. Exemplary additional therapeutic agents or therapies that may be combined with or administered in combination with an antigen-binding molecule of the present disclosure include, e.g., chemotherapy (e.g., anti-cancer chemotherapy, for example, paclitaxel, docetaxel, vincristine, cisplatin, carboplatin or oxaliplatin), radiation therapy, surgery, a checkpoint inhibitor, a PD-1 inhibitor (e.g., an anti-PD-1 antibody such as pembrolizumab, nivolumab, or cemiplimab), a CTLA-4 inhibitor, LAG3 inhibitor, TIM3 inhibitor, a GITR agonist, OX40 agonist, 4-1BB agonist, an oncolytic virus, a cancer vaccine, a CAR-T cell, a nucleic acid therapeutic, a stem cell transplant, a modified IL2, modified IL12, IL15, IL6 inhibitor (e.g., sarilumab or tocilizumab), IL4R inhibitor (e.g., dupilumab), EGFR inhibitor, Ang2 inhibitor, VEGF inhibitor, a corticosteroid, a bispecific antibody or antigen-binding fragment thereof that binds CD3 and a tumor associated antigen (TAA) (e.g., MUC16 (mucin 16), PSMA, STEAP2, or any of the TAA disclosed herein). Exemplary bispecific antibodies comprising an antigen-binding domain that binds CD3 include, but are not limited to those described in, e.g., WO2017/053856A1, WO2014/047231A1, WO2018/067331A1 and WO2018/058001A1. PD-L1 is expressed in a wide range of cancers. Accordingly, the bispecific anti-PD-L1×CD28 antibodies of the present disclosure can be used in combination with a wide range of bispecific antibodies comprising an antigen-binding domain that binds CD3 in treatments of various cancers.

The additional agents may be administered just prior to, concurrent with, or shortly after the administration of an antigen-binding molecule of the present disclosure; (for purposes of the present disclosure, such an administration regimen is considered the administration of an antigen-binding molecule “in combination with” an additional agent or therapeutic agent or therapy).

### Pharmaceutical Formulations and Administration

The present disclosure provides compositions that include PD-L1×4-1BB antigen-binding proteins and one or more ingredients; as well as methods of use thereof and methods of making such compositions. Pharmaceutical formulations (e.g., aqueous pharmaceutical formulations that include water) comprising an PD-L1×4-1BB antigen-binding protein or the present disclosure and a pharmaceutically acceptable carrier or excipient are part of the present disclosure.

The pharmaceutical compositions of the disclosure can be formulated with suitable carriers, excipients, and other agents that provide improved transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™, Life Technologies, Carlsbad, CA), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

To prepare pharmaceutical formulations of the PD-L1×4-1BB antigen-binding proteins, e.g., antibodies and antigen-binding fragments thereof (e.g., REGN6191), the antigen-binding protein is admixed with a pharmaceutically acceptable carrier or excipient. See, e.g., Remington's Pharmaceutical Sciences and U.S. Pharmacopeia: National Formulary, Mack Publishing Company, Easton, Pa. (1984); Hardman, et al. (2001) Goodman and Gilman's The Pharmacological Basis of Therapeutics, McGraw-Hill, NY; Gennaro (2000) Remington: The Science and Practice of Pharmacy, Lippincott, Williams, and Wilkins, NY; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, NY. In an embodiment of the disclosure, the pharmaceutical formulation is sterile. Such compositions are part of the present disclosure.

Pharmaceutical formulations of the present disclosure include an PD-L1×4-1BB antigen-binding protein and a pharmaceutically acceptable carrier including, for example, water, buffering agents, preservatives and/or detergents.

The scope of the present disclosure includes desiccated, e.g., freeze-dried compositions, comprising an PD-L1×4-1BB antigen-binding protein, e.g., antibody or antigen-binding fragment thereof, or a pharmaceutical formulation thereof that includes a pharmaceutically acceptable carrier but substantially lacks water.

Various delivery systems are known and can be used to administer the pharmaceutical composition of the disclosure, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of introduction include, but are not limited to, intradermal, intramuscular, intraperitoneal, intravenous, subcutaneous, intranasal, reactal, intestinal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

As discussed herein, the present disclosure provides a vessel (e.g., a plastic or glass vial) or injection device (e.g., syringe, pre-filled syringe or autoinjector) comprising any of the PD-L1×4-1BB antigen-binding proteins herein, e.g., antibodies or antigen-binding fragments thereof, or a pharmaceutical formulation comprising a pharmaceutically acceptable carrier or excipient thereof.

A pharmaceutical composition of the present disclosure can be delivered subcutaneously or intravenously with a standard needle and syringe. In addition, with respect to subcutaneous delivery, a pen delivery device as known in the art, may be used in delivering a pharmaceutical composition of the present disclosure. Such a pen delivery device can be reusable or disposable.

Numerous reusable and disposable pens and autoinjector delivery devices have applications in the subcutaneous delivery of a pharmaceutical composition of the present disclosure. See e.g., AUTOPEN™ (Owen Mumford, Inc., Woodstock, UK) or the HUMIRA™ Pen (Abbott Labs, Abbott Park, IL)

In certain situations, the pharmaceutical composition can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Florida. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline and other isotonic solutions which may be used in combination with an appropriate solubilizing agent. Injectable oily mediums are also part of the present disclosure. Such oily mediums may be combined with a solubilizing agent.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the aforesaid antibody contained is generally about 0.1 to about 2000 mg per dosage form in a unit dose; especially in the form of injection.

### Diagnostic Uses

The bispecific antibodies of the present disclosure may also be used to detect and/or measure 4-1BB or PD-L1, or 4-1BB-expressing or PD-L1-expressing cells in a sample, e.g., for diagnostic purposes. For example, PD-L1×4-1BB antibody or antigen-binding fragment thereof, may be used to diagnose a condition or disease characterized by aberrant expression (e.g., over-expression, under-expression, lack of expression, etc.) of 4-1BB or PD-L1. Exemplary diagnostic assays for 4-1BB or PD-L1 may comprise, e.g., contacting a sample, obtained from a patient, with an antibody of the disclosure, wherein the antibody is labeled with a detectable label or reporter molecule. Alternatively, an unlabeled antibody can be used in diagnostic applications in combination with a secondary antibody which is itself detectably labeled. The detectable label or reporter molecule can be a radioisotope, such as ³H, ¹⁴C, ³²p, ³⁵S, or ¹²⁵I; a fluorescent or chemiluminescent moiety such as fluorescein isothiocyanate, or rhodamine; or an enzyme such as alkaline phosphatase, betagalactosidase, horseradish peroxidase, or luciferase. Specific exemplary assays that can be used to detect or measure 4-1BB or PD-L1 in a sample include enzyme-linked immunosorbent assay (ELISA), radioimmunoassay (RIA), and fluorescence-activated cell sorting (FACS). Samples that can be used in 4-1BB or PD-L1 diagnostic assays according to the present disclosure include any tissue or fluid sample obtainable from a patient which contains detectable quantities of 4-1BB or PD-L1 protein, or fragments thereof, under normal or pathological conditions. Generally, levels of 4-1BB or PD-L1 in a particular sample obtained from a healthy patient (e.g., a patient not afflicted with a disease or condition associated with abnormal 4-1BB or PD-L1 levels or activity) will be measured to initially establish a baseline, or standard, level of 4-1BB or PD-L1. This baseline level of 4-1BB or PD-L1 can then be compared against the levels of 4-1BB or PD-L1 measured in samples obtained from individuals suspected of having a 4-1BB or PD-L1 related disease or condition.

### EXAMPLES

The following examples are put forth so as to provide those of ordinary skill in the art with a complete disclosure and description of how to make and use the methods and compositions of the disclosure, and are not intended to limit the scope of what the inventors regard as their invention.

### Example 1: Construction of Anti-PD-L1×4-1BB Antibodies

### Generation of Anti-PD-L1 Antibodies

Anti-PD-L1 antibodies were obtained by immunizing a genetically engineered mouse comprising DNA encoding human immunoglobulin heavy and kappa light chain variable regions with a human PD-L1 antigen (e.g., SEQ ID NO: 79).

Following immunization, splenocytes were harvested from each mouse and B-cell sorted (as described in US 2007/0280945) using a human PD-L1 fragment as the sorting agent that binds and identifies reactive antibodies (antigen-positive B-cells).

The antibodies were characterized and selected for desirable characteristics including affinity, selectivity, etc. The antibodies may have a desired constant region, for example, wild-type or modified hIgG1 or hIgG4 constant region. As will be appreciated by a person of skill in the art, an antibody with a particular constant region (e.g., modified hIgG1) may be converted to an antibody with a different constant region (e.g., modified hIgG4). While the constant region may vary according to specific use, high-affinity antigen-binding and target specificity characteristics reside in the variable region.

Table 1 sets forth the amino acid sequence identifiers of the heavy and light chain variable regions and CDRs of selected anti-PD-L1 antibodies of the disclosure. The corresponding nucleic acid sequence identifiers are set forth in Table 2.

TABLE 1
Amino Acid Sequence Identifiers for Selected Parental PD-L1 Monoclonal Antibodies

| Antibody | SEQ ID NOS: | | | | | | | |
|---|---|---|---|---|---|---|---|---|
| Designation | HCVR | HCDR1 | HCDR2 | HCDR3 | LCVR | LCDR1 | LCDR2 | LCDR3 |
| mAb9364 | 2 | 4 | 6 | 8 | 18 | 20 | 22 | 24 |
| mAb9373 | 50 | 52 | 54 | 56 | 66 | 68 | 70 | 72 |

TABLE 2
Nucleic Acid Sequence Identifiers for Selected Parental PD-L1 Monoclonal Antibodies

| Antibody | SEQ ID NOS: | | | | | | | |
|---|---|---|---|---|---|---|---|---|
| Designation | HCVR | HCDR1 | HCDR2 | HCDR3 | LCVR | LCDR1 | LCDR2 | LCDR3 |
| mAb9364 | 1 | 3 | 5 | 7 | 17 | 19 | 21 | 23 |
| mAb9373 | 49 | 51 | 53 | 55 | 65 | 67 | 69 | 71 |

### Generation of Anti-4-1BB Antibodies

Anti-4-1BB antibodies were obtained by immunizing a VELOCIMMUNE® mouse (i.e., an engineered mouse comprising DNA encoding human Immunoglobulin heavy and universal light chain variable regions) with human 4-1BB protein fused to the Fc portion of mouse IgG2a, or with DNA encoding 4-1BB.

The antibody immune response was monitored by a 4-1BB-specific immunoassay. When a desired immune response was achieved, anti-4-1BB antibodies were isolated directly from antigen-positive B cells, as described in U.S. Pat. No. 7,582,298.

The antibodies were characterized and selected for desirable characteristics including affinity, selectivity, etc. The antibodies may have a desired constant region, for example, wild-type or modified hIgG1 or hIgG4 constant region. As will be appreciated by a person of skill in the art, an antibody with a particular constant region (e.g., modified hIgG1) may be converted to an antibody with a different constant region (e.g., modified hIgG4). While the constant region may vary according to specific use, high-affinity antigen-binding and target specificity characteristics reside in the variable region.

Table 3 sets forth the amino acid sequence identifiers of the heavy and light chain variable regions and CDRs of selected anti-4-1BB antibodies of the disclosure. The corresponding nucleic acid sequence identifiers are set forth in Table 4.

TABLE 3
Amino Acid Sequence Identifiers for Selected Parental 4-1BB Monoclonal Antibodies

| Antibody | SEQ ID NOS: | | | | | | | |
|---|---|---|---|---|---|---|---|---|
| Designation | HCVR | HCDR1 | HCDR2 | HCDR3 | LCVR | LCDR1 | LCDR2 | LCDR3 |
| mAb25894 | 10 | 12 | 14 | 16 | 18 | 20 | 22 | 24 |
| mAb25898 | 32 | 34 | 36 | 38 | 18 | 20 | 22 | 24 |
| mAb25907 | 42 | 12 | 44 | 46 | 18 | 20 | 22 | 24 |
| mAb25921 | 58 | 60 | 62 | 64 | 66 | 68 | 70 | 72 |

TABLE 4
Nucleic Acid Sequence Identifiers for Selected Parental 4-1BB Antibodies

| Antibody | SEQ ID NOS: | | | | | | | |
|---|---|---|---|---|---|---|---|---|
| Designation | HCVR | HCDR1 | HCDR2 | HCDR3 | LCVR | LCDR1 | LCDR2 | LCDR3 |
| mAb25894 | 9 | 11 | 13 | 15 | 17 | 19 | 21 | 23 |
| mAb25898 | 31 | 33 | 35 | 37 | 17 | 19 | 21 | 23 |
| mAb25907 | 41 | 11 | 43 | 45 | 17 | 19 | 21 | 23 |
| mAb25921 | 57 | 59 | 61 | 63 | 65 | 67 | 69 | 71 |

Generation of Bispecific Antibodies (bsAbs) that Bind 4-1BB and PD-L1

Bispecific antibodies comprising an anti-PD-L1-specific binding domain and an anti-4-1BB-specific binding domain were constructed using standard methodologies, wherein the anti-PD-L1 antigen-binding domain and the anti-4-1BB antigen-binding domain each comprise different, distinct HCVRs paired with a common LCVR. In some instances the bispecific antibodies were constructed utilizing a heavy chain from an anti-4-1BB antibody, a heavy chain from an anti-PD-L1 antibody and a common light chain. Table 5 summarizes the component parts (parental antibody designation) of selected bispecific PD-L1×4-1BB antibodies. Tables 6 and 7 show the amino acid and nucleic acid identifiers, respectively, of selected bispecific antibodies. Table 8 shows the full-length heavy chain and light chain sequences of the selected bispecific antibodies. Additional bispecific antibodies that bind to PD-L1 and 4-1BB may be prepared using the parental monoclonal antibodies having the designations shown in Table 9.

TABLE 5
Summary of Component Parts of Selected Anti-PD-L1 x
Anti-4-1BB Bispecific Antibodies

| Bispecific | Anti-PD-L1 | Anti-4-1BB | Common |
|---|---|---|---|
| Antibody | Antigen- | Antigen- | Light Chain |
| Designation | Binding Domain | Binding Domain | Variable Region |
| REGN6188 | mAb9364 | mAb25894 | 1-39 |
| REGN6189 | mAb9364 | mAb25898 | 1-39 |
| REGN6190 | mAb9364 | mAb25907 | 1-39 |
| REGN6191 | mAb9373 | mAb25921 | 3-20 |

TABLE 6
Amino Acid Sequence Identifiers of Selected Anti-PD-L1 × Anti-4-1BB Bispecific Antibodies

| | Anti-PD-L1 | | | | Anti-4-1BB | | | | Common | | | |
|---|---|---|---|---|---|---|---|---|---|---|---|---|
| Bispecific | Antigen-Binding Domain | | | | Antigen-Binding Domain | | | | Light Chain Variable Region | | | |
| Antibody | | HCDR | HCDR | HCDR | | HCDR | HCDR | HCDR | | LCDR | LCDR | LCDR |
| Designation | HCVR | 1 | 2 | 3 | HCVR | 1 | 2 | 3 | LCVR | 1 | 2 | 3 |
| REGN6188 | 2 | 4 | 6 | 8 | 10 | 12 | 14 | 16 | 18 | 20 | 22 | 24 |
| REGN6189 | 2 | 4 | 6 | 8 | 32 | 34 | 36 | 38 | 18 | 20 | 22 | 24 |
| REGN6190 | 2 | 4 | 6 | 8 | 42 | 12 | 44 | 46 | 18 | 20 | 22 | 24 |
| REGN6191 | 50 | 52 | 54 | 56 | 58 | 60 | 62 | 64 | 66 | 68 | 70 | 72 |

TABLE 7
Nucleic Acid Sequence Identifiers of Selected Anti-PD-L1 × Anti-4-1BB Bispecific Antibodies

| | | | | | | | | | Common | | | |
|---|---|---|---|---|---|---|---|---|---|---|---|---|
| | Anti-PD-L1 | | | | Anti-4-1BB | | | | Light Chain Variable | | | |
| Bispecific | Antigen-Binding Domain | | | | Antigen-Binding Domain | | | | Region | | | |
| Antibody | | HCDR | HCDR | HCDR | | HCDR | HCDR | HCDR | | LCDR | LCDR | LCDR |
| Designation | HCVR | 1 | 2 | 3 | HCVR | 1 | 2 | 3 | LCVR | 1 | 2 | 3 |
| REGN6188 | 1 | 3 | 5 | 7 | 9 | 11 | 13 | 15 | 17 | 19 | 21 | 23 |
| REGN6189 | 1 | 3 | 5 | 7 | 31 | 33 | 35 | 37 | 17 | 19 | 21 | 23 |
| REGN6190 | 1 | 3 | 5 | 7 | 41 | 11 | 43 | 45 | 17 | 19 | 21 | 23 |
| REGN6191 | 49 | 51 | 53 | 55 | 57 | 59 | 61 | 63 | 65 | 67 | 69 | 71 |

TABLE 8
Amino acid and nucleotide sequences for full length immunoglobulin
chains of bispecific antibodies REGN6188, REGN6189, REGN6190
and REGN6191

| Bispecific | HC | | HC | | LC | |
|---|---|---|---|---|---|---|
| Antibody | (PD-L1) | | (4-1BB) | | (PD-L1 & 4-1BB) | |
| Designation | D | P | D | P | D | P |
| REGN6188 | 25 | 26 | 27 | 28 | 29 | 30 |
| REGN6189 | 25 | 26 | 39 | 40 | 29 | 30 |
| REGN6190 | 25 | 26 | 47 | 48 | 29 | 30 |
| REGN6191 | 73 | 74 | 75 | 76 | 77 | 78 |

D = Nucleotide sequence of DNA encoding indicated sequence
P = amino acid of polypeptide for the indicated sequence
Numbers refer to SEQ ID NOs for the indicated sequence
HC is the full length heavy chain for the indicated antibody
LC is the full length light chain for the indicated antibody

Additional bispecific antibodies comprising one HCVR from a parental PD-L1 antibody and the other HCVR arm from a parental 4-1BB antibody may be made using the techniques described herein. The parental PD-L1 antibodies used to generate these additional anti-PD-L1×anti-4-1BB bispecific antibodies have HCVR sequences described above in Table 1. The 4-1BB parental antibodies used to generate these additional anti-PD-L1×anti-4-1BB bispecific antibodies have the amino acid sequences described above in Table 3. These anti-PD-L1 and anti-4-1BB binding domains (pairings) are shown below in Table 9.

TABLE 9
Summary of Parental Antibody Designations for HCVR Arms of
Additional Anti- PD-L1 x Anti-4-1BB Bispecific Antibodies

| Anti-PD-L1 | Anti-4-1BB |
|---|---|
| Antigen-Binding Domain | Antigen-Binding Domain |
| (Parental Antibody Designation | (Parental Antibody Designation and |
| and Sequences found in Table 1) | Sequences found herein in Table 3) |
| mAb9364 | mAb25894 |
| mAb9364 | mAb25898 |
| mAb9364 | mAb25907 |
| mAb9364 | mAb25921 |
| mAb9373 | mAb25894 |
| mAb9373 | mAb25898 |
| mAb9373 | mAb25907 |
| mAb9373 | mAb25921 |

The bispecific antibodies described in the following examples consist of antigen-binding arms that bind to human h4-1BB protein; and human PD-L1 (see Biacore binding data below). Exemplified bispecific antibodies contain a modified (chimeric) IgG4 Fc domain as set forth in U.S. Pat. No. 9,359,437.

The bispecific antibodies created in accordance with the present Example comprise two separate antigen-binding domains (i.e., binding arms). The first antigen-binding domain comprises a heavy chain variable region derived from an anti-4-1BB antibody (“4-1BB-VH”), and the second antigen-binding domain comprises a heavy chain variable region derived from an anti-PD-L1 antibody (“PD-L1-VH”). Both the anti-PD-L1 and the anti-4-1BB share a common light chain. The 4-1BB-VH/PD-L1-VH pairing creates antigen-binding domains that specifically recognize 4-1BB (e.g., on T cells) and PD-L1 (e.g., on tumor cells or antigen presenting cells).

### Controls Used in the Following Examples

Three control constructs were included in the following experiments for comparative purposes: “Comparator 1,” a human monoclonal antibody against 4-1BB having V_{H}/V_{L} sequences of antibody “PF-05082566” according to US 2012/0237498 (Pfizer); “Comparator 2,” a human monoclonal antibody against 4-1BB having V_{H}/V_{L} sequences of antibody “20H4.9” according to US 2014/0193422 (BMS); and “Comparator 3,” a bispecific PD-L1×4-1BB antibody having V_{H}/V_{L} sequences of antibodies “CD137-009-HC7LC2” and “PD-L1-547” according to WO 2019/025545 (GenMab).

### Example 2: Characterization of Bispecific Antibodies that Bind 4-1BB and PD-L1 by Surface Plasmon Resonance

PD-L1 kinetics: Equilibrium dissociation constants (K_{D} values) for human PD-L1 expressed with a C-terminal myc-myc-hexahistidine tag (hPD-L1.mmH, SEQ ID NO: 79) binding to purified anti-PD-L1×4-1BB antibodies were determined using a real-time surface plasmon resonance (SPR) biosensor technology using a Biacore S200 instrument. The CM5 Biacore sensor surface was derivatized by amine coupling with a monoclonal mouse anti-human Fc antibody. All Biacore binding studies were performed in a buffer composed of 0.01M HEPES pH 7.4, 0.15M NaCl, 3 mM EDTA, 0.05% v/v Surfactant P20 (HBS-EP running buffer). Different concentrations of hPD-L1.mmH prepared in HBS-EP running buffer (ranging from 0.37 nM to 30 nM in 3-fold serial dilutions) were injected over the captured antibodies at a flow rate of 50 μL/minute. Antibody-reagent association was monitored for 5 minutes while dissociation in HBS-EP running buffer was monitored for 10 minutes. At the end of each cycle, the antibody capture surface was regenerated using a 12 sec injection of 20 mM phosphoric acid. All binding kinetics experiments were performed at 25° C.

4-1BB Kinetics: Equilibrium dissociation constants (K_{D} values) for human 4-1BB expressed with a C-terminal myc-myc-hexahistidine tag (h4-1BB.mmH, SEQ ID NO: 80) binding to purified anti-PD-L1×4-1BB antibodies were determined using a real-time surface plasmon resonance biosensor technology using a Biacore T200 instrument. The CM5 Biacore sensor surface was derivatized by amine coupling with a monoclonal mouse anti-human Fc antibody. All Biacore binding studies were performed in a buffer composed of 0.01M HEPES pH 7.4, 0.15M NaCl, 3 mM EDTA, 0.05% v/v Surfactant P20 (HBS-EP running buffer). Different concentrations of h4-1BB.mmH prepared in HBS-EP running buffer (ranging from 30 nM to 270 nM in 3-fold serial dilutions) were injected over the captured antibodies at a flow rate of 50 μL/minute. Antibody-reagent association was monitored for 5 minutes while dissociation in HBS-EP running buffer was monitored for 10 minutes. At the end of each cycle, the antibody capture surface was regenerated using a 10 sec injection of 20 mM phosphoric acid. All binding kinetics experiments were performed at 25° C.

Data analysis: The specific SPR-Biacore sensorgrams were obtained by a double referencing procedure. The double referencing was performed by first subtracting the signal of each injection over a reference surface (anti-hFc or anti-mFc) from the signal over the experimental surface (anti-hFc-captured anti-PD-L1×4-1BB antibodies) thereby removing contributions from refractive index changes. In addition, running buffer injections were performed to allow subtraction of the signal changes resulting from the dissociation of captured antibodies or antigens from the coupled anti-hFc or anti-mFc surface. Kinetic association (kₐ) and dissociation (k_{d}) rate constants were determined by fitting the real-time sensorgrams to a 1:1 binding model using Scrubber v2.0c curve fitting software. Binding dissociation equilibrium constants (K_{D}) and dissociative half-lives (t½) were calculated from the kinetic rate constants as:

$${K}_{D} ( M ) = \frac{kd}{ka} , and {t}_{1 / 2} ( min ) = \frac{ln ( 2 )}{60 * kd}$$

Biacore analysis showed that PD-L1×4-1BB bound hPD-L1 with a K_{D} of ˜4.5E-11 to 1.2E-10 and h4-1BB with a K_{D} of ˜1.7E-07 to a.2E-08 (Tables 10 and 11).

TABLE 10
Kinetic and Equilibrium Binding Parameters of hPD-L1 to
Surface-captured anti-PD-L1 × 4-1BB Antibodies at 25° C.

| | | 30 nM | | | | |
|---|---|---|---|---|---|---|
| | mAb Capture | hPD-L1.mmH | | | | |
| Ab PID | (RU) | Bind (RU) | ka (1/M/s) | kd (1/s) | KD (M) | t½ (min) |
| REGN6188 | 297.0 ± 0.8 | 45.1 | 5.94E+05 | 6.73E−05 | 1.13E−10 | 171.5 |
| REGN6189 | 315.4 ± 1.0 | 50.8 | 5.93E+05 | 7.84E−05 | 1.32E−10 | 147.3 |
| REGN6190 | 301.0 ± 0.5 | 46.5 | 5.96E+05 | 6.99E−05 | 1.17E−10 | 165.1 |
| REGN6191 | 300.3 ± 0.6 | 55.9 | 1.12E+06 | 5.53E−05 | 4.95E−11 | 209.0 |
| mAb9364 | 379.9 ± 0.8 | 112.2 | 5.71E+05 | 7.42E−05 | 1.30E−10 | 155.7 |
| mAb9373 | 296.2 ± 1.8 | 103.1 | 9.77E+05 | 5.58E−05 | 5.71E−11 | 206.9 |
| Comparator 3 | 292.5 ± 1.5 | 68.4 | 1.22E+06 | 3.39E−05 | 2.78E−11 | 340.6 |
| Isotype | 303.7 ± 0.5 | 0.5 | NB | NB | NB | NB |
| Control | | | | | | |

* NB: Non-binding

TABLE 11
Kinetic and Equilibrium Binding Parameters of h4-1BB to Surface-captured
anti-PD-L1 × 4-1BB Antibodies at 25° C.

| | | 270 nM | | | | |
|---|---|---|---|---|---|---|
| | mAb Capture | h4-1BB-mmH | | | | |
| Ab PID | (RU) | Bind (RU) | ka (1/M/s) | kd (1/s) | KD (M) | t½ (min) |
| mAb25894 | 205.0 ± 0.6 | 35.1 | 5.30E+05 | 9.02E−02 | 1.70E−07 | 0.1 |
| mAb25898 | 373.6 ± 1.1 | 63.8 | 2.98E+05 | 2.82E−02 | 9.45E−08 | 0.4 |
| mAb25907 | 315.0 ± 0.9 | 67.5 | 5.72E+05 | 1.96E−02 | 3.42E−08 | 0.6 |
| REGN6188 | 272.5 ± 1.4 | 25.4 | 7.42E+05 | 9.48E−02 | 1.28E−07 | 0.1 |
| REGN6189 | 282.8 ± 0.6 | 25.4 | 5.11E+05 | 3.15E−02 | 6.16E−08 | 0.4 |
| REGN6190 | 283.0 ± 0.5 | 30.7 | 6.92E+05 | 2.10E−02 | 3.03E−08 | 0.5 |
| REGN6191 | 279.0 ± 0.5 | 33.7 | 5.11E+04 | 5.88E−04 | 1.15E−08 | 19.6 |
| mAb9364 | 317.1 ± 0.6 | 2.0 | NB | NB | NB | NB |
| Comparator 3 | 285.7 ± 1.3 | 27.2 | 3.95E+05 | 1.56E−04 | 3.94E−10 | 74.1 |

* NB: Non-binding

### Example 3: Characterization of Bispecific PD-L1×4-1BB Antibodies in Binding Cells

hPD-L1×4-1BB bispecific antibody binding to cells was characterized using flow cytometry. In order to assess specificity of antibody binding to PD-L1 and 41BB, OVCAR3 (PD-L1 low endogenous expression), OVCAR3/hPD-L1 (engineered to over-express human PD-L1) and Jurkat/NFkB-Luc/h41BB (PD-L1 low and engineered to express human 41BB) cell lines were used for binding evaluation. Binding was detected by using a labeled secondary antibody and measuring fluorescence on a flow cytometer. Results are shown in Table 12.

Cell lines: Jurkat/NFkB-Luc/h41BB (ACL12578) were Jurkat cells stably transduced with a nuclear factor kappa-light-chain-enhancer of activated B cells (NF-κB)-luciferase reporter construct and engineered to express human 4-1BB; the cell line was maintained in RPMI-1640+10% FBS+L-Glu/PS+500 ug/mL G418+1 ug/mL Puromycin. NIH: OVCAR-3 (HCT728) is an epithelial cell line, isolated from the malignant ascites of a patient with progressive adenocarcinoma of the ovary. Cells are maintained in RPMI-1640+20% FBS+10 mM HEPES+L-glut+P/S+NaPyr+10 ug/mL Bovine Insulin. OVCAR-3/hPD-L1 (ACL14719) are NIH: OVCAR-3 cells engineered to express human PD-L1 (amino acids M1-T290 of accession number NP_054862.1). Cells are maintained in RPMI-1640+20% FBS+10 mM HEPES+L-glut+P/S+NaPyr+10 ug/mL Bovine Insulin+0.25 ug/mL Puromycin.

Assay set up: OVCAR-3+/−PD-L1 cells were lifted with trypsin, washed and resuspended in stain buffer (2% FBS in PBS). OVCAR-3 and Jurkat/NFkB-Luc/h41BB cells were counted and added to wells at 200,000 cells/well. Cells, in wells of 96-well round bottom plates, were pelleted by centrifugation and resuspended in 150 ul stain buffer (PBS containing 2% FBS). Plates were once again centrifuged and subsequently 100 ul of stain buffer alone or stain buffer containing antibody, titrated from 200 nM to 12 pM in a 9-point 1:4 dose titration were added to cells. Cells and antibodies were incubated for 30 min on ice and then washed in stain buffer. Cells were resuspended in 2 μg/ml allophycocyanin (APC) conjugated goat-anti human secondary antibody. A secondary antibody alone control was included. Cells and secondary were incubated for 30 min on ice and then washed in stain buffer. After a subsequent wash with PBS (containing no FBS), cells were resuspended in viability dye (reconstituted in DMSO according to the manufacturer's protocol and diluted 1:1000 in PBS) and incubated for 20 min on ice. They were then washed with stain buffer and resuspended in 2% PFA for 30 min at 4 C. After washing they were filtered and analyzed by flow cytometry. EC₅₀ values of the antibodies were determined from a 4 parameter logistic equation over a 9-point dose response curve (including secondary only control) using GraphPad Prism software.

Controls: In addition to isotype controls and comparator controls, the following controls were used: Control 1: a bispecific antibody with one arm binding to PD-L1 (derived from parental antibody mAb9373) and the other arm binding to an unrelated antigen; Control 2: a bispecific antibody with one arm binding to PD-L1 (derived from parental antibody mAb9364) and the other arm binding to an unrelated antigen.

Binding on Jurkat/NFkB-Luc/h41BB cells: Dose dependent binding of the PD-L1×41BB antibodies (REGN6188, REGN6189, REGN6190, REGN6191) was observed in the presence of Jurkat/NFkB-Luc/h41BB cells, with REGN6191 exhibiting the most potent and highest maximum binding. In comparison, the 41BB bivalent parentals for REGN6188, REGN6189, and REGN6190, (mAb25894, mAb25898, mAb25907, respectively) showed greater maximum binding, while the bivalent parental for REGN6191 (mAb25921), exhibited similar maximum binding, albeit more potent. Comparator 2 displayed dose dependent binding on Jurkat/NFkB-Luc/h41BB cells, with potency and maximum binding in the range of other 41BB bivalent antibodies. Additionally, bispecific controls or bivalent PD-L1, (Control 1, Control 2 and mAb9373, mAb9364, respectively), showed low maximum, however dose dependent, binding on Jurkat/NFkB-Luc/h41BB cells, indicating potential low endogenous expression of PD-L1 on these cells. Relevant isotype controls did not bind Jurkat/NFkB-Luc/h41BB cells.

Binding on OVCAR3 and OVCAR3/PD-L1 cells: Low, dose dependent binding of PD-L1×41BB, PD-L1 bivalent or bispecific controls, was observed on OVCAR3 cells, suggesting a low level of endogenous PD-L1 expression. To note max binding was not much higher than what was observed for isotype controls. On the contrary, for OVCAR3/hPD-L1 cells, both PD-L1×41BB and PD-L1 control antibodies, displayed high maximum, dose dependent binding in the low to sub-nanomolar range, compared to isotype controls.

TABLE 12
Maximum binding and EC50 values of binding for antibodies

| | Jurkat/NFkB-Luc/h41BB | | | OVCAR3 | | | OVCAR3/PD-L1 | | |
|---|---|---|---|---|---|---|---|---|---|
| | Max | Fold | | Max | Fold | | Max | Fold | |
| Antibody | [gMFI] | change | EC₅₀ [M] | [gMFI] | change | EC₅₀ [M] | [gMFI] | change | EC₅₀ [M] |
| R6188 | 5.2E+04 | 22.9 | NC | 1.1E+04 | 4.6 | NC | 9.6E+06 | 2530.1 | 1.9E−09 |
| R6189 | 5.4E+04 | 23.8 | NC | 8.9E+03 | 3.8 | 3.2E−11 | 9.9E+06 | 2616.8 | 2.4E−09 |
| R6190 | 8.2E+04 | 36.2 | NC | 9.1E+03 | 3.9 | 6.1E−11 | 1.0E+07 | 2720.5 | 1.6E−09 |
| R6191 | 1.8E+05 | 79.0 | 3.18−09 | 8.9E+03 | 3.8 | 5.1E−12 | 1.0E+07 | 2737.4 | 1.4E−09 |
| Control 1 | 2.0E+04 | 9.0 | 4.6E−11 | 9.3E+03 | 4.0 | 5.5E−12 | 1.0E+07 | 2715.9 | 1.2E−09 |
| Control 2 | 1.9E+04 | 8.4 | 4.78−10 | 1.7E+04 | 7.4 | NC | 9.1E+06 | 2414.1 | 3.4E−09 |
| mAb9373 | 2.3E+04 | 10.3 | NC | 1.1E+04 | 4.6 | NC | 8.2E+06 | 2159.7 | 6.4E−10 |
| mAb9364 | 2.2E+04 | 9.5 | NC | 1.0E+04 | 4.4 | 8.4E−12 | 8.3E+06 | 2195.4 | 8.6E−10 |
| Comparator 2 | 1.1E+05 | 48.5 | 1.3E−10 | 1.1E+04 | 4.8 | NC | 1.6E+04 | 4.2 | NC |
| Isotype Control 1 | 4.4E+03 | 2.0 | NC | 8.8E+03 | 3.8 | NC | 6.4E+03 | 1.7 | ND |
| Isotype Control 2 | 2.4E+03 | 1.1 | ND | 4.5E+03 | 1.9 | ND | 5.1E+03 | 1.4 | ND |
| Isotype Control 3 | 2.3E+03 | 1.0 | ND | 4.4E+03 | 1.9 | ND | 5.1E+03 | 1.4 | ND |

| | | | | |
|---|---|---|---|---|
| mAb25894 | 1.0E+05 | 44.7 | NC | N/A |
| mAb25898 | 1.2E+05 | 53.8 | 4.06E−11 | N/A |
| mAb25907 | 1.4E+05 | 60.5 | 5.08E−12 | N/A |
| mAb25921 | 1.7E+05 | 76.9 | 2.87E−10 | N/A |

Mean Fluorescence Intensity (MRI) values were plotted using GraphPad Prism and EC50 values of the antibodies were determined using a four-parameter, variable slope, non-linear regression equation over a 9-point dose-response curve, with a 1:4 titration of antibody ranging from 200 nM
Maximum MFI, is the highest MFI along the dose response curve and the fold binding is the maximum MFI divided by the MFI value from secondary antibody alone.
Abbreviations: ND: Not Determined because no dose dependent response was observed; NC: Not calculated because the data did not fit a 4-parameter logistic equation. N/A: Not available because condition was not tested.

### Example 4: Activation of T Cells by PD-L1×4-1BB Antibodies

Two signals, “signal 1” & “signal 2”, are required for proper T cell activation. “Signal 1” is induced by binding of the T cell receptor (TCR) on T cells to peptide-bound major histocompatibility complex (MHC) molecules on antigen presenting cells (APCs). “Signal 2” is provided by engagement of co-stimulatory receptors expressed on T-cells, such as 4-1BB receptor, with ligands expressed on APC's, such as 4-1BBL. Therefore, activation of 4-1BB signaling provides a targeted approach to enhance existing TCR signaling.

PD-L1×4-1BB bispecific antibodies are designed to mimic the natural ligands of 4-1BB, by bridging PD-L1⁺ target cells with 4-1BB⁺ T cells, to provide “signal 2” in order to enhance the activation of T cells in the presence of a “signal 1” provided by a Tumor-associated antigen (TAA)×CD3 bispecific antibody.

### Characterization of T Cell Activation Using a Reporter Assay

The ability of antibodies targeting PD-L1 to interact with and activate 4-1BB, a costimulatory receptor found on T-cells that acts as “signal 2” for proper T-cell activation, was measured in an engineered reporter assay. In this assay, Jurkat cells were engineered to express the reporter gene luciferase under the control of the transcription factor NF-kB (NFkB-Luc) along with the costimulatory receptor 4-1BB (Jurkat/NFkB-Luc/h41BB). Target cells are engineered HEK293 cells expressing CD20 and PSMA only, or in combination with PD-L1. Reporter cells are incubated with target cells and bispecific PD-L1×41BB, or control, antibodies. The ability of PD-L1 targeted bispecifics to specifically bind PD-L1+ target cells and subsequently engage and activate 4-1BB on Jurkat/NFkB-Luc/41BB reporter cells was evaluated via a luminescent readout.

Experimental Procedure: One day before the experiment, Jurkat reporter cells were split to 5×10⁵ cells/ml in RPMI+10% FBS+Penicillin/Streptomycin/L-glutamine (P/S/G)+1 μg/ml Puromycin+500 μg/ml G418 growth media. On the day of the experiment Jurkat/NFkB-luc/h4-1BB cells were resuspended in assay media (RPMI supplemented with 10% FBS+P/S/G) and were added to 96-well white plates at a final concentration of 5×10⁴ cells/well. HEK293/hCD20/hPSMA and HEK293/hCD20/hPSMA/hPD-L1 were lifted with trypsin, washed and resuspend in assay media. Cells were added to the wells of 96-well white plates at a final concentration of 1×10⁴ cells/well. Subsequently, PD-L1×4-1BB, 4-1BB bivalent, Comparator 2, PD-L1 bivalent, and isotype control antibodies were titrated 15 pM to 100 nM in a 1:3 dilution, the final point of the 10-point dilution containing no antibody treatment. All titrations were performed in duplicate and added to the appropriate wells. The antibody dilutions were all generated in assay media. Plates were incubated at 37° C. and 5% CO₂ for 5 hours and then ONE-Glo luciferase substrate was added to each well according to manufacturer's instructions. The luciferase activity was recorded as a luminescence signal using the ENVISION plate reader and expressed as relative light units (RLU). The EC₅₀ values were determined by a 4-parameter logistic equation over a 10-point response curve using GraphPad Prism™. Signal recorded for 10th point on the dilution curve (no antibody condition) was plotted at 5 pM. Maximal RLU is given as the mean max response detected within the tested dose range.

Results: In the presence of HEK293/hCD20/hPSMA/hPD-L1 target cells the PD-L1×4-1BB (REGN6188, REGN6189, REGN6190 and REGN6191) molecules as well as Comparator 2 led to a dose dependent increase in NFkB activity with REGN6191 leading to the most potent response observed. The PD-L1 bivalent, 41BB bivalent, and isotype controls did not increase NFkB activity in a dose dependent manner.

In the absence of PD-L1 expression, Comparator 2 led to a dose dependent increase in NFkB activity. REGN6189 and REGN6190 led to a slight increase in NFkB activity at the highest concentrations tested, however EC50 values could not be generated due to lack of signal plateau. REGN6188 and REGN6191 did not generate dose response curves and maximum responses were comparable to isotype controls.

Luciferase activity and potency values of antibodies are shown in Table 13.

TABLE 13
Maximum luciferase activity and potency values of antibodies

| | HEK293/ | | HEK293/hCD20/ | |
|---|---|---|---|---|
| | hCD20/hPSMA | | hPSMA/hPD-L1 | |
| Antibodies | MAX (RLU) | EC₅₀ [M] | MAX (RLU) | EC₅₀ [M] |
| REGN6188 | 3.59E+04 | NC | 5.62E+05 | 5.58E−10 |
| REGN6189 | 1.75E+05 | NC | 1.18E+06 | 7.03E−09 |
| REGN6190 | 1.20E+05 | NC | 9.73E+05 | 4.48E−09 |
| REGN6191 | 2.00E+04 | ND | 5.79E+05 | 5.49E−11 |
| mAb25894 | 3.43E+04 | 1.48E−08 | 2.98E+04 | NC |
| mAb25898 | 1.90E+04 | ND | 1.90E+04 | ND |
| mAb26907 | 1.85E+04 | ND | 1.81E+04 | ND |
| mAb25921 | 1.80E+04 | ND | 1.80E+04 | ND |
| Comparator 2 | 5.49E+05 | 1.27E−10 | 4.84E+05 | 1.25E−10 |
| mAb9364 | 6.09E+04 | NC | 7.09E+04 | NC |
| mAb9373 | 1.85E+04 | ND | 1.85E+04 | ND |
| Isotypr Control 1 | 2.04E+04 | ND | 2.00E+04 | ND |
| Isotype Control 2 | 1.88E+04 | ND | 1.64E+04 | ND |

Abbreviations:
ND: Not Determined because no dose dependent response was observed;
NC: Not calculated because the data did not fit a 4-parameter logistic equation.

### Characterization of T Cell Activation Using Human T Cells

The ability of PD-L1×4-1BB bispecific antibodies to activate human primary T-cells by engaging PD-L1 on APC's and 4-1BB on T-cells to deliver “signal 2”, as determined by IL2 release, was evaluated in the presence of a human embryonic kidney cancer cell line engineered to express hCD20, hPSMA, and hPD-L1 (HEK293/hCD20/hPSMA/hPD-L1). In this system a bispecific CD20×CD3 antibody is used to provide “signal 1.” HEK293 cells expressing only hCD20 and hPSMA were included as a control to measure activity that may occur in the absence of PD-L1 on APC's

Experimental Procedures: Human peripheral blood mononuclear cells (PBMCs) were isolated from a healthy donor leukocyte pack from Precision for Medicine (Donor 555075) using the following protocol: add 15 mL of density gradient medium (FicollPaque Plus) to an empty 50 ml tube. Dilute leukocyte pack 1:1 with PBS+2% FBS. Layer leukocyte pack on top of density gradient medium. Centrifuge at room temperature at 400G for 30 minutes with brake off. Remove and discard upper plasma layer. Remove and retain the mononuclear cell layer at the plasma-density gradient medium interface. Add PBS+2% FBS to the collected mononuclear cell layer and centrifuge at 300G for 8 minutes. Discard the supernatant and resuspend the resultant PBMCs in appropriate medium. Isolate CD3⁺ T-cells using CD3⁺ T-cell isolation kit (Stemcell) and following manufacturer's recommended instructions.

IL2 release assay: Enriched CD3⁺ T-cells, resuspended in stimulation media, were added into 96-well round bottom plates at a concentration of 1×10⁵ cells/well. Growth-arrested HEK293/hCD20/hPSMA or HEK293/hCD20/hPSMA/hPD-L1 were added to CD3⁺ T-cells at a final concentration of 1×10⁴ cells/well. Following addition of cells, a constant of 0.1 nM CD20×CD3 antibody or its matched isotype control was added to wells containing HEK293/hCD20/hPSMA or HEK293/hCD20/hPSMA/hPD-L1. Subsequently, PD-L1×4-1BB, 4-1BB bivalent, Comparator 2, PD-L1 bivalent, and isotype control antibodies were titrated from 15 pM to 100 nM in a 1:3 dilution and added to wells. The final point of the 10-point dilution contained no titrated antibody. Plates were incubated for 72 hours at 37° C., 5% CO₂ and 5 μL total supernatant was removed and used for measuring IL2. The amount of cytokine in assay supernatant was determined using AlphaLisa kits from PerkinElmer following the manufacturer's protocol. The cytokine measurements were acquired on Perkin Elmer's multilabel plate reader Envision and values were reported as pg/mL. All serial dilutions were tested in duplicate. The EC₅₀ values of the antibodies were determined from a four-parameter logistic equation over a 10-point dose-response curve using GraphPad Prism™ software with the 10^{th} point (no antibody) being represented by 5.1 pM. Maximal IL2 is given as the mean max response detected within the tested dose range.

Results: In the presence of target and “signal 1”, provided by CD20×CD3, PD-L1×4-1BB antibody treatment (REGN6188, REGN6189, REGN6190, REGN6191) as well as Comparator 2 led to a dose dependent increase in IL-2 release, compared to 41BB bivalent, PD-L1 bivalent, and isotype control antibodies, with REGN6191 leading to the greatest maximum increase. In the absence of “signal 1”, none of the antibodies tested enhanced cytokine release from T-cells, while only Comparator 2 led to a dose dependent increase in IL-2 release, in the presence of CD20×CD3 and HEK293/hCD20/hPSMA cells (lacking PD-L1). Luciferase activity and potency values of antibodies are shown in Table 14.

TABLE 14
Maximum IL2 release and potency values of antibodies

| | HEK293/hCD20/ | | | | HEK293/hCD20/ | | | |
|---|---|---|---|---|---|---|---|---|
| | hPSMA + | | HEK293/hCD20/ | | hPSMA/hPD-L1 + | | HEK293/hCD20/ | |
| | CD20 × CD3 | | hPSMA | | CD20 × CD3 | | hPSMA/hPD-L1 | |
| | MAX | EC₅₀ | MAX | EC₅₀ | MAX | EC₅₀ | MAX | EC₅₀ |
| Antibodies | (pg/mL) | [M] | (pg/mL) | [M] | (pg/mL) | [M] | (pg/mL) | [M] |
| REGN6188 | 7.2 | ND | 5.1 | ND | 45.8 | 9.26E−10 | 5.1 | ND |
| REGN6189 | 8.7 | ND | 10.1 | ND | 83.1 | 1.09E−09 | 10.1 | ND |
| REGN6190 | 9.6 | ND | 8.9 | ND | 52.5 | 6.63E−10 | 8.9 | ND |
| REGN6191 | 10.3 | ND | 7.9 | ND | 193.2 | 5.17E−10 | 7.9 | ND |
| mAb25894 | 9.6 | ND | 1.0 | ND | 7.8 | ND | 1.0 | ND |
| mAb25898 | 8.4 | ND | 3.3 | ND | 5.1 | ND | 3.3 | ND |
| mAb26907 | 9.3 | ND | 1.6 | ND | 4.6 | ND | 1.6 | ND |
| mAb25921 | 10.0 | ND | 1.0 | ND | 5.8 | ND | 1.0 | ND |
| Comparator 2 | 96.6 | 4.42E−10 | 7.8 | ND | 33.3 | 8.89E−10 | 7.8 | ND |
| mAb9364 | 13.4 | ND | 3.5 | ND | 9.3 | ND | 3.5 | ND |
| mAb9373 | 11.3 | ND | 2.5 | ND | 10.3 | ND | 2.5 | ND |
| Isotype Control 1 | 15.1 | ND | 1.0 | ND | 9.4 | ND | 1.0 | ND |
| Isotype Control 2 | 10.6 | ND | 12.7 | ND | 12.0 | ND | 12.7 | ND |

Abbreviations: ND: Not Determined because a dose dependent response was not observed; NC: Not calculated because the data did not fit a 4-parameter logistic equation.

### Example 5: Blocking of PD-L1 Binding to PD1 by PD-L1×4-1BB Antibodies

### Characterization of PD-L1 and 4-1BB Blocking by ELISA

ELISA-based blocking assays were developed to determine the ability of PD-L1×4-1BB bispecific antibodies to block the binding of human programmed death-ligand 1 (hPD-L1) to human programmed cell death protein 1 (hPD1) or human tumor necrosis factor receptor superfamily member 9 (h4-1BB) to human tumor necrosis factor ligand superfamily member 9 (h4-1BBL).

The human PD-L1 recombinant protein used in the experiments was comprised of the hPD-L1 extracellular domain (amino acids F19-T239) expressed with the Fc portion of mouse IgG2a at the C-terminus (amino acids E98-K330) (hPD-L1-mFc, accession #NP_054862.1). The human PD1 protein was comprised of the hPD1 extracellular domain (amino acids L25-V170; C93S) expressed with the Fc portion of the human IgG1 at C-terminus (amino acids D104-K330) (hPD1-hFc, accession #NP_005009.2). The human 4-1BB protein was comprised of the h4-1BB extracellular domain (amino acids L24-Q186) expressed with a C-terminal myc-myc-6 histidine tag (h4-1BB-mmh, accession #Q07011-1). The human 4-1BB ligand protein was purchased from AcroBiosystems and was comprised of the h4-1BBL extracellular domain (amino acids R71-E254) expressed with an N-terminal 6 histidine-Flag tag (6His-Flag-h4-1BBL, accession #P41273-1).

In the PD-L1 blocking assay, hPD1-hFc protein (2 g/ml in PBS) was coated onto a 96-well microtiter plate overnight at 4° C. Nonspecific binding sites were subsequently blocked using a 0.5% (w/v) solution of BSA in PBS. In a separate 96-well microtiter plate, a fixed amount of 0.7 nM hPD-L1-mFc was bound for one hour with PD-L1×4-1BB bispecific antibodies, their parental bivalent anti-PD-L1 or anti-4-1BB antibodies, PD-L1×4-1BB comparator or human IgG1, IgG4 or IgG4s isotype antibodies at dilutions ranging from 1.7 pM to 100 nM in PBS+0.5% BSA. The fixed concentration of hPD-L1-mFc was selected to be near the concentration that generated 50% of the maximal binding (EC50 value) of the hPD1plate. The hPD-L1-mFc antibody complexes were then transferred to the hPD1 coated plate. After one hour of incubation at room temperature, the plates were washed, and plate-bound hPD-L1-mFc protein was detected with horseradish peroxidase (HRP) conjugated goat anti-mouse Fcγ fragment specific antibody. The plates were then developed using TMB substrate solution (BD Biosciences) according to the manufacturer's recommended procedure and the absorbance at 450 nm was measured on a SpectraMax I3x plate reader.

The 4-1BB blocking assay was performed similarly to the PD-L1 blocking assay. 6His-Flag-h4-1BBL protein (2 g/ml in PBS) was coated onto a 96-well microtiter plate overnight at 4 □C. In a separate 96-well microtiter plate a fixed amount of 0.25 nM h4-1BB-mmh was bound for one hour with PD-L1×4-1BB bispecific antibodies, parental bivalent anti-PD-L1 or anti-4-1BB antibodies, Comparators 1, 2 or 3, or relevant human isotype control antibodies at dilutions ranging from 1.7 pM to 100 nM in PBS+0.5% BSA. The fixed concentration of h4-1BB-mmh was selected to be near the concentration that generated 50% of the maximal binding (EC50 value) of the 6His-Flag-h4-1BBL plate. The h4-1BB-mmh antibody complexes were then transferred to the 6His-Flag-h4-1BBL coated plate. After one hour of incubation at room temperature, the plates were washed, and plate-bound h4-1BB-mmh protein was detected with HRP conjugated anti-cMyc antibody. The plates were developed using TMB substrate solution (BD Biosciences) and the absorbance at 450 nm was measured on a SpectraMax I3x plate reader.

Binding data were analyzed using a sigmoidal (four-parameter logistic) dose-response model with GraphPad Prism™ software. The calculated IC50 value, defined as the concentration of antibody required to block 50% of hPD-L1-mFc binding to plate-coated hPD1-hFc or h4-1BB-mmh binding to plate-coated 6His-Flag-h4-1BBL, was used as an indicator of blocking potency. The percent blocking of tested antibodies at the highest tested concentration of 100 nM was calculated based on the formula shown below:

$$\% Blocking = 100 - ( \frac{[ Experimental {Signal}_{( 100 nM Ab )} - Background {Signal}_{( buffer )} ]}{[ Maximum {Signal}_{( 0.7 nM hPD - L 1 or 0.25 nM h 4 - 1 BB )} - Background {Signal}_{( buffer )} ]} ) \times 100$$

Antibodies that blocked greater than 50% of binding at the highest concentration tested were classified as blockers and their IC₅₀ values were reported.

Results: For the blocking of PD-L1 interaction with PD1, four PD-L1×4-1BB bispecific antibodies (REGN6188, REGN6189, REGN6190 and REGN6191) displayed concentration dependent blocking of hPD-L1 binding to hPD1 with 97% to 99% blocking at the highest antibody concentration tested (100 nM). The IC50 values for these bispecific antibodies ranged from 0.2 nM to 1.2 nM (Table 15). Their parental bivalent PD-L1 antibodies (mAb9373 and mAb9364) inhibited hPD-L1 binding to hPD1 with a similar percent blocking of 99% and IC50 values of 0.49 nM and 0.52 nM respectively. All parental bivalent 4-1BB antibodies (mAb25894, mAb25898, mAb25907 and mAb25921), the human isotype control antibodies showed no or low blocking of hPD-L1 binding to hPD1, with percentage blocking ranging from—1% to 19%, classifying these antibodies as non-blockers. Comparator 3 inhibited the binding of hD-L1 to hPD1 with 99% blocking and an IC₅₀ of 0.46 nM.

TABLE 15
Summary of PD-L1x4-1BB Bispecific Abs and Their Parental
Abs Blocking Human PD-L1 binding to Human PD1

| | Antibody blocking 0.7 nM hPD- | |
|---|---|---|
| | L1-mFc binding to hPD1-hFc | |
| | | % Blocking at |
| Antibody ID | IC₅₀ (M) | 100 nM mAb |
| REGN6188 | 2.4E−10 | 98 |
| REGN6189 | 4.9E−10 | 99 |
| REGN6190 | 3.1E−10 | 99 |
| REGN6191 | 2.5E−10 | 99 |
| mAb9373 | 4.9E−10 | 99 |
| mAb9364 | 5.2E−10 | 99 |
| mAb25894 | NBL | 15 |
| mAb25898 | NBL | 0 |
| mAb25907 | NBL | −1 |
| mAb25921 | NBL | 2 |
| Isotype control 1 | NBL | 1 |
| Isotype control 2 | NBL | 1 |
| Comparator 3 | 4.6E−10 | 99 |
| Isotype control 3 | NBL | 4 |

NBL: Non-blocking, % blocking is less than or equal to 50%
NA: Not applicable

For the blocking of 4-1BB interaction with 4-1BBL, three out of four evaluated PD-L1×4-1BB bispecific antibodies (REGN6189, REGN6190 and REGN6191) displayed concentration dependent blocking of h4-1BB binding to h4-1BBL with the percent blocking ranging from 64% to 99% at the highest antibody concentration tested and an IC50 value of 1.9 nM for REGN6191. Their parental bivalent 4-1BB antibodies (mAb25898, mAb25907 and mAb25921) displayed similar percent blocking ranging from 70% to 94% and an IC50 value of 0.75 nM for mAb25921, the parental 4-1BB for REGN6191. One out of four PD-L1×4-1BB bispecific antibodies (REGN6188) displayed less than 50% blocking and was classified as a non-blocker, and its parental bivalent 4-1BB antibody (mAb25894) inhibited the binding of h4-1BB to h4-1BBL with 61% blocking. Two comparator antibodies Comparator 1 and Comparator 3 inhibited the binding of h4-1BB to h4-1BBL with 96% and 72% blocking, and IC50 values of 0.33 nM and not determined, respectively. Another comparator antibody Comparator 2 was a non-blocker with 12% blocking. All parental bivalent PD-L1 antibodies (mAb9373 and mAb9364) and the human isotype control antibodies did not display blocking activity, as expected.

TABLE 16
Summary of PD-L1x4-1BB Bispecific Abs and Their Parental Abs
Blocking Human 4-1BB binding to Human 4-1BBL

| | Antibody blocking 0.25 nM h4-1BB- | |
|---|---|---|
| | mmh binding to 6His-Flag-h4- | |
| | 1BBL | |
| | | % Blocking at |
| Antibody ID | IC₅₀ (M) | 100 nM mAb |
| REGN6188 | NBL | 33 |
| REGN6189 | ND | 64 |
| REGN6190 | ND | 85 |
| REGN6191 | 1.9E−09 | 99 |
| mAb9373 | NBL | −6 |
| mAb9364 | NBL | 0 |
| mAb25894 | ND | 61 |
| mAb25898 | ND | 70 |
| mAb25907 | ND | 87 |
| mAb25921 | 7.5E−10 | 94 |
| Isotype control 1 | NBL | −8 |
| Isotype control 2 | NBL | −11 |
| Comparator 1 | ND | 72 |
| Comparator 2 | NBL | 12 |
| Comparator 3 (*) | 3.3E−10 | 96 |
| Isotype control 3 (*) | NBL | −1 |

NBL: Non-blocking, % blocking is less than or equal to 50%
ND: Not determined due to insufficient sigmoidal curvature for IC50 calculation
NA: Not applicable
(*) Comparator 3 and isotype control antibodies were tested in a separate experiment for blocking of h4-1BB binding to h4-1BBL.

### Characterization of PD-L1 Blocking in Cells

Characterization of PD-L1×4-1BB bispecific antibodies in a PD-L1 blocking assay was carried out using WSU-DLCL2, WSU-DLCL2/hPD-L1, and Jurkat/AP1-luc/hPD1 cells. This experiment investigated whether PD-L1 bivalent, PD-L1×4-1BB, bispecific control antibodies or relevant isotype controls, can block PD-L1 from interacting with PD1. CD20×CD3 bispecific antibody provides ‘signal 1’ by engaging CD20 on WSU-DLCL2 target cells (endogenous expression) and CD3 on Jurkat/AP1-luc/hPD1 cells, resulting in an increase in AP-1-Luc activity, observed by an increased luminescent signal. WSU-DLCL2 cells engineered to express PD-L1 will result in decreased AP-1 activity. The ability of PD-L1 antibodies to block PD1 interaction with PD-L1, resulting in recovery of the luciferase signal, was evaluated.

Cell lines: Jurkat/AP1-Luc/hPD1 (ACL8709) was generated from Jurkat cells stably transduced with a human programmed cell death protein 1 construct; the cell line was maintained in RPMI-1640+10% FBS+L-Glu/PS+1 ug/mL Puromycin. WSU-DLCL2/hPD-L1 (ACL 17386) was generated from WSU-DLCL2 cells stably transduced with a human programmed death ligand-1 construct; the cell line was maintained in RPMI-1640+10% FBS+L-Glu/PS+1 ug/mL Puromycin. WSU-DLCL2 cells (HCT883) were maintained in RPMI-1640+10% FBS+L-Glu/PS.

Experimental set-up: One day before the experiment, Jurkat reporter cells were split to 5×10⁵ cells/ml in RPMI+10% FBS+Penicillin/Streptomycin/L-glutamine (P/S/G)+1 μg/ml Puromycin growth media. Jurkat/AP1-Luc/PD1 were resuspended in assay media (RPMI supplemented with 10% FBS+P/S/G) and added to 96-well white plates at a concentration of 5×10⁴ cells/well. Antigen presenting cells with or without PD-L1 expression (WSU-DLCL2/PD-L1 or WSU-DLCL2, respectively) were also resuspended in assay media and added to the plates at a concentration of 2.5×10⁴ cells/well. A bispecific CD20×CD3 antibody was added to all wells at a constant concentration of 1 nM. Subsequently, PD-L1×4-1BB, or relevant monospecific or bispecific controls or isotype control antibodies were titrated 7.6 pM to 500 nM in a 1:4 dilution, the final point of the 10-point dilution containing no titrated antibody (CD20×CD3 constant only). All titrations were performed in duplicate and added to the appropriate wells. The antibody dilutions were all generated in assay media. Plates were incubated at 37° C. and 5% CO₂ for 5 hours and then ONE-Glo luciferase substrate was added to each well according to manufacturer's instructions. The luciferase activity was recorded as a luminescence signal using the ENVISION plate reader and expressed as relative light units (RLU). The EC₅₀ values were determined by a 4-parameter logistic equation over a 10-point response curve using GraphPad Prism™. Signal recorded for 10^{th} point on the dilution curve was plotted at 1.9 pM. Maximal RLU is given as the mean max response detected within the tested dose range. Control 1 and Control 2 (described above) were used in the assay. An anti-PD1 antibody (REGN2810; cemiplimab) was also included in the experiment.

Results: In the presence of CD20×CD3 antibody, WSU-DLCL2 cells expressing PD-L1 exhibited a decrease in AP-1 activity, in comparison to PD-L1 negative WSU-DLCL2 cells.

In the absence of PD-L1 expression on the WSU-DLCL2 cells, PD-L1 bispecific as well as bivalent antibodies and controls did not impact AP-1 activity.

In the presence of PD-L1 expression on the WSU-DLCL2 cells, PD-L1 bispecific, PD-L1 bivalent, REGN2810, and bispecific controls (Control 1 and Control 2) antibodies led to a dose dependent rescue of luminesence signal, which was suppressed by the PD1 and PD-L1 interaction. Isotype controls did not lead to an increase in AP-1 activity.

TABLE 17
Maximum RLU (of AP-1 activity) and Potency values of Antibodies

| | WSU-DLCL2 | | WSU-DLCL2/hPD-L1 | |
|---|---|---|---|---|
| Antibodies | MAX (RLU) | EC50 [M] | MAX (RLU) | EC50 [M] |
| REGN6190 | 1.18E+06 | ND | 1.20E+06 | 4.43E−10 |
| REGN6191 | 1.18E+06 | ND | 1.18E+06 | 1.40E−10 |
| Comparator 3 | 1.23E+06 | ND | 1.11E+06 | 7.87E−11 |
| Control 1 | 1.24E+06 | ND | 1.05E+06 | 2.09E−10 |
| Control 2 | 1.26E+06 | ND | 1.10E+06 | 8.74E−10 |
| mAb9373 | 1.26E+06 | ND | 1.09E+06 | 1.71E−12 |
| mAb9364 | 1.24E+06 | ND | 1.04E+06 | 6.51E−12 |
| REGN2810 | 1.23E+06 | ND | 1.10E+06 | 2.90E−10 |
| Isotype Control 1 | 1.24E+06 | ND | 2.88E+05 | ND |
| Isotype Control 2 | 1.19E+06 | ND | 2.66E+05 | ND |
| Isotype Control 3 | 1.19E+06 | ND | 2.49E+05 | ND |

Abbreviations:
ND: Not Determined because no dose-dependent response was observed;
NC: Not calculated because the data did not fit a 4-parameter logistic equation.

### Example 6: Enhancement of Killing of MUC16+ Cells by Bispecific PD-L1×4-1BB Antibodies in Combination with Muc16×CD3 Antibody

The costimulatory PD-L1×4-1BB bsAbs REGN6190 or REGN6191 bispecific antibodies were tested for their ability to enhance the cytotoxic potency of a TAAxCD3 bsAb REGN4019 targeting MUC16 (WO 2018/067331), a tumor antigen expressed on the surface of OVCAR-3 tumor cells engineered to overexpress hPD-L1. Comparator 3 was included in the experiment.

In order to monitor the killing of MUC16+ cells by flow cytometry in the presence of hPBMC and a combination of a MUC16×CD3 with a PD-L1×4-1BB bispecific antibodies (bsAbs), OVCAR-3/hPD-L1 cells were labeled with 1 uM of the fluorescent tracking dye Violet Cell Tracker. After labeling, cells were plated overnight at 37° C. Separately, human PBMCs were plated in supplemented RPMI media at 1×10⁶ cells/mL and incubated overnight at 37° C. in order to enrich for lymphocytes by depleting adherent macrophages, dendritic cells, and some monocytes. The next day, target cells were co-incubated with adherent cell-depleted naïve PBMC (Effector/Target cell 4:1 ratio), a serial dilution of MUC16×CD3 bispecific antibody REGN4019 or the isotype control (concentration range: 66.7 nM to 150 pM) and a fixed concentration of the PD-L1×4-1BB costimulatory bispecific antibodies at 2.5 ug/ml (16.7 nM) for 72 hours at 37° C. Cells were removed from cell culture plates using Trypsin-EDTA dissociation buffer, and analyzed by Flow cytometry on a BD Celesta cytometer. For Flow cytometry analysis, cells were stained with a dead/live Near IR Reactive (Invitrogen) dye. 2E04 counting beads were added to each well immediately before FACS analysis. 1E04 beads were collected for each sample. For the assessment of specificity of killing, cells were gated on live Violet labeled populations. Percent of live population was recorded and used for the calculation of survival.

T cell activation and upregulation of the PD1 marker were assessed by incubating cells with directly conjugated antibodies to CD2, CD4, CD8, CD25 and PD1, and by reporting the percent of activated (CD25+/CD8+, CD25+/CD4+) T cells and PD1+/CD4+, PD1+/CD8+ T cells out of total T cells (CD2+).

The supernatant of the assay wells from the human PBMC assay were assessed for Th1/Th2 cytokine release using the BD cytometric bead array human kit and following the manufacturer protocol.

Results: In the absence of a targeting ×CD3 bispecific (MUC16×CD3 REGN4019), REGN6190, REGN6191 as well as Comparator 3 were inert. On the other hand, a fixed concentration of both REGN6190 and REGN6191 PD-L1×4-1BB bsAbs successfully enhanced the cytotoxic potency of REGN4019 in the presence of human PBMC against OVCAR-3/hPD-L1 cells, and the cells were killed in a dose-dependent manner. The potency of REGN6190 and REGN6191 was comparable to Comparator 3. Beside the observed target-cell lysis enhancement, T cell activation was increased, with upregulation of CD25 and PD1 expression on CD4+ and CD8+ T cells (Table 18).

TABLE 18
EC50 of killing and T cell activation by bispecific PD-L1 × 4-1BB antibodies in
combination with Muc16 × CD3 antibody (REGN4019)

| | Co- | | T cell activation, EC50 [M] for: | | | |
|---|---|---|---|---|---|---|
| | stimulatory | Cytotoxicity | CD4+/ | CD8+/ | CD4+/ | CD8+/ |
| Signal 1 | signal | EC50 [M] | CD25+ | CD25+ | PD1+ | PD1+ |
| MUC16 × CD3 | — | 6.00E−10 | 1.63E−09 | 1.28E−09 | 7.57E−10 | 1.43E−09 |
| (REGN4019) | | | | | | |
| MUC16 × CD3 | REGN6190 | 2.92E−10 | 4.34E−10 | 3.61E−10 | 1.38E−10 | 6.24E−11 |
| (REGN4019) | | | | | | |
| MUC16 × CD3 | REGN6191 | 2.92E−10 | 4.13E−10 | 2.44E−10 | 1.38E−10 | 5.72E−11 |
| (REGN4019) | | | | | | |
| MUC16 × CD3 | Comparator 3 | 3.25E−10 | 3.50E−10 | 3.25E−10 | 1.50E−10 | 4.51E−11 |
| (REGN4019) | | | | | | |
| control × CD3 | — | N/C | N/C | N/C | N/C | N/C |
| control × CD3 | REGN6190 | N/C | N/C | N/C | N/C | N/C |
| control × CD3 | REGN6191 | N/C | N/C | N/C | N/C | N/C |
| control × CD3 | Comparator 3 | N/C | N/C | N/C | N/C | N/C |

N/C: Not calculable

The supernatants from the cytotoxicity experiment were assessed for cytokine release. REGN4019's cytotoxic potency was associated with accumulation of IFNg in the media, and IFNg media accumulation was enhanced when REGN4019 was combined with PD-L1×4-1BB.

TABLE 19
Cytokine released induced by bispecific PD-L1x4-1BB antibodies in
combination with Muc16xCD3 antibody (REGN4019)

| | | Cytokines accumulation in media | |
|---|---|---|---|
| Signal 1 | Co-stimulatory signal | IFNg, pg/ml | IFNg, EC50 |
| MUC16xCD3 | — | 31 | 4.05E−09 |
| MUC16xCD3 | REGN6190 | 152 | 1.48E−08 |
| MUC16xCD3 | REGN6191 | 128 | 4.47E−09 |
| MUC16xCD3 | Comparator 3 | 138 | 1.89E−09 |
| controlxCD3 | — | 10 | N/C |
| controlxCD3 | REGN6190 | 10 | N/C |
| controlxCD3 | REGN6191 | 10 | N/C |
| controlxCD3 | Comparator 3 | 10 | N/C |

N/C: Not calculable

### Example 7: Robust Anti-Tumor Efficacy of 4 Bispecific PD-L1×4-1BB Antibodies in MC38/hPDL1 Tumor Model

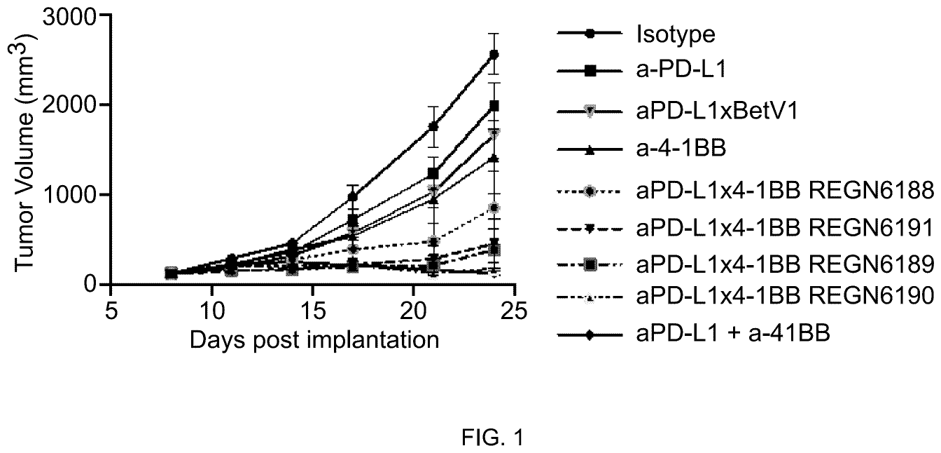

This example relates to an in vivo study demonstrating the efficacy of bispecific PD-L1×4-1BB antibodies in treating human PD-L1-expressing tumors in a murine model of colon cancer in comparison to treatment with anti-PD-L1 or anti-4-1BB antibodies either alone or in combination. Mice were humanized for PD1, PD-L1, and 4-1BB, wherein the mouse genes were knocked out and replaced with their human homologues (hPD1/hPD-L1/h4-1BB mice), and tumors were generated from MC38 colon carcinoma cells that were engineered to knock out mouse PD-L1 and overexpress human PD-L1 (M38-hPDL1-mPDL1KO cells). The following antibodies were used in this study: PD-L1×4-1BB (REGN6188, REGN6189, REGN6190, and REGN6191); PD-L1×BetV1 (a bispecific-control antibody with one arm binding PD-L1 and the other arm binding the unrelated antigen BetV1); anti-PD-L1 (mAb9373, i.e., the parental antibody of REGN6191); anti-4-1BB (mAb25921, i.e., the parental antibody of REGN6191); and Isotype control. In FIG. 1, mAb9373 is referred to as “a-PD-L1”, mAb25921 is referred to as “a-4-1BB”, and PD-L1×BetV1 is referred to as “aPD-L1×BetV1.”

On Day 0, 6 to 7 hPD1/hPD-L1/h4-1BB mice per group were implanted with M38-hPDL1-mPDL1KO cells. Tumors reached an average tumor volume of approximately 100 mm³ on Day 8, and antibodies were administered on Days 8, 11, 15, 19, and 23 at a dose of 5 mg/kg. Tumors were measured approximately twice per week, and tumor-free mice were identified at end of experiment at Day 59.

Results: Administration of any one of REGN6188, REGN6189, REGN6190, or REGN6191 (PD-L1×4-1BB bispecifics) potently controlled tumor growth compared to administration of mAb9373 (a-PD-L1 monospecific), mAb25921 (a-4-1BB monospecific), PD-L1×BetV1 bispecific, or Isotype control (FIG. 1). For example, on Day 24, while the average tumor volume of mice administered Isotype control was approximately 2600 mm³, the average tumor volume of mice administered any one of REGN6188, REGN6189, REGN6190, or REGN6191 was dramatically reduced (e.g., approximately 170 mm³ for REGN6190, FIG. 1). In comparison, also on Day 24, the average tumor volume of mice administered the a-PD-L1 monospecific antibody, mAb9373 was approximately 2000 mm³, the average tumor volume of mice administered PD-L1×BetV1 was approximately 1700 mm³; and the average tumor volume of mice administered the a-4-1BB monospecific antibody, mAb25921 was approximately 1400 mm³ (FIG. 1). Administration of REGN6190 was equally affective at inhibiting tumor growth as administration of the a-4-1BB antibody, mAb9373 and the a-PD-L1 monospecific antibody, mAb25921 in combination. REGN6188, REGN6189, REGN6190, or REGN6191 monotherapy was also superior to any of the mAb9373; PD-L1×BetV1; or mAb25921 monotherapies in inducing complete tumor regression. The fraction of tumor-free mice at end of experiment at Day 59 was between 2/7 and 4/7 for mice administered any one of REGN6188, REGN6189, REGN6190, or REGN6191 (Table 20). In comparison, no mice were tumor free when administered mAb9373 (a-4-1BB), PD-L1×BetV1, mAb25921 (a-PD-L1), or Isotype control at Day 59. The ability of REGN6188, REGN6189, REGN6190, or REGN6191 monotherapy to induce complete tumor regression (2/7 to 4/7 mice tumor free) was equivalent to the ability of mAb9373 (a-PD-L1) and mAb25921 (a-4-1BB) combination therapy to induce complete tumor regression (3/7 mice tumor free) at end of experiment at Day 59. The superior anti-tumor efficacy of PD-L1×4-1BB bispecific antibodies compared to antibodies binding to either PD-L1 or 4-1BB was a surprising result.

TABLE 20
Fraction of mice tumor free at 59 days post-implantation

| Antibody | Fraction tumor free |
|---|---|
| Isotype control | 0/6 |
| mAb9373 | 0/7 |
| PD-L1xBetV1 | 0/7 |
| mAb25921 | 0/7 |
| PD-L1x4-1BB REGN6188 | 2/7 |
| PD-L1x4-1BB REGN6189 | 2/7 |
| PD-L1x4-1BB REGN6190 | 4/7 |
| PD-L1x4-1BB REGN6191 | 2/7 |
| mAb9373 + mAb25921 | 3/7 |

### Example 8: Potent Tumor Growth Control and Survival with Bispecific PD-L1×4-1BB Antibody Monotherapy in MC38/hPDL1 Tumor Model

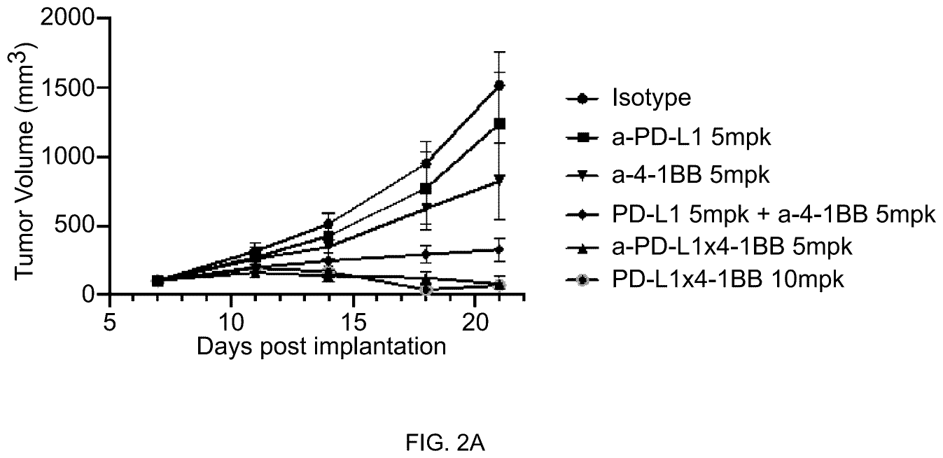

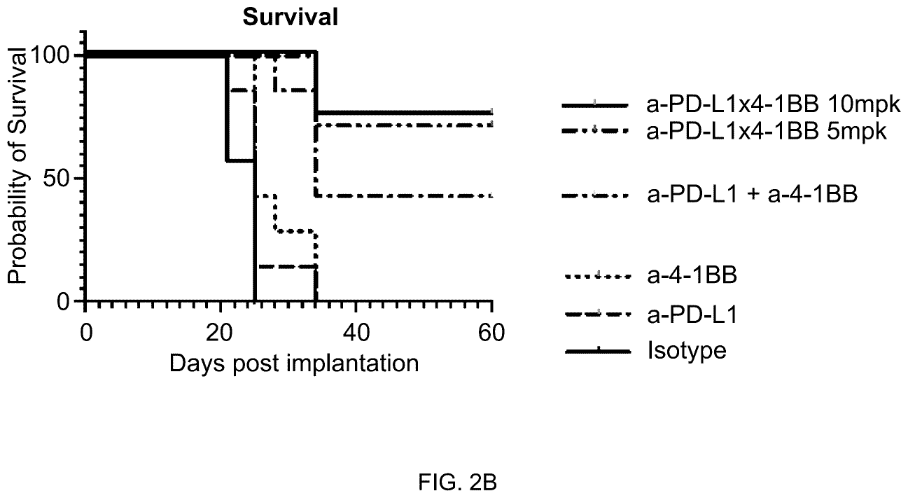

This example relates to an in vivo study demonstrating the potency of a bispecific PD-L1×4-1BB antibody in inhibiting tumor growth and inducing complete and durable regression of human PD-L1-expressing M38 tumors. Mice were humanized for PD1, PD-L1, and 4-1BB, wherein the mouse genes were knocked out and replaced with their human homologues (hPD1/hPD-L1/h4-1BB mice), and tumors were generated from MC38 colon carcinoma cells that were engineered to knock out mouse PD-L1 and overexpress human PD-L1 (M38-hPDL1-mPDL1KO cells). The following antibodies were used in this study: PD-L1×4-1BB (REGN6191); anti-PD-L1, (H4H8314N, US 2022/0184241); anti-4-1BB (mAb25921, i.e., the parental antibody of REGN6191); and Isotype control. In FIGS. 2A-2B, H4H8314N is referred to as “a-PD-L1,” mAb25921 is referred to as “a-4-1BB,” and REGN6191 is referred to as a-PD-L1×4-1BB.

On Day 0, 4 to 7 hPD1/hPD-L1/h4-1BB mice per group were implanted with M38-hPDL1-mPDL1KO cells. Tumors reached an average tumor volume of approximately 100 mm³ on Day 7, and antibodies were administered on Days 7, 11, 14, 18, and 21. All antibodies were administered at a dose of 5 mg/kg, except a second REGN6191 group was dosed at 10 mg/kg. Tumors were measured approximately twice per week until Day 38, and survival was recorded at end of experiment at Day 60.

Results: Administration of REGN6191 potently controlled tumor growth compared to administration of H4H8314N or mAb25921, either alone or in combination (FIG. 2A). For example, at Day 21, while the average tumor volume of mice administered Isotype control was approximately 1500 mm³, the average tumor volume of mice administered REGN6191 was significantly lower (i.e., approximately 80 mm³). In comparison, also on Day 21, the average tumor volume of mice administered H4H8314N was approximately 1200 mm³; the average tumor volume of mice administered mAb25921 was approximately 800 mm³; and the average tumor volume of mice administered H4H8314N and mAb25921 in combination was approximately 300 mm³. Additionally, survival of mice administered REGN6191 was dramatically better than for mice administered H4H8314N or mAb25921 and significantly better than for mice administered H4H8314N and mAb25921 in combination (FIG. 2B). For example, by Day 35, no mice administered H4H8314N or mAb25921 alone had survived, and at end of experiment at Day 60, 43% of mice administered H4H8314N and mAb25921 in combination had survived. In contrast, at Day 60, 73% of mice administered 5 mg/kg REGN6191 had survived and 75% of mice administered 10 mg/kg REGN6191 had survived. REGN6191 monotherapy was also superior to H4H8314N and mAb25921 combination therapy in inducing complete tumor regression by end of experiment at Day 60. The fraction of tumor-free mice was 5/7 mice administered REGN6191 at 5 mg/kg and 3/4 mice administered REGN6191 at 10 mg/kg. In comparison, the fraction of tumor-free mice at end of experiment at Day 60 was 3/7 mice administered H4H8314N and mAb25921 in combination. In summary, administration of PD-L1×4-1BB monotherapy was a surprising improvement over anti-PD-L1 monotherapy, 4-1BB monotherapy, or anti-PD-L1 and 4-1BB combination therapy in inhibiting tumor growth, improving survival, and inducing complete and durable tumor regression.

### Example 9: Superior Tumor Growth Control and Survival of REGN6191 Monotherapy Over a Comparator PD-L1×4-1BB Bispecific Antibody in MC38/hPDL1 Tumor Model

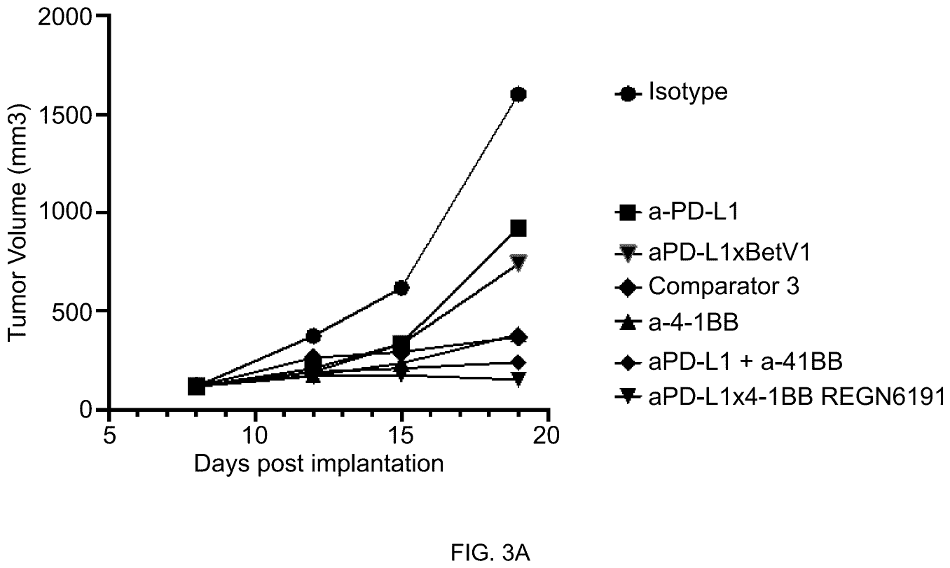

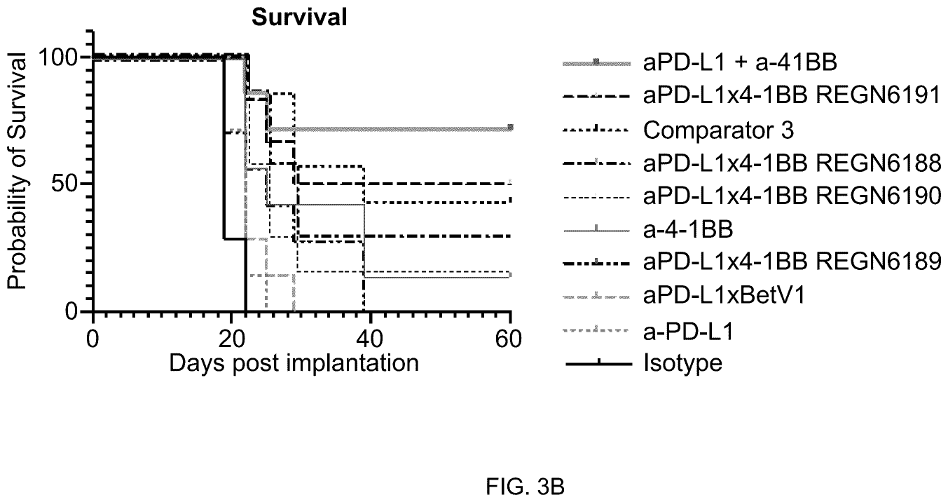

This example relates to an in vivo study demonstrating the superior ability of REGN6191 to inhibit tumor growth and induce complete and durable regression in comparison to a comparator PD-L1×4-1BB antibody. Mice were humanized for PD1, PD-L1, and 4-1BB, wherein the mouse genes were knocked out and replaced with their human homologues (hPD1/hPD-L1/h4-1BB mice), and tumors were generated from MC38 colon carcinoma cells that were engineered to knock out mouse PD-L1 and overexpress human PD-L1 (M38-hPDL1-mPDL1KO cells). The following antibodies were used in this study: 5 PD-L1×4-1BB antibodies (REGN6188, REGN6189, REGN6190, REGN6191, and Comparator 3 (a bispecific PD-L1×4-1BB antibody having V_{H}/V_{L} sequences of antibodies “CD137-009-HC7LC2” and “PD-L1-547” according to WO 2019/025545 (GenMab))); PD-L1×BetV1 (a bispecific-control antibody with one arm binding PD-L1 and the other arm binding the unrelated antigen BetV1); anti-PD-L1 (mAb9373, i.e., the parental antibody of REGN6191); anti-4-1BB (mAb25921, i.e., the parental antibody of REGN619); and Isotype control. In FIGS. 3A-3B mAb9373 is referred to as “a-PD-L1”, mAb25921 is referred to as “a-4-1BB” or “a-41BB”, and PD-L1×BetV1 is referred to as “aPD-L1×BetV1”.

On Day 0, 7 hPD1/hPD-L1/h4-1BB mice per group were implanted with M38-hPDL1-mPDL1KO cells. Tumors reached an average tumor volume of approximately 115 mm³ on Day 8, and antibodies were administered on Days 9, 13, 15 and 19 at a dose of 10 mg/kg. Tumors were measured approximately twice per week until Day 39, and tumor-free mice were identified and survival was recorded at end of experiment at Day 43.

Results: REGN6191 monotherapy controlled tumor growth more effectively than any other therapy, including Comparator 3 monotherapy and mAb9373 and mAb25921 combination therapy (FIG. 3A). For example, at Day 19, while the average tumor volume of mice administered Isotype control was approximately 1600 mm³, the average tumor volume of mice administered REGN6191 was approximately 200 mm³. In comparison, also on Day 19, the average tumor volume of mice administered mAb9373 was approximately 900 mm³; the average tumor volume of mice administered PD-L1×BetV1 was approximately 750 mm³; the average tumor volume of mice administered Comparator 3 or mAb25921 were each approximately 350 mm³; and the average tumor volume of mice administered mAb9373 and mAb25921 in combination was approximately 250 mm³. In addition, survival of mice administered REGN6191 monotherapy was better than for mice administered any of the other monotherapies, including Comparator 3 monotherapy (FIG. 3B). REGN6191 monotherapy was also superior to any of the other monotherapies in inducing complete tumor regression, including Comparator 3 monotherapy (Table 21). While 4/7 mice administered REGN6191 were tumor-free at end of experiment at Day 39, only 3/7 mice were tumor free when administered Comparator 3.

TABLE 21
Fraction of mice tumor free at 60 days post-implantation

| Antibody | Fraction tumor free |
|---|---|
| Isotype control | 0/7 |
| mAb9373 | 0/7 |
| PD-L1xBetV1 | 0/7 |
| mAb25921 | 1/7 |
| REGN6188 | 2/7 |
| REGN6189 | 0/7 |
| REGN6190 | 1/7 |
| REGN6191 | 4/7 |
| Comparator 3 | 3/7 |
| mAb9373 + mAb25921 | 5/7 |

In summary, the anti-tumor effectiveness of REGN6191 was greater than the anti-tumor effectiveness of H4H8314N or mAb25921 either alone or in combination. Also, the ability of REGN6191 monotherapy to inhibit tumor growth, improve survival, and induce complete and durable tumor regression was superior to Comparator 3 monotherapy.

### Example 10: Robust Ability of PD-L1×4-1BB to Enhance the Effectiveness of MUC16×CD3 in Controlling OVCAR-3/hPD-L1 Tumor Growth

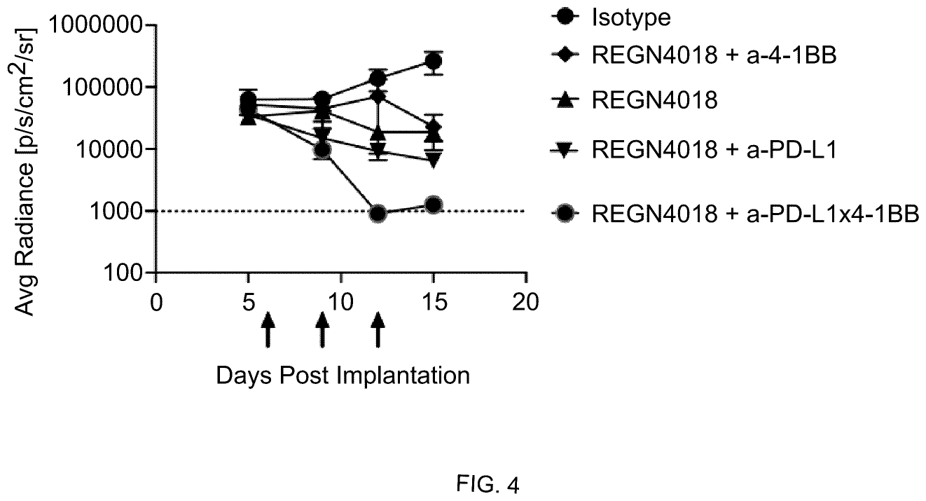

This example relates to an in vivo study demonstrating the ability of a bispecific PD-L1×4-1BB antibody to enhance the efficacy of MUC16×CD3 in treating human PD-L1-overexpressing OVCAR-3 tumors. Mice were the highly immunodeficient SRG mouse strain engrafted with human peripheral blood mononuclear cells (hPBMC) as a source of human immune cells. Tumors were generated from OVCAR-3 tumor cells that endogenously express MUC16 and were engineered to overexpress human PD-L1 and to carry a Luciferase reporter gene (OVCAR-3/Luc/PD-L1 cells). The following antibodies were used in this study: PD-L1×4-1BB (REGN6191); MUC16×CD3 (REGN4018, WO 2018/067331); anti-PD-L1 (H4H8314N, US 2022/0184241); anti-4-1BB (mAb25921, i.e., the parental antibody of REGN6191); and Isotype control. In FIG. 4, H4H8314N is referred to as “a-PD-L1,” mAb25921 is referred to as “a-4-1BB”, and REGN4018 is referred to as “MUC16×CD3.”

Eight days before implantation with tumor cells, NSG mice were engrafted with hPBMC. On Day 0, mice were implanted with OVCAR-3/Luc/PD-L1 cells. On Day 5, bioluminescent imaging (BLI) was performed, and mice were randomized into groups of equal average radiance. Antibodies were administered on Days 6, 9, and 12 at a dose of 5 mg/kg except for REGN4018, which was administered at a suboptimal dose of 0.1 mg/kg. BLI was performed on Days 5, 9, 12, and 15.

Results: REGN6191 greatly improved the ability of REGN4018 to control the growth of OVCAR3/hPD-L1 tumors. In contrast, neither H4H8314N nor mAb25921 significantly improved the anti-tumor activity of REGN4018 (FIG. 4). For instance, at Day 12, while the average radiance of mice administered the combination of REGN4018 and Isotype control was approximately 18,000 p/s/cm²/sr, the average radiance of mice administered the combination of REGN4018 and REGN6191 was greatly reduced (i.e., approximately 900 p/s/cm²/sr). In comparison, also at Day 12, the average radiance of mice administered the combination of REGN4018 and mAb25921 increased (i.e., approximately 70,000 p/s/cm²/sr), and the average radiance of mice administered the combination of REGN4018 and H4H8314N was approximately 9000. The superior anti-tumor efficacy of MUC16×CD3 and PD-L1×4-1BB combination therapy compared to both MUC16×CD3 and anti-PD-L1 combination therapy or MUC16×CD3 and anti-4-1BB combination therapy was a surprising result.

### Example 11: Control of Growth of Tumors not Expressing Human PD-L1 by Combination Treatment with PD-L1×4-1BB and Anti-Human PD-1

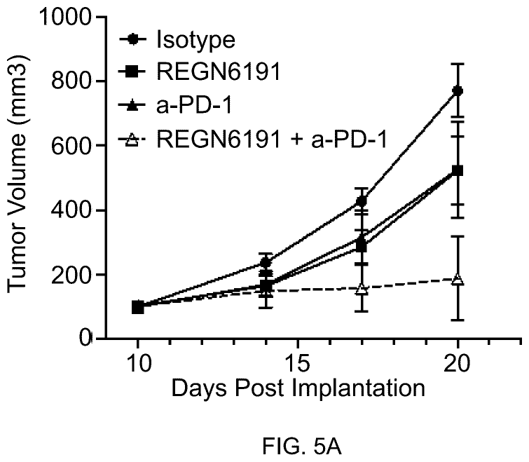

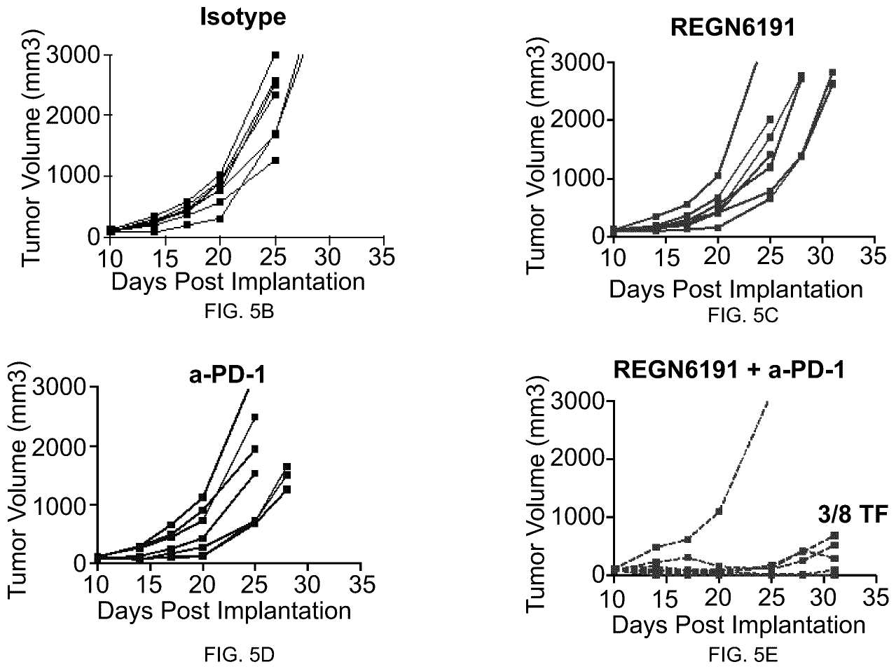

This example relates to an in vivo study demonstrating that the anti-tumor efficacy of a bispecific PD-L1×4-1BB antibody is increased when combined with an anti-human PD-1, even for tumors that do not express human PD-L1. Mice were humanized for PD1, PD-L1, and 4-1BB, wherein the mouse genes were knocked out and replaced with their human homologues (hPD1/hPD-L1/h4-1BB mice). Tumors were generated from parental MC38 cells, which express mouse PD-L1 and not human PD-L1. The following antibodies were used in this study: PD-L1×4-1BB (REGN6191), anti-PD1 (REGN2810; cemiplimab), and Isotype control. In FIGS. 5A-5E, cemiplimab is referred to as “a-PD-1”. Given that (i) the host mice were humanized for PD-L1 and PD1, (ii) the tumor cells did not express human PD-L1, and (iii) REGN6191 does not recognize mouse PD-L1, the ability of cemiplimab to improve the efficacy of REGN6191 indicates a potential role for non-tumor PD-L1+ cells in inhibition of tumor growth.

On Day 0, 7 to 8 hPD1/hPD-L1/h4-1BB mice per group were implanted with MC38 parental tumor cells. Tumors reached an average tumor volume of approximately 100 mm³ on Day 10, and antibodies were administered on Days 10, 14, 17, and 20 at a dose of 10 mg/kg. Tumors were measured approximately twice per week, and tumor-free mice were identified at end of experiment at Day 31.

Results: REGN6191 administered in combination with cemiplimab was stronger at inhibiting the growth of parental MC38 tumors and inducing complete tumor regression than administration of either antibody alone (FIGS. 5A-5E). For example, at Day 20, while the average tumor volume of parental MC38 tumors treated with Isotype control was approximately 800 mm³, the average tumor volume of parental MC38 tumors treated with REGN6191 in combination with cemiplimab was significantly reduced (i.e., approximately 180 mm³, FIG. 5A). In comparison, also at Day 20, the average tumor volume of parental MC38 tumors treated with REGN6191 alone and the average tumor volume of parental MC38 tumors treated with cemiplimab alone were both approximately 500 mm³ (FIG. 5A). In addition, REGN6191 and cemiplimab combination therapy was able to induce complete tumor regression (i.e., 3 out of 8 mice tumor free (TF) at end of experiment at Day 31), while anti-PD1 monotherapy and REGN6191 monotherapy were not (i.e., 0 out of 7 mice tumor free at end of experiment at Day 31). The ability of anti-PD1 and PD-L1×4-1BB combination therapy to robustly inhibit tumor growth and induce complete tumor regression was unexpected, given that parental MC38 tumors do not express human PD-L1. This indicates that non-tumor PD-L1⁺ cells may contribute to the control of tumor growth.

### Example 12: Evaluation of the Safety Profile of Bispecific PD-L1×4-1BB

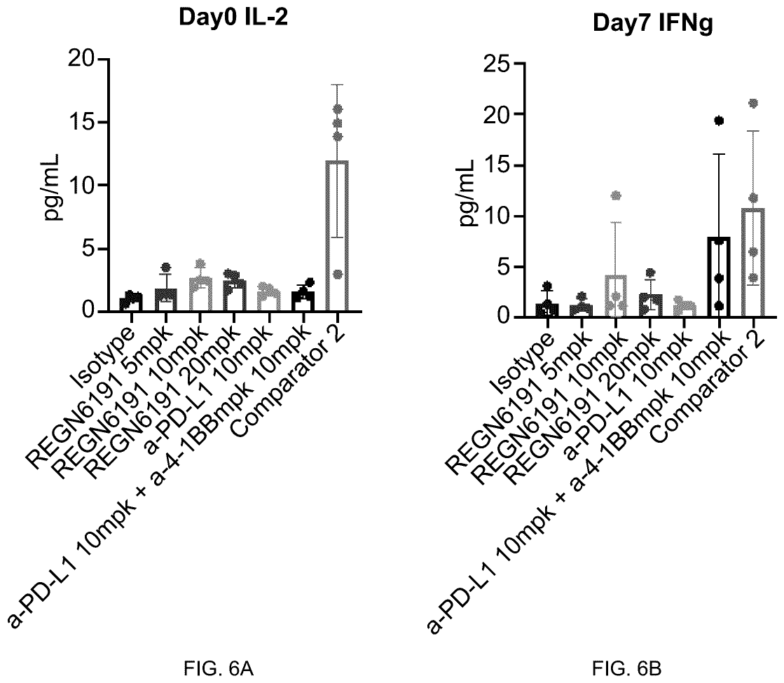

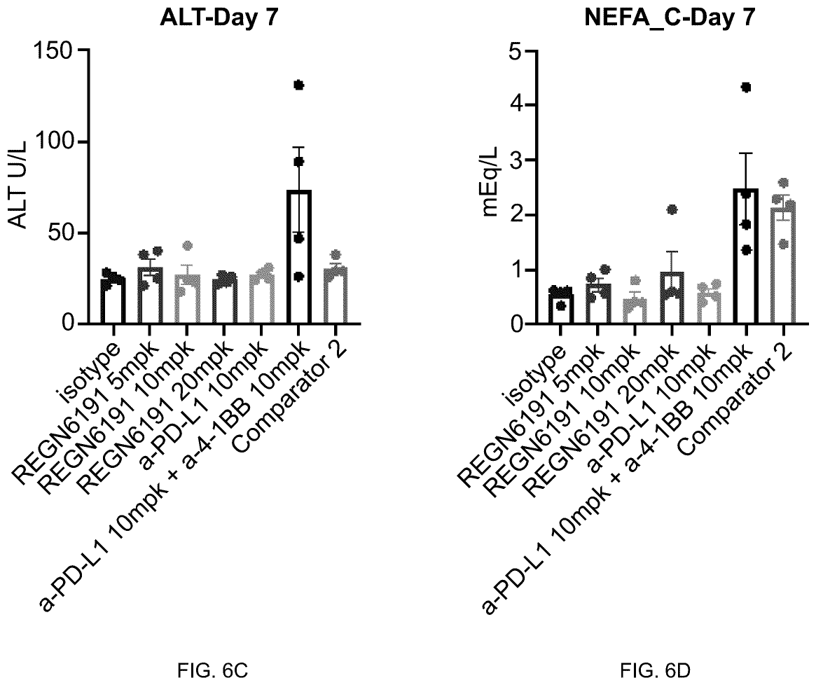

This example relates to an in vivo study demonstrating the advantageous lack of induction of NEFA, ALT, IFNg, and IL-2 by administration of the anti-PD-L1×4-1BB bispecific antibody, REGN6191, compared to administration of Comparator 2 and administration of the combination of anti-PD-L1 and an anti-4-1BB monospecific antibody, wherein the mouse genes were knocked out and replaced with their human homologues (hPD1/hPD-L1/h4-1BB mice). The following antibodies were used in this study: PD-L1×4-1BB (REGN6191), anti-PD-L1 (mAb9373, i.e., the parental antibody of REGN6191); two anti-4-1BB antibodies (mAb25921, i.e., the parental antibody of REGN6191, and Comparator 2), and Isotype control. In FIGS. 6A-6D, mAb9373 is referred to as “a-PD-L1, and mAb25921 is referred to as “a-4-1BB”.

On Days 0, 3, 6, and 7, four hPD1/hPD-L1/h4-1BB mice per group were administered antibodies according to Table 22. Blood was collected on DO, 4 hours after antibody administration and on D7, 24 hours after the third dose. Blood serum was measured for concentration of ALT, AST, LDL, HDL, NEFA, IL-1B, IL-2, IL-5, IL-6, KC/GRO, IL-10, and TNF-a.

TABLE 22
Dose of antibodies used for each group of mice

| Group | Antibodies and doses |
|---|---|
| 1 | 10 mg/kg Isotype control |
| 2 | 5 mg/kg REGN6191 |
| 3 | 10 mg/kg REGN6191 |
| 4 | 20 mg/kg REGN6191 |
| 5 | 10 mg/kg mAb9373 |
| 6 | 10 mg/kg mAb9373 + 10 mg/kg mAb25921 |
| 7 | 10 mg/kg Comparator 2 |

Results: Compared to REGN6191, administration of Urelumab induced significantly greater release of IL-2 on Day 0 and significantly greater release of IFNg and NEFA at Day 7 (FIGS. 6A, 6B, and 6D). Compared to REGN6191, administration of mAb9373 in combination with mAb25921 induced significantly greater release of IFNg, ALT, and NEFA at Day 7 (FIGS. 6B-6D).

### Example 13: Strong Improvement of Anti-Tumor Efficacy of Low-Dose EGFR×CD3 when Combined with PD-L1×4-1BB in A431/PBMC Xenograph Model

This example relates to an in vivo study demonstrating the ability of PD-L1×4-1BB to act synergistically with an anti-tumor associated antigen (TAA)×CD3 bispecific antibody to inhibit tumor growth in the A431/PBMC xenograph model of human skin cancer. The exemplary TAAxCD3 antibody used in this study was EGFR×CD3. Mice were the highly immunodeficient SRG mouse strain engrafted with human peripheral blood mononuclear cells (hPBMC) as a source of human immune cells, and tumors were generated from A431 cells. The following antibodies were used in this study: PD-L1×4-1BB (REGN6191), PD-L1×BetV1 (a bispecific-control antibody with one arm binding PD-L1 and the other arm binding the unrelated antigen BetV1) and an exemplary EGFR×CD3 bispecific antibody.

On Day 0, 5 SRG mice per group were implanted with 2×10⁶ A431 cells and 1×10⁶ hPBMC. Antibodies were administered on Days 0, 3, 7, and 11. All groups were administered EGFR×CD3 at a dose of 0.01 mg/kg. In addition, each group was also administered one of the following: vehicle control, PD-L1×BetV1 at 0.1 mg/kg, or REGN6191 at 0.1 mg/kg. Tumors were measured approximately twice per week, and tumor-free mice were identified at end of experiment at Day 34.

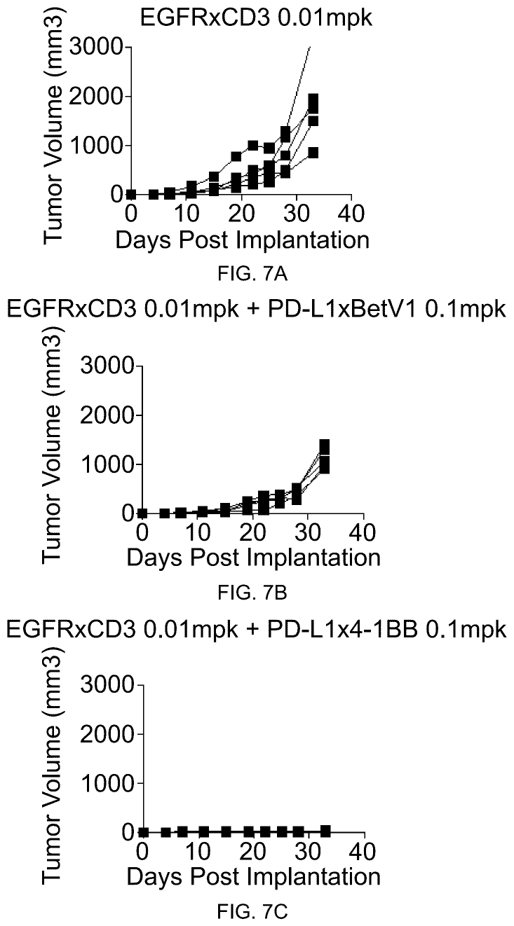

Results: REGN6191 increased the ability of a suboptimal dose of EGFR×CD3 to inhibit tumor growth to a remarkable degree (FIG. 7A-7C). Fox example, at Day 34, while mice administered a suboptimal dose of EGFR×CD3 alone exhibited tumor volumes ranging from 800 mm³ to greater than 3000 mm³, mice administered a suboptimal dose of EGFR×CD3 in combination with REGN6191 exhibited a dramatic improvement in inhibition of tumor growth, i.e., no tumors were detectable at any time point. In contrast, also at Day 34, mice administered a suboptimal dose of EGFR×CD3 in combination with PD-L1×BetV1 exhibited tumor volumes ranging from approximately 800 to 1400 mm³. In summary, the combination of PD-L1×4-1BB and a suboptimal dose of the exemplary TAAxCD3 antibody, EGFR×CD3, was able to profoundly inhibit tumor growth compared to the combination of PD-L1×BetV1 and a suboptimal dose of EGFR×CD3. This was a striking and unexpected result.

The present disclosure is not to be limited in scope by the specific embodiments described herein. Indeed, various modifications of the disclosure in addition to those described herein will become apparent to those skilled in the art from the foregoing description and the accompanying figures. Such modifications are intended to fall within the scope of the appended claims. All patents, applications and non-patent publications mentioned in this specification are incorporated herein by reference in their entireties.

1. An isolated bispecific antigen-binding molecule comprising:
(a) a first antigen-binding domain that specifically binds human 4-1BB, with a K_{D} of less than about 2×10⁻⁷ M as measured by surface plasmon resonance at 25° C.; and
(b) a second antigen-binding domain that specifically binds a human programmed death-ligand 1 (PD-L1) with a K_{D} of less than about 2×10⁻¹⁰M as measured by surface plasmon resonance at 25° C.

2. The isolated bispecific antigen-binding molecule of claim 1, wherein the bispecific antigen-binding molecule binds to the surface of human T cells with an EC₅₀ of less than about 4×10⁻⁹ M as measured by an in vitro FACS binding assay.

3. The isolated bispecific antigen-binding molecule of claim 1, wherein the bispecific antigen-binding molecule binds to the surface of a cell expressing PD-L1 with an EC₅₀ of less than about 4×10⁻⁹ M as measured by an in vitro FACS binding assay.

4. The isolated bispecific antigen-binding molecule of claim 1, wherein the bispecific antigen-binding molecule blocks PD-L1 binding to PD-1 with an IC₅₀ of less than about 1.3 nM as measured by an ELISA-based blocking assay.

5. The isolated bispecific antigen-binding molecule of claim 1, wherein the bispecific antigen-binding molecule, in combination with a bispecific MUC16×CD3 antibody mediates in vitro T cell killing of OVCAR-3 cells expressing PD-L1 with an EC₅₀ of less than about 10⁻¹⁰ M.

6. The isolated bispecific antigen-binding molecule of claim 1, wherein the first antigen-binding domain comprises:
(a) three heavy chain complementarity determining regions (HCDR1, HCDR2 and HCDR3) contained within a heavy chain variable region (HCVR) comprising the amino acid sequence selected from the group consisting of SEQ ID NOs: 58, 42, 32 and 10, or a variant thereof; and
(b) three light chain complementarity determining regions (LCDR1, LCDR2 and LCDR3) contained within a light chain variable region (LCVR) comprising the amino acid sequence selected from the group consisting of SEQ ID NOs: 66 and 18, or a variant thereof.

7. The isolated bispecific antigen-binding molecule of claim 6, comprising a HCDR1 comprising the amino acid sequence selected from the group consisting of SEQ ID NOs: 60, 34 and 12, a HCDR2 comprising the amino acid sequence selected from the group consisting of SEQ ID NOs: 62, 44, 36 and 14, and a HCDR3 comprising the amino acid sequence selected from the group consisting of SEQ ID NOs: 64, 46, 38 and 16.

8. The isolated bispecific antigen-binding molecule of claim 6, comprising a LCDR1 comprising the amino acid sequence selected from the group consisting of SEQ ID NOs: 68 and 20, a LCDR2 comprising the amino acid sequence selected from the group consisting of SEQ ID NOs: 70 and 22, and a LCDR3 comprising the amino acid sequence selected from the group consisting of SEQ ID NO: 72 and 24.

9. The isolated bispecific antigen-binding molecule of claim 7, wherein the first antigen-binding domain comprises:
a HCVR comprising the amino acid sequence of SEQ ID NO: 58, or a variant thereof, and a LCVR comprising the amino acid sequence of SEQ ID NO: 66, or a variant thereof.

10. The isolated bispecific antigen-binding molecule of claim 7, wherein the first antigen-binding domain comprises a HCVR comprising the amino acid sequence of SEQ ID NO: 42, or a variant thereof, and a LCVR comprising the amino acid sequence of SEQ ID NO: 18, or a variant thereof.

11. The isolated bispecific antigen-binding molecule of claim 7, wherein the first antigen-binding domain comprises a HCVR comprising the amino acid sequence of SEQ ID NO: 32, or a variant thereof, and a LCVR comprising the amino acid sequence of SEQ ID NO: 18, or a variant thereof.

12. The isolated bispecific antigen-binding molecule of claim 7, wherein the first antigen-binding domain comprises a HCVR comprising the amino acid sequence of SEQ ID NO: 10, or a variant thereof, and a LCVR comprising the amino acid sequence of SEQ ID NO: 18, or a variant thereof.

13. The isolated bispecific antigen-binding molecule of claim 1, wherein the second antigen-binding domain comprises:
(a) three heavy chain complementarity determining regions (HCDR1, HCDR2 and HCDR3) contained within a heavy chain variable region (HCVR) comprising the amino acid sequence selected from the group consisting of SEQ ID NOs: 50 and 2, or a variant thereof; and
(b) three light chain complementarity determining regions (LCDR1, LCDR2 and LCDR3) contained within a light chain variable region (LCVR) comprising the amino acid sequence selected from the group consisting of SEQ ID NOs: 66 and 18, or a variant thereof.

14. The isolated bispecific antigen-binding molecule of claim 13, wherein the second antigen-binding domain comprises:
(a) a HCDR1 comprising the amino acid sequence of SEQ ID NO: 52 or SEQ ID NO: 4;
(b) a HCDR2 comprising the amino acid sequence of SEQ ID NO: 54 or SEQ ID NO: 6; and
(c) a HCDR3 comprising the amino acid sequence of SEQ ID NO: 58 or SEQ ID NO: 8.

15. The isolated bispecific antigen-binding molecule of claim 13, wherein the second antigen-binding domain comprises a LCDR1 comprising the amino acid sequence selected from the group consisting of SEQ ID NOs: 68 and 20, a LCDR2 comprising the amino acid sequence selected from the group consisting of SEQ ID NOs: 70 and 22, and a LCDR3 comprising the amino acid sequence selected from the group consisting of SEQ ID NO: 72 and 24.

16. The isolated bispecific antigen-binding molecule of claim 15, wherein the second antigen-binding domain comprises:
(a) HCDR1, HCDR2, HCDR3 domains, respectively, comprising the amino acid sequences of SEQ ID NOs: 52, 54, 56; and LCDR1, LCDR2, LCDR3 domains, respectively, comprising the amino acid sequences of SEQ ID NOs: 68, 70, 72; or
(b) HCDR1, HCDR2, HCDR3 domains, respectively, comprising the amino acid sequences of SEQ ID NOs: 4, 6, 8; and LCDR1, LCDR2, LCDR3 domains, respectively, comprising the amino acid sequences of SEQ ID NOs: 20, 22, 24.

17. The isolated bispecific antigen-binding molecule of claim 16, wherein the second antigen-binding domain comprises:
(a) a HCVR comprising the amino acid sequence of SEQ ID NO: 50, or a variant thereof, and a LCVR comprising the amino acid sequence of SEQ ID NO: 66, or a variant thereof; or
(b) a HCVR comprising the amino acid sequence of SEQ ID NO: 2, or a variant thereof, and a LCVR comprising the amino acid sequence of SEQ ID NO: 18, or a variant thereof.

18. An isolated bispecific antigen-binding molecule, comprising:
(a) a first antigen-binding domain that specifically binds human 4-1BB wherein the first antigen-binding domain comprises HCDR1, HCDR2, HCDR3 domains, respectively, comprising the amino acid sequences of SEQ ID NOs: 60, 62, 64, and LCDR1, LCDR2, LCDR3 domains, respectively, comprising the amino acid sequences of SEQ ID NOs: 68, 70, 72; and
(b) a second antigen-binding domain that specifically binds human PD-L1 wherein the second antigen-binding domain comprises HCDR1, HCDR2, HCDR3 domains, respectively, comprising the amino acid sequences of SEQ ID NOs: 52, 54, 56, and LCDR1, LCDR2, LCDR3 domains, respectively, comprising the amino acid sequences of SEQ ID NOs: 68, 70, 72.

19. An isolated bispecific antigen-binding molecule, comprising:
(a) a first antigen-binding domain that specifically binds human 4-1BB wherein the first antigen-binding domain comprises HCDR1, HCDR2, HCDR3 domains, respectively, comprising the amino acid sequences of SEQ ID NOs: 12, 44, 46, and LCDR1, LCDR2, LCDR3 domains, respectively, comprising the amino acid sequences of SEQ ID NOs: 20, 22, 24; and
(b) a second antigen-binding domain that specifically binds human PD-L1 wherein the second antigen-binding domain comprises HCDR1, HCDR2, HCDR3 domains, respectively, comprising the amino acid sequences of SEQ ID NOs: 4, 6, 8, and LCDR1, LCDR2, LCDR3 domains, respectively, comprising the amino acid sequences of SEQ ID NOs: 20, 22, 24.

20. An isolated bispecific antigen-binding molecule, comprising:
(a) a first antigen-binding domain that specifically binds human 4-1BB wherein the first antigen-binding domain comprises HCDR1, HCDR2, HCDR3 domains, respectively, comprising the amino acid sequences of SEQ ID NOs: 34, 36, 38, and LCDR1, LCDR2, LCDR3 domains, respectively, comprising the amino acid sequences of SEQ ID NOs: 20, 22, 24; and
(b) a second antigen-binding domain that specifically binds human PD-L1 wherein the second antigen-binding domain comprises HCDR1, HCDR2, HCDR3 domains, respectively, comprising the amino acid sequences of SEQ ID NOs: 4, 6, 8, and LCDR1, LCDR2, LCDR3 domains, respectively, comprising the amino acid sequences of SEQ ID NOs: 20, 22, 24.

21. An isolated bispecific antigen-binding molecule, comprising:
(a) a first antigen-binding domain that specifically binds human 4-1BB wherein the first antigen-binding domain comprises HCDR1, HCDR2, HCDR3 domains, respectively, comprising the amino acid sequences of SEQ ID NOs: 12, 14, 16, and LCDR1, LCDR2, LCDR3 domains, respectively, comprising the amino acid sequences of SEQ ID NOs: 20, 22, 24; and
(b) a second antigen-binding domain that specifically binds human PD-L1 wherein the second antigen-binding domain comprises HCDR1, HCDR2, HCDR3 domains, respectively, comprising the amino acid sequences of SEQ ID NOs: 4, 6, 8, and LCDR1, LCDR2, LCDR3 domains, respectively, comprising the amino acid sequences of SEQ ID NOs: 20, 22, 24.

22. The isolated bispecific antigen-binding molecule of claim 18, comprising:
(a) a first antigen-binding domain that comprises a HCVR comprising the amino acid sequence of SEQ ID NO: 58, and a LCVR comprising the amino acid sequence of SEQ ID NO: 66; and
(b) a second antigen-binding domain that comprises a HCVR comprising the amino acid sequence of SEQ ID NO: 50, and a LCVR comprising the amino acid sequence of SEQ ID NO: 66.

23. The isolated bispecific antigen-binding molecule of claim 19, comprising:
(a) a first antigen-binding domain that comprises a HCVR comprising the amino acid sequence of SEQ ID NO: 42, and a LCVR comprising the amino acid sequence of SEQ ID NO: 18; and
(b) a second antigen-binding domain that comprises a HCVR comprising the amino acid sequence of SEQ ID NO: 2, and a LCVR comprising the amino acid sequence of SEQ ID NO: 18.

24. The isolated bispecific antigen-binding molecule of claim 20, comprising:
(a) a first antigen-binding domain that comprises a HCVR comprising the amino acid sequence of SEQ ID NO: 32, and a LCVR comprising the amino acid sequence of SEQ ID NO: 18; and
(b) a second antigen-binding domain that comprises a HCVR comprising the amino acid sequence of SEQ ID NO: 2, and a LCVR comprising the amino acid sequence of SEQ ID NO: 18.

25. The isolated bispecific antigen-binding molecule of claim 21, comprising:
(a) a first antigen-binding domain that comprises a HCVR comprising the amino acid sequence of SEQ ID NO: 10, and a LCVR comprising the amino acid sequence of SEQ ID NO: 18; and
(b) a second antigen-binding domain that comprises a HCVR comprising the amino acid sequence of SEQ ID NO: 2, and a LCVR comprising the amino acid sequence of SEQ ID NO: 18.

26. An isolated bispecific antigen-binding molecule that competes for binding to PD-L1 or binds to the same epitope on PD-L1 as a reference antibody, wherein the reference antibody comprises a first antigen-binding domain comprising an HCVR/LCVR pair comprising the amino acid sequences selected from the group consisting of SEQ ID NOs: 58/66, 42/18, 32/18 and 10/18 and a second antigen-binding domain comprising an HCVR/LCVR pair comprising the amino acid sequences selected from the group consisting of SEQ ID NOs: 50/66 and 2/18.

27. An isolated bispecific antigen-binding molecule that competes for binding to human 4-1BB or binds to the same epitope on human 4-1BB as a reference antibody, wherein the reference antibody comprises a first antigen-binding domain comprising an HCVR/LCVR pair comprising the amino acid sequences selected from the group consisting of SEQ ID NOs: 58/66, 42/18, 32/18 and 10/18 and a second antigen-binding domain comprising an HCVR/LCVR pair comprising the amino acid sequences selected from the group consisting of SEQ ID NOs: 50/66 and 2/18.

28. The isolated bispecific antigen-binding molecule of claim 1 that is a human bispecific antigen-binding molecule.

29. The isolated bispecific antigen-binding molecule of claim 1 that is a bispecific antibody.

30. The isolated bispecific antigen-binding molecule of 29, wherein the antibody comprises a human IgG heavy chain constant region attached, respectively, to the HCVR of each of the first antigen-binding domain and the second antigen-binding domain.

31. The isolated bispecific antigen-binding molecule of claim 30, wherein the heavy chain constant region is of isotype IgG1.

32. The isolated bispecific antigen-binding molecule of claim 30, wherein the heavy chain constant region is of isotype IgG4.

33. The isolated bispecific antigen-binding molecule of claim 30, wherein the heavy chain constant region attached to the HCVR of the first antigen-binding domain, or the heavy chain constant region attached to the HCVR of the second antigen-binding domain, but not both, contains an amino acid modification that reduces Protein A binding relative to a heavy chain of the same isotype without the modification.

34. The isolated bispecific antigen-binding molecule of claim 33, wherein the modification comprises a H435R substitution (EU numbering) in a heavy chain of isotype IgG1 or IgG4.

35. The isolated bispecific antigen-binding molecule of claim 33, wherein the modification comprises a H435R substitution and a Y436F substitution (EU numbering) in a heavy chain of isotype IgG1 or IgG4.

36. The isolated bispecific antigen-binding molecule of claim 29, wherein the bispecific antibody comprises a chimeric hinge that reduces Fcγ receptor binding relative to a wild-type hinge of the same isotype.

37. The isolated bispecific antigen-binding molecule of claim 29, wherein the antibody comprises a first heavy chain containing the HCVR of the first antigen-binding domain and a second heavy chain containing the HCVR of the second antigen-binding domain, wherein the first heavy chain comprises the amino acid sequence selected from the group consisting of SEQ ID NOs: 76, 48, 40 and 28; and the second heavy chain comprises the amino acid sequence selected from the group consisting of SEQ ID NOs: 74 and 26.

38. The isolated bispecific antigen-binding molecule of claim 37, wherein the antibody comprises a common light chain containing the LCVR of the first and second antigen-binding domains, wherein the common light chain comprises the amino acid sequence selected from the group consisting of SEQ ID NOs: 78 and 30.

39. The isolated bispecific antigen-binding molecule of claim 27, wherein the antibody comprises a first heavy chain containing the HCVR of the first antigen-binding domain and a second heavy chain containing the HCVR of the second antigen-binding domain, wherein the first heavy chain comprises the amino acid sequence of SEQ ID NO: 76 and the second heavy chain comprises the amino acid sequence of SEQ ID NO: 74.

40. The isolated bispecific antigen-binding molecule of claim 39, wherein the antibody comprises a common light chain containing the LCVR of the first and second antigen-binding domains, wherein the common light chain comprises the amino acid sequence of SEQ ID NOs: 78.

41. A bispecific antibody comprising a first antigen-binding domain that binds specifically to human 4-1BB and a second antigen-binding domain that binds specifically to human PD-L1, wherein the bispecific antibody comprises a first heavy chain comprising the amino acid sequence of SEQ ID NO: 76 paired with a common light chain comprising the amino acid sequence of SEQ ID NO: 78, and a second heavy chain comprising the amino acid sequence of SEQ ID NO: 74 paired with a common light chain comprising the amino acid sequence of SEQ ID NO: 78.

42. A bispecific antibody comprising a first antigen-binding domain that binds specifically to human 4-1BB and a second antigen-binding domain that binds specifically to human PD-L1, wherein the bispecific antibody comprises a first heavy chain comprising the amino acid sequence of SEQ ID NO: 48 paired with a common light chain comprising the amino acid sequence of SEQ ID NO: 30, and a second heavy chain comprising the amino acid sequence of SEQ ID NO: 26 paired with a common light chain comprising the amino acid sequence of SEQ ID NO: 30.

43. A bispecific antibody comprising a first antigen-binding domain that binds specifically to human 4-1BB and a second antigen-binding domain that binds specifically to human PD-L1, wherein the bispecific antibody comprises a first heavy chain comprising the amino acid sequence of SEQ ID NO: 40 paired with a common light chain comprising the amino acid sequence of SEQ ID NO: 30, and a second heavy chain comprising the amino acid sequence of SEQ ID NO: 26 paired with a common light chain comprising the amino acid sequence of SEQ ID NO: 30.

44. A bispecific antibody comprising a first antigen-binding domain that binds specifically to human 4-1BB and a second antigen-binding domain that binds specifically to human PD-L1, wherein the bispecific antibody comprises a first heavy chain comprising the amino acid sequence of SEQ ID NO: 28 paired with a common light chain comprising the amino acid sequence of SEQ ID NO: 30, and a second heavy chain comprising the amino acid sequence of SEQ ID NO: 26 paired with a common light chain comprising the amino acid sequence of SEQ ID NO: 30.

45. The bispecific antibody of claim 41 that is a human antibody.

46. A pharmaceutical composition comprising the bispecific antigen-binding molecule of claim 1, and a pharmaceutically acceptable carrier or diluent.

47. A pharmaceutical composition comprising the bispecific antibody of claim 41, and a pharmaceutically acceptable carrier or diluent.

48. A method for making the bispecific antigen-binding molecule of claim 1 comprising:
(a) introducing one or more nucleic acid molecules comprising nucleic acid sequences encoding the immunoglobulin chains of said bispecific antigen-binding molecule into a host cell;
(b) culturing the host cell under conditions favorable to expression of the nucleic acid molecules; and
(c) optionally, isolating the bispecific antigen-binding molecule or immunoglobulin chain from the host cell and/or medium in which the host cell is grown.

49. The method of claim 48, wherein the host cell is a Chinese hamster ovary (CHO) cell.

50. The method of claim 48, further comprising formulating the antigen-binding molecule as a pharmaceutical composition comprising an acceptable carrier.

51. (canceled)

52. A nucleic acid molecule comprising a nucleotide sequence encoding the bispecific antigen-binding molecule of claim 1, or a set of nucleic acid molecules comprising nucleotide sequences encoding the HCVR of the first antigen-binding domain that specifically binds to human 4-1BB, the HCVR of the second antigen-binding domain that specifically binds to human PD-L1, and the LCVR of claim 1.

53. An expression vector comprising the nucleic acid molecule of claim 52, or a set of expression vectors comprising the set of nucleic acid molecules of claim 52.

54. A host cell comprising the expression vector or set of expression vectors of claim 53.

55. The host cell of claim 54, wherein the host cell is a Chinese hamster ovary (CHO) cell.

56. A method of producing a bispecific antigen-binding molecule that binds to PD-L1 and 4-1BB comprising:
(a) culturing the host cell of claim 54 under conditions favorable for production of the bispecific antigen-binding molecule; and
(b) optionally, isolating the antigen-binding molecule or immunoglobulin chain from the host cell and/or medium in which the host cell is grown

57. The method of claim 56, wherein the host cell is a CHO cell.

58. The method of claim 56, further comprising formulating the antigen-binding molecule as a pharmaceutical composition comprising an acceptable carrier.

59-67. (canceled)

68. A method of inhibiting growth of a tumor in a subject, comprising administering an isolated bispecific antigen-binding molecule of claim 1 to the subject.

69. The method of claim 68, wherein the tumor is esophageal carcinoma, lung squamous cell carcinoma, lung adenocarcinoma, cervical squamous cell carcinoma, endometrial adenocarcinoma, bladder urothelial carcinoma, lung cancer, non-small cell lung cancer, colorectal cancer, rectal cancer, endometrial cancer, skin cancer, head & neck squamous cell carcinoma, brain cancer, glioblastoma multiforme, breast cancer, gastroesophageal cancer, gastroesophageal adenocarcinoma, hepatocellular carcinoma, prostate cancer, ovarian cancer, a B cell cancer, a T cell cancer, leukemia, pancreatic cancer, colon cancer, melanoma, basal cell carcinoma, cervical cancer, diffuse large B cell lymphoma, or multiple myeloma.

70. The method of claim 68, wherein the tumor expresses PD-L1.

71. The method of claim 68, further comprising administering a second therapeutic agent or therapeutic regimen.

72. The method of claim 71, wherein the second therapeutic agent or therapeutic regimen comprises a chemotherapeutic drug, a DNA alkylator, an immunomodulator, a proteasome inhibitor, a histone deacetylase inhibitor, radiotherapy, surgery, a stem cell transplant, a bispecific antibody that interacts with a tumor associated antigen (TAA) and a T cell or immune cell antigen, an antibody drug conjugate, an oncolytic virus, a bispecific antibody conjugated to an anti-tumor agent, a VEGF inhibitor, a checkpoint inhibitor, a GITR agonist, a CD27 agonist, a 4-1BB activator, a PD-1 inhibitor, a CTLA-4 inhibitor, an EGFR inhibitor, Ang2 inhibitor, a MUC16 inhibitor, a cancer vaccine, a cytokine, a modified IL2, a modified IL12, IL4 inhibitor, IL6 inhibitor, a corticosteroid, or combinations thereof.

73. The method of claim 72, wherein the T cell or immune cell antigen is CD3.

74. The method of claim 72, wherein the TAA is selected from the group consisting of AFP, ALK, BAGE proteins, BCMA, BIRC5 (survivin), BIRC7, β-catenin, brc-abl, BRCA1, BORIS, CA9, carbonic anhydrase IX, caspase-8, CALR, CCR5, CD19, CD20 (MS4A1), CD22, CD30, CD40, CDK4, CEA, CTLA4, cyclin-B1, CYP1B1, EGFR, EGFRVIII, ErbB2/Her2, ErbB3, ErbB4, ETV6-AML, EpCAM, EphA2, Fra-1, FOLR1, GAGE proteins, GD2, GD3, GloboH, glypican-3, GM3, gp100, Her2, HLA/B-raf, HLA/k-ras, HLA/MAGE-A3, hTERT, LMP2, MAGE proteins, MART-1, mesothelin, ML-IAP, Muc1, Muc2, Muc3, Muc4, Muc5, Muc16 (CA-125), MUM1, NA17, NY-BR1, NY-BR62, NY-BR85, NY-ESO1, OX40, p15, p53, PAP, PAX3, PAX5, PCTA-1, PLAC1, PRLR, PRAME, PSMA (FOLH1), RAGE proteins, Ras, RGS5, Rho, SART-1, SART-3, STEAP1, STEAP2, TAG-72, TGF-β, TMPRSS2, Thompson-nouvelle antigen (Tn), TRP-1, TRP-2, tyrosinase, and uroplakin-3.

75-78. (canceled)
